(19) United States
(12) Patent Application Publication
(10) Pub. No.: US 2024/0368612 A1
(43) Pub. Date: Nov. 7, 2024

(54) PLANT REGULATORY ELEMENTS AND METHODS OF USE THEREOF

(71) Applicant: PIONEER HI-BRED INTERNATIONAL, INC., JOHNSTON, IA (US)

(72) Inventors: ANDREW CROW, GRIMES, IA (US); SCOTT DIEHN, WEST DES MOINES, IA (US); LYNNE SIMS, POLK CITY, IA (US)

(73) Assignee: PIONEER HI-BRED INTERNATIONAL, INC., JOHNSTON, IA (US)

(21) Appl. No.: 18/780,871

(22) Filed: Jul. 23, 2024

Related U.S. Application Data
(63) Continuation-in-part of application No. 16/311,893, filed on Dec. 20, 2018, now Pat. No. 12,065,653.
(60) Provisional application No. 62/354,374, filed on Jun. 24, 2016.

(51) Int. Cl. C12N 15/82

(52) U.S. Cl. CPC C12N 15/8227; C12N 15/8271

(57) ABSTRACT

The present disclosure relates to the field of plant molecular biology, more particularly to regulation of gene expression in plants.

### CROSS-REFERENCE TO RELATED APPLICATIONS

This application claims priority to U.S. patent application Ser. No. 16/311,893, filed on Dec. 20, 2018, which claims priority to International Patent Application PCT/US2017/036503 filed on Jun. 8, 2017, which claims priority to U.S. Provisional Application No. 62/354,374, filed Jun. 24, 2016, the contents of which are herein incorporated by reference in their entirety.

### REFERENCE TO SEQUENCE LISTING SUBMITTED ELECTRONICALLY

A sequence listing having the file name “104659-US-CON-1.XML” created on Jul. 18, 2024, and having a size of 262,000 bytes is filed in XML format concurrently with the specification. The sequence listing is part of the specification as originally filed and is herein incorporated by reference in its entirety.

### FIELD

The present disclosure relates to the field of plant molecular biology, more particularly to the regulation of gene expression in plants.

### BACKGROUND

Expression of heterologous DNA sequences in a plant host is dependent upon the presence of operably linked regulatory elements that are functional within the plant host. Choice of promoter sequence may determine when and where within the organism a heterologous DNA sequence is expressed. Where expression in specific tissues or organs is desired, tissue-preferred promoters may be used. Where gene expression in response to a stimulus is desired, inducible promoters are the regulatory element of choice. In contrast, where continuous expression is desired throughout the cells of a plant, constitutive promoters are utilized. Additional regulatory sequences upstream and/or downstream from the core promoter sequence may be included in the expression constructs of transformation vectors to bring about varying levels of expression of heterologous nucleotide sequences in a transgenic plant.

Frequently it is desirable to express a DNA sequence in particular tissues or organs of a plant. For example, increased resistance of a plant to infection by soil-and air-borne pathogens might be accomplished by genetic manipulation of the plant's genome to comprise a tissue-preferred promoter operably linked to a heterologous pathogen-resistance gene such that pathogen-resistance proteins are produced in the desired plant tissue. Alternatively, it might be desirable to inhibit expression of a native DNA sequence within a plant's tissues to achieve a desired phenotype. In this case, such inhibition might be accomplished with transformation of the plant to comprise a tissue-preferred promoter operably linked to an antisense nucleotide sequence, such that expression of the antisense sequence produces an RNA transcript that interferes with translation of the mRNA of the native DNA sequence.

Genetically altering plants through the use of genetic engineering techniques and thus producing a plant with useful traits may require the availability of a variety of regulatory elements. An accumulation of promoters and other regulatory elements would enable the investigator to express at desired levels and cellular locales recombinant DNA molecules. Therefore, a collection of promoters would allow for a new trait to be expressed at the desired level in the desired tissue. Thus, isolation, characterization, and creation of hybrid regulatory elements that may produce an expression pattern that is unique and serve as regulatory regions for expression of heterologous nucleotide sequences of interest are useful for the genetic manipulation of plants.

### BRIEF SUMMARY

Compositions and methods for regulating expression of a heterologous polynucleotide sequence of interest in a plant or plant cell are provided. Synthetic DNA molecules comprising novel polynucleotide sequences for regulatory elements having gene regulatory activity are provided. In some embodiments the regulatory element has promoter activity initiating transcription in the plant cell. Certain embodiments comprise the nucleotide sequences set forth in SEQ ID NOs: 1-74. Also included are functional fragments, segments, or variants of the sequences set forth in SEQ ID NOs: 1-74 wherein said sequences initiate transcription in a plant cell, or a polynucleotide sequence comprising a sequence having at least 85% sequence identity to the sequences set forth in SEQ ID NOs: 1-74, wherein said sequences initiate transcription in the plant cell. Embodiments also include DNA constructs comprising a regulatory element operably linked to a heterologous nucleotide sequence of interest, wherein said regulatory element is capable of driving expression of said heterologous nucleotide sequence in a plant cell and said regulatory element comprises a nucleotide sequence disclosed herein. Embodiments further provide expression vectors, and plants or plant cells having stably incorporated into their genomes a DNA construct as is described above. Additionally, compositions include transgenic seed of such plants.

In one embodiment, hybrid regulatory elements are provided that produce an expression pattern in plants that is unique for each of the hybrid regulatory elements relative to any source or parent regulatory material from which the hybrid regulatory element is produced, wherein the hybrid regulatory element contains a segment of each parent regulatory element. In one embodiment, the hybrid regulatory element produces a tissue specific expression pattern that is different relative to the parent regulatory elements. In another embodiment, the hybrid regulatory elements provide a broadened expression pattern in plant tissues relative to narrower expression patterns expressed from a given set of parent regulatory elements. In another embodiment, hybrid regulatory elements may produce a constitutive expression pattern that differs from a non-constitutive expression pattern of the parent regulatory elements.

Methods and compositions are provided that relate to the construction of hybrid regulatory elements. In one embodiment, a hybrid regulatory element may be created using one or more previously characterized regulatory elements and assembling segments of the previously characterized one or more regulatory elements. In a further embodiment, the characterized regulatory elements may be evaluated for potential motifs to aid in defining segments that may be useful in a hybrid regulatory element. In one embodiment, the 3′ end of the hybrid promoter contains a TATA box from a parental regulatory element. In another embodiment, a method provided relates to expressing a nucleotide sequence in a plant or plant cell comprising introducing into the plant or the plant cell an expression cassette comprising a hybrid regulatory element operably linked to a nucleotide sequence of interest, wherein said hybrid regulatory element produces a tissue expression pattern of the nucleotide sequence of interest that is unique relative to each of the individual parental regulatory element tissue expression patterns.

In one embodiment, parent regulatory elements that may be used to make a hybrid regulatory element comprise SEQ ID NOs: 1-10, 31-34, and 61-74 or variants thereof. Also included are functional fragments, segments, or variants of the polynucleotide sequences set forth in SEQ ID NOs: 1-10, 31-34, and 61-74 wherein said polynucleotide sequences initiate transcription in a plant cell, or a polynucleotide sequence comprising a sequence having at least 85% sequence identity to the polynucleotide sequences set forth in SEQ ID NOs: 1-10, 31-34, and 61-74, and wherein said polynucleotide sequences initiate transcription in a plant cell. In another embodiment, parent regulatory elements that may be used to make a hybrid regulatory element comprise one or more of SEQ ID NOs: 1-74 or variants thereof.

Embodiments also include DNA constructs comprising a hybrid regulatory element operably linked to a heterologous nucleotide sequence of interest, wherein said hybrid regulatory element is capable of driving expression of said heterologous nucleotide sequence in a plant cell and said promoter comprises one of SEQ ID NOs: 1-74, or a functional fragment thereof, as disclosed herein. Embodiments further provide expression vectors, and plants or plant cells having stably incorporated into their genomes a DNA construct as is described above. Additionally, compositions include transgenic seed of such plants.

Downstream from the transcriptional initiation region of the regulatory element will be a sequence of interest that will provide for modification of the phenotype of the plant. Such modification includes modulating the production of an endogenous product as to amount, relative distribution, or the like, or production of an exogenous expression product, to provide for a novel or modulated function or product in the plant. For example, a heterologous polynucleotide sequence that encodes a gene product that confers resistance or tolerance to herbicide, salt, cold, drought, pathogen, nematodes or insects is encompassed.

In a further embodiment, a method for modulating expression of a gene in a stably transformed plant is provided, comprising the steps of (a) transforming a plant cell with a DNA construct comprising a regulatory element disclosed herein, or a functional fragment thereof, operably linked to at least one heterologous polynucleotide sequence; (b) growing the plant cell under plant growing conditions and (c) regenerating a stably transformed plant from the plant cell wherein expression of the linked nucleotide sequence alters the phenotype of the plant. In another embodiment, the DNA construct further comprises a heterologous enhancer element.

Expression cassettes comprising the regulatory element sequences of SEQ ID NOs: 1-74 operably linked to a heterologous nucleotide sequence of interest are provided. Additionally, provided are transformed plant cells, plant tissues, seeds, and plants.

### DESCRIPTION OF THE DRAWINGS

FIG. 1 Shows a distribution of motifs used to assist defining the region of the regulatory element segment that would be incorporated into a hybrid regulatory element. The vertical lines indicate motif positions, the white arrow indicates the segment used in a hybrid regulatory element, and the black arrow indicates the full-length parent regulatory element. A. A representation of SI-TIP2-3B (MOD 1) regulatory element (SEQ ID NO: 10) and the 5′ region upstream of the TATAA box selected for use as a segment in a hybrid regulatory element based on clustering of motifs, and B. A representation of BD-GRP3 PRO (MOD 1) regulatory element (SEQ ID NO: 2) and the 3′ downstream region including the TATAA box selected for use as a segment in a hybrid regulatory element based on clustering of motifs.

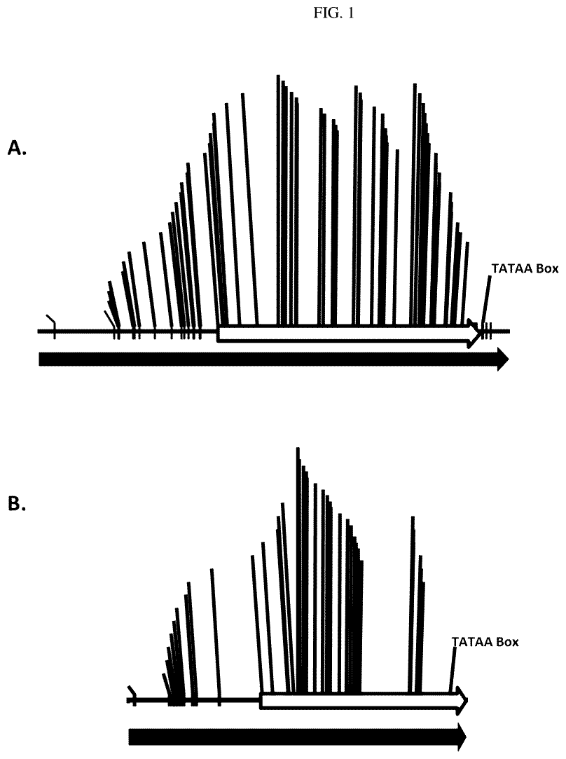

FIG. 2 Shows the segments used to make several root hybrid regulatory elements.

FIG. 3 Shows the segments used to make each aerial hybrid regulatory element.

### DETAILED DESCRIPTION

The article “a” and “an” are used herein to refer to one or more than one (i.e., to at least one) of the grammatical object of the article. By way of example, “an element” means one or more element.

The disclosure relates to compositions and methods drawn to plant regulatory elements and methods of their use. The compositions and methods further relate to hybrid regulatory elements and methods of making and using hybrid regulatory elements.

Plant tissues are complex organs that can be divided into different regions; for example, in root tissue the tissue regions include the cap, meristem, elongation region and mature region. These regions reflect different stages of cell identity and maturity. Genes in these cells are consequently activated and inactivated during development, and therefore may not be expressed simultaneously in all regions of the root or other tissue. However, combining pieces of regulatory elements from these genes as taught herein, a synthetic regulatory element can be created that drives expression in the different regions of a particular tissue. The regulatory elements provided may retain tissue specificity, although unique expression patterns may be created.

The disclosure relates to compositions and methods drawn to plant hybrid regulatory elements and methods of their use. The compositions comprise polynucleotide sequences for the regulatory elements set forth in SEQ ID NOs: 1-74, and fragments, variants and complements thereof. In one embodiment, the compositions comprise polynucleotide sequences for hybrid regulatory elements set forth in SEQ ID NOs: 11-30, 35-47, and 50-59, and fragments, variants and complements thereof.

Compositions may include the nucleotide sequences for the regulatory elements, fragments and variants thereof. In specific embodiments, the hybrid regulatory element sequences disclosed herein are useful for changing the expression pattern relative to a native regulatory element. The nucleotide sequences also find use in the construction of expression vectors for subsequent expression of a heterologous nucleotide sequence in a plant of interest or as probes for the isolation of other regulatory elements. One embodiment is provided for DNA constructs comprising a regulatory element polynucleotide sequence set forth in SEQ ID NOs: 1-10, 31-34, and 61-74, or a hybrid regulatory elements set forth in SEQ ID NOs: 11-30 and 35-60, or a functional fragment or variants thereof, operably linked to a heterologous polynucleotide sequence of interest, and any combinations thereof.

The term “regulatory element” refers to a nucleic acid molecule having gene regulatory activity, i.e. one that has the ability to affect the transcriptional and/or translational expression pattern of an operably linked transcribable polynucleotide. The term “gene regulatory activity” thus refers to the ability to affect the expression of an operably linked transcribable polynucleotide molecule by affecting the transcription and/or translation of that operably linked transcribable polynucleotide molecule. Gene regulatory activity may be positive and/or negative and the effect may be characterized by its temporal, spatial, developmental, tissue, environmental, physiological, pathological, cell cycle, and/or chemically responsive qualities as well as by quantitative or qualitative indications.

Regulatory elements such as promoters, enhancers, leaders, and intron regions are nucleic acid molecules that have gene regulatory activity and play an integral part in the overall expression of genes in living cells. Isolated regulatory elements, such as promoters and leaders that function in plants are therefore useful for modifying plant phenotypes through the methods of genetic engineering. A promoter is useful as a regulatory element for modulating the expression of an operably linked transcribable polynucleotide molecule.

As used herein, a “gene expression pattern” is any pattern of transcription of an operably linked nucleic acid molecule into a transcribed RNA molecule. Expression may be characterized by its temporal, spatial, developmental, tissue, environmental, physiological, pathological, cell cycle, and/or chemically responsive qualities as well as by quantitative or qualitative indications. The transcribed RNA molecule may be translated to produce a protein molecule or may provide an antisense or other regulatory RNA molecule, such as a dsRNA, a tRNA, an rRNA, a miRNA, and the like. As used herein, a “unique gene expression pattern” is any pattern of transcription of an operably linked nucleic acid molecule into a transcribed RNA molecule that differs from another regulatory element by its specific tissue or sub-tissue regional transcriptional expression pattern, or cell type or cell state expression pattern and not merely a difference in the quantitative amount of transcription. A “unique gene expression pattern,” as used herein, does not include a constitutive expression pattern. In one embodiment, a unique gene expression pattern includes a reduction or elimination of transcription of a heterologous nucleic acid in a particular tissue. In another embodiment, a unique gene expression pattern includes an increase or production of transcription of a heterologous nucleic acid in a particular tissue. As used herein, a “regional transcriptional expression pattern” or “sub-regional transcriptional expression pattern” is intended to mean any expression pattern that is not ubiquitous, only has detectable expression in certain regions of a certain tissue(s), and any other expression outside of the region or regions should be at nominal baseline levels (“ectopic expression”). The term “aerial expression” refers to a gene expression pattern of a polynucleotide in one or more above ground tissues of a plant. In one embodiment, heterologous polynucleotide sequence of interest is expressed in a ubiquitous root expression pattern or a ubiquitous aerial expression pattern. In one embodiment, aerial expression does not include expression in the pollen of a plant.

The regulatory element sequences or variants or fragments thereof, when operably linked to a heterologous nucleotide sequence of interest may drive constitutive expression of the heterologous polynucleotide sequence in the tissue of the plant expressing this construct. The term “constitutive expression,” means that expression of the heterologous nucleotide sequence is found throughout the plant or in a majority of tissues of the plant.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a nucleic acid molecule that is involved in recognition and binding of RNA polymerase II and other proteins (trans-acting transcription factors) to initiate transcription. A promoter may be initially isolated from the 5′ flanking region of a genomic copy of a gene. Alternately, promoters may be synthetically produced or manipulated DNA molecules. Regulatory elements may comprise promoters and promoter activity.

In one embodiment, fragments are provided of a regulatory element disclosed herein. Regulatory element fragments may exhibit promoter activity, and may be useful alone or in combination with other regulatory elements and regulatory element fragments, such as in constructing hybrid regulatory elements. In specific embodiments, fragments of a regulatory element are provided comprising, or alternatively consisting of or consisting essentially of, at least about 50, 95, 150, 250, 500, or about 750 or more contiguous nucleotides of a polynucleotide molecule having promoter activity disclosed herein. Such fragments may exhibit at least about 85 percent, about 90 percent, about 95 percent, about 98 percent, or about 99 percent, or greater, identity with a reference sequence when optimally aligned to the reference sequence. As used herein, the term “regulatory element segment” is a fragment of a regulatory element characterized by an abundance of recognizable regulatory element motifs using at least those described by Higo, K et al. (1998) Nucleic Acids Research (incorporated by reference in its entirety), wherein the regulatory element segment produces a desired or unique expression pattern when combined with at least one other regulatory element segment. As used herein, a “regulatory element segment” is used interchangeably with a “segment,” a “defined segment,” and “parent segment.” In one embodiment, an abundance of recognizable regulatory element motifs includes at least 25 motifs, at least 30 motifs, at least 40 motifs, or at least 50 motifs. In specific embodiments, segments of a regulatory element are provided comprising at least about 50, 95, 150, 250, 500, 750, 1000, 1250, 1500, or about 1750 contiguous nucleotides of a polynucleotide molecule disclosed herein. In another embodiment, a segment comprises at least about 50, 95, 150, 250, 500, 750, 1000, 1250, 1500, or about 1750 contiguous nucleotides of a polynucleotide molecule, but less than the full length of the parent polynucleotide molecule. In one embodiment, a regulatory element segment comprises a fragment of a parent regulatory element, wherein the fragment comprises not more than about 2000, 1900, 1800, 1700, 1600, 1500, 1400, 1300, 1200, 1100, 1000, or 500 contiguous nucleotides of a parent regulatory element. In a further embodiment, a hybrid regulatory element comprises contiguous polynucleotide segments, wherein the continuous polynucleotide segments initiate transcription in a plant cell and produces a unique tissue expression pattern. As used herein, an “assembly” means two or more operably linked regulatory element segments. In one embodiment, an assembly of segments includes segments that are contiguous. In another embodiment, an assembly of segments includes one or more of the segments are not contiguous, but still produce an expression pattern.

A regulatory element or a regulatory element segment may also be analyzed for the presence of known promoter motifs, i.e. DNA sequence characteristics, such as a TATAA-box and other known transcription factor binding site motifs. Identification of such known motifs may be used by one of skill in the art to design hybrid regulatory elements having a desired or unique expression pattern when compared to the source or parent regulatory element. Nucleotide sequence motifs found in regulatory elements have been previously characterized and are available in the PLACE database (Higo, K et al. (1998) Nucleic Acids Research; dna.affrc.go.jp/htdocs/PLACE/, which can be accessed on the world-wide web using the “www” prefix; See also, PCT Application Number WO 2014/164399). In one embodiment, a motif comprises a sequence of at least six nucleotides. In some embodiments, a regulatory element segment comprises about 15, 20, 25, 30, 35, 40, 45, 50, 75, 100, 125, or 150 motifs per 1000 nucleotides. In some embodiments, a regulatory element comprises at least one motif for about every 5, 10, 15, 20, 25, 30, 35, 40, 45, or 50 nucleotides.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box. A regulatory element may naturally comprise one or more enhancer elements that affect the transcription of an operably linked polynucleotide sequence. An isolated enhancer element may also be fused to a heterologous promoter to produce a heterologous promoter cis-element, which confers an aspect of the overall modulation of gene expression. A regulatory element or regulatory element fragment disclosed herein may comprise one or more enhancer elements that effect the transcription of operably linked genes. Many enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements may be identified by a number of techniques, including deletion analysis, i.e., deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain may be further studied by mutagenesis (or substitution) of one or more nucleotides or by other conventional methods. Enhancer elements may be obtained by chemical synthesis or by isolation from regulatory elements that include such elements, and they may be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed.

As used herein, the term “5′ flanking region” refers to a DNA molecule isolated from a genomic copy of a gene and is defined generally as a polynucleotide segment beginning at the protein coding sequence start site and extending 5′ through the 5′ untranslated region and into the promoter region. These sequences, or leaders, may be synthetically produced or manipulated DNA elements. A leader may be used as a 5′ regulatory element for modulating expression of an operably linked transcribable polynucleotide molecule. Leader molecules may be used with heterologous elements or with their native elements.

As used herein, the term “hybrid” refers to a single synthetic DNA molecule produced by fusing a first DNA molecule to a second DNA molecule, where neither first nor second DNA molecule would normally be found in that configuration, i.e. fused to the other. The hybrid DNA molecule is thus a new DNA molecule not normally found in nature. As used herein, the term “hybrid regulatory element” refers to a regulatory element produced through such manipulation of DNA molecules. A hybrid regulatory element may combine two or more DNA segments. Thus, the design, construction, and use of hybrid regulatory element according to the methods disclosed herein for modulating the expression of operably linked transcribable polynucleotide molecules are encompassed. In one embodiment, a hybrid regulatory element comprises two or more DNA segments. In another embodiment, a hybrid regulatory element comprises three or more DNA segments. In another embodiment, a hybrid regulatory element comprises four or more DNA segments. In one embodiment, a DNA fragment segment may be a parent segment. As used herein, a “segment,” and “parent segment” are interchangeable and intended to refer to fragments of native “parent regulatory elements” that have been analyzed for motifs that are predicted to produce a regional tissue expression pattern. A combination of parent segments or variants thereof, may result in a hybrid regulatory element expressing a gene of interest in a ubiquitous tissue expression pattern that is unique from each individual expression pattern of the parent regulatory elements. In one embodiment, a parent segment may be a variant of a parent regulatory element. In one embodiment, parent regulatory elements set forth in SEQ ID NOs: 1-10, 31-34, and 61-74 may be used as parent regulatory elements to generate parent segments and variants thereof. Also, included as parent regulatory elements are functional fragments, segments, or variants of the polynucleotide sequences set forth in SEQ ID NOs: 1-10, 31-34, and 61-74 wherein said polynucleotide sequences initiate transcription in a plant cell, and a polynucleotide sequence comprising a sequence having at least 85% sequence identity to the polynucleotide sequences set forth in SEQ ID NOs: 1-10, 31-34, and 61-74, wherein said polynucleotide sequences initiate transcription in a plant cell. In certain embodiments, a parent regulatory element may include a parent regulatory element originating from a plant. In another embodiment, a parent regulatory element may include a parent regulatory element originating from a bacteria or a virus.

Hybrid regulatory elements are provided that produce an expression pattern in plants that is unique relative to parent regulatory elements, wherein the hybrid regulatory element contains segments or fragments of more than one parent regulatory element. In one embodiment, the hybrid regulatory element produces a tissue specific expression pattern that is different relative to the parent regulatory elements. In another embodiment, the hybrid regulatory elements broaden the expression pattern to a ubiquitous expression pattern in a plant tissue relative to regional tissue expression patterns expressed from a given set of parent regulatory elements. In another embodiment, the hybrid regulatory elements express a narrower range of expression relative to a broader range of expression patterns expressed from a given set of parent regulatory elements. In another embodiment, the hybrid root regulatory elements may produce a constitutive expression pattern that differs from a non-constitutive expression pattern of the parent regulatory elements.

In one embodiment, the polynucleotide sequences disclosed herein, located within introns, or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. A post-transcriptional regulatory element may include elements that are active following transcription initiation, for example translational and transcriptional enhancers, translational and transcriptional repressors, and mRNA stability determinants.

The regulatory elements, or variants or fragments thereof, may be operatively associated with one or more heterologous regulatory elements in order to modulate the activity of the heterologous regulatory element. Such modulation includes enhancing or repressing transcriptional activity of the heterologous regulatory element, modulating post-transcriptional events, or either enhancing or repressing transcriptional activity of the heterologous regulatory element and modulating post-transcriptional events. For example, one or more regulatory elements, or fragments thereof, may be operatively associated with constitutive, inducible, or tissue specific promoters or fragment thereof, to modulate the activity of such promoters within desired tissues in plant cells.

The compositions may encompass isolated or recombinant nucleic acid. An “isolated” or “recombinant” nucleic acid molecule is used herein to refer to a nucleic acid sequence that is no longer in its natural environment, for example in an in vitro or in a heterologous recombinant bacterial or plant host cell. An isolated or recombinant nucleic acid molecule, or biologically active portion thereof, is substantially free of other cellular material or culture medium when produced by recombinant techniques, or substantially free of chemical precursors or other chemicals when chemically synthesized. An isolated or recombinant nucleic acid is free of sequences (optimally protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule may contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. The hybrid regulatory element sequences disclosed herein may be isolated from the 5′ untranslated region flanking their respective transcription initiation sites. As used herein, the terms “polynucleotide” and “nucleotide” are both intended to mean one or more nucleotide and may be used interchangeably in the singular or plural.

Fragments and variants of the disclosed regulatory element polynucleotide sequences are also encompassed by the present disclosure. As used herein, the term “fragment” refers to a portion of the nucleic acid sequence. Fragments of regulatory sequences may retain the biological activity of initiating transcription, more particularly driving transcription in a tissue specific or sub-tissue specific manner. Alternatively, fragments of a polynucleotide sequence that are useful as hybridization probes may not necessarily retain biological activity. Fragments of a polynucleotide sequence for the regulatory region may range from at least about 20 nucleotides, about 50 nucleotides, about 100 nucleotides, and up to one nucleotide less than the full length of any one of SEQ ID NOs: 1-74.

A biologically active portion of a regulatory element may be prepared by isolating a portion of the regulatory sequence, and assessing the promoter activity of the portion. Nucleic acid molecules that are fragments of a regulatory polynucleotide sequence comprise at least about 16, 50, 75, 100, 150, 200, 250, 300, 350, 400, 450, 500, 550, 600, 650, 700 or 800 nucleotides or up to the number of nucleotides present in a full-length regulatory sequence disclosed herein.

For polynucleotide sequences, a variant comprises a deletion and/or addition of one or more nucleotides at one or more internal sites within the native polynucleotide sequence and/or a substitution of one or more nucleotides at one or more sites in the native polynucleotide. For polynucleotide sequences, variants may be identified with the use of well-known molecular biology techniques, as, for example, with polymerase chain reaction (PCR) and hybridization techniques as outlined below. Variant polynucleotide sequences may include synthetically derived polynucleotide sequences, such as those generated, for example, by using site-directed mutagenesis. Generally, variants of a particular nucleotide sequence, including SEQ ID NOs: 1-74, of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular nucleotide sequence as determined by sequence alignment programs and parameters described elsewhere herein. A biologically active variant of a polynucleotide sequence of the disclosure may differ from that sequence by as few as 1-15 nucleic acid residues, as few as 1-10, as few as 6-10, as few as 5, as few as 4, 3, 2, or even 1 nucleic acid residue.

Variant polynucleotide sequences also encompass sequences derived from a mutagenic and recombinogenic procedure such as DNA shuffling. With such a procedure, regulatory element polynucleotide sequences may be manipulated to create new regulatory elements. In this manner, libraries of recombinant polynucleotides are generated from a population of related sequence polynucleotides comprising sequence regions that have substantial sequence identity and may be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotide sequences of the disclosure may be used to isolate corresponding sequences from other organisms, particularly other plants, more particularly other monocots. In this manner, methods such as PCR, hybridization and the like may be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers may be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers and the like.

In hybridization techniques, all or part of a known polynucleotide sequence is used as a probe that selectively hybridizes to other corresponding polynucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as 32P or any other detectable marker. Thus, for example, probes for hybridization may be made by labeling synthetic oligonucleotides based on the regulatory element sequences of the disclosure. Methods for preparation of probes for hybridization and for construction of genomic libraries are generally known in the art and are disclosed in Sambrook, supra.

For example, an entire regulatory element sequence disclosed herein, or one or more portions thereof, may be used as a probe capable of specifically hybridizing to corresponding regulatory element sequences and messenger RNAs. To achieve specific hybridization under a variety of conditions, such probes include sequences that are unique among regulatory element sequences and are generally at least about 10 nucleotides in length or at least about 20 nucleotides in length. Such probes may be used to amplify corresponding regulatory element sequences from a chosen plant by PCR. This technique may be used to isolate additional coding sequences from a desired organism or as a diagnostic assay to determine the presence of coding sequences in an organism. Hybridization techniques include hybridization screening of plated DNA libraries (either plaques or colonies, see, for example, Sambrook, supra).

In general, sequences that have promoter activity and hybridize to the polynucleotide sequences, and fragments thereof, disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 81%, 82%, 83%, 84%, 85%, 86%, 87%, 88%, 89%, 90%, 91%, 92%, 93%, 94%, 95%, 96%, 97%, 98%, 99% sequence identity.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Modifications of the isolated regulatory element sequences of the present disclosure may provide for a range of expression of the heterologous polynucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The regulatory elements disclosed herein may be used to increase or decrease expression, thereby resulting in a change in phenotype of the transformed plant. The polynucleotide sequences disclosed herein, as well as variants and fragments thereof, are useful in the genetic manipulation of any plant. The regulatory element sequences are useful in this aspect when operably linked with a heterologous nucleotide sequence whose expression is to be controlled to achieve a desired phenotypic response. The term “operably linked” means that the transcription or translation of the heterologous nucleotide sequence is under the influence of the regulatory element sequence. In this manner, the regulatory element sequences disclosed herein may be provided in expression cassettes along with heterologous polynucleotide sequences of interest for expression in the plant of interest, more particularly for expression in the reproductive tissue of the transformed plant.

The regulatory elements of the embodiments may be provided in DNA constructs for expression in the organism of interest. An “expression cassette” as used herein means a DNA construct comprising a regulatory element of the embodiments operably linked to a heterologous polynucleotide expressing a transcript or gene of interest. Such expression cassettes will comprise a transcriptional initiation region comprising one of the regulatory element polynucleotide sequences of the present disclosure, or variants or fragments thereof, operably linked to the heterologous nucleotide sequence. Such an expression cassette may be provided with a plurality of restriction sites for insertion of the polynucleotide sequence to be under the transcriptional regulation of the regulatory regions. The expression cassette may additionally contain selectable marker genes as well as 3′ termination regions.

The expression cassette may include, in the 5′-3′ direction of transcription, a transcriptional initiation region (i.e., a hybrid promoter, or variant or fragment thereof, of the disclosure), a translational initiation region, a heterologous polynucleotide sequence of interest, a translational termination region and optionally, a transcriptional termination region functional in the host organism. The regulatory regions (i.e., promoters, enhancers, transcriptional regulatory regions, and translational termination regions) and/or the polynucleotide of the embodiments may be native/analogous to the host cell or to each other. Alternatively, the regulatory regions and/or the polynucleotide of the embodiments may be heterologous to the host cell or to each other.

As used herein, “heterologous” in reference to a sequence is a sequence that originates from a foreign species or, if from the same species, is substantially modified from its native form in composition and/or genomic locus by deliberate human intervention. For example, a regulatory element operably linked to a heterologous polynucleotide is from a species different from the species from which the polynucleotide was derived or, if from the same/analogous species, one or both are substantially modified from their original form and/or genomic locus or the regulatory element is not the native regulatory element for the operably linked polynucleotide.

The termination region may be native with the transcriptional initiation region, may be native with the operably linked DNA sequence of interest, may be native with the plant host, or may be derived from another source (i.e., foreign or heterologous to the regulatory element, the DNA sequence being expressed, the plant host, or any combination thereof).

The regulatory elements disclosed herein, as well as variants and fragments thereof, are useful for genetic engineering of plants, e.g. for the production of a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of heterologous nucleic acid, including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a transgene of interest, the regeneration of a population of plants resulting from the insertion of the transgene into the genome of the plant and selection of a particular plant characterized by insertion into a particular genome location. An event is characterized phenotypically by the expression of the transgene. At the genetic level, an event is part of the genetic makeup of a plant. The term “event” also refers to progeny produced by a sexual cross between the transformant and another plant wherein the progeny include the heterologous DNA.

As used herein, the term plant includes whole plants, plant organs (e.g., leaves, stems, roots, etc.), plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the disclosure, provided that these parts comprise the introduced polynucleotides. As used herein, the term tis includes

The compositions and methods disclosed herein may be used for transformation of any plant species, including, but not limited to, monocots and dicots. Examples of plant species include corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed include, for example, pines such as loblolly pine (Pinus taeda), slash pine (Pinus elliotii), ponderosa pine (Pinusponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis). In specific embodiments, plants of may be crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Heterologous coding sequences expressed by a regulatory element sequence disclosed herein may be used for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results may be achieved by the expression of a heterologous polynucleotide sequence of interest comprising an appropriate gene product. In specific embodiments, the heterologous polynucleotide sequence of interest is an endogenous plant sequence whose expression level is increased in the plant or plant part. Results may be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant. In certain embodiments the expression patterns of the regulatory elements disclosed herein are useful for many types of screening.

General categories of polynucleotide sequences of interest that may be utilized with the regulatory sequences disclosed herein include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, environmental stress resistance (altered tolerance to cold, salt, drought, etc) and grain characteristics. Still other categories of transgenes include genes for inducing expression of exogenous products such as enzymes, cofactors, and hormones from plants and other eukaryotes as well as prokaryotic organisms. It is recognized that any gene of interest can be operably linked to the regulatory element of the disclosure and expressed in the plant.

By way of illustration, without intending to be limiting, the following is a list of other examples of the types of genes which may be used in connection with the regulatory elements disclosed herein.

1. Transgenes That Confer Resistance To Insects Or Disease And That Encode:

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example Jones, et al., (1994) Science 266:789 (cloning of the tomato Cf-9 gene for resistance to Cladosporium fulvum); Martin, et al., (1993) Science 262:1432 (tomato Pto gene for resistance to Pseudomonas syringae pv. tomato encodes a protein kinase); Mindrinos, et al., (1994) Cell 78:1089 (Arabidopsis RSP2 gene for resistance to Pseudomonas syringae); McDowell and Woffenden, (2003) Trends Biotechnol. 21(4):178-83 and Toyoda, et al., (2002) Transgenic Res. 11(6):567-82. A plant resistant to a disease is one that is more resistant to a pathogen as compared to the wild type plant.

(B) Genes encoding a Bacillus thuringiensis protein, a derivative thereof or a synthetic polypeptide modeled thereon. See, for example, Geiser, et al., (1986) Gene 48:109, who disclose the cloning and nucleotide sequence of a Bt delta-endotoxin gene. Moreover, DNA molecules encoding delta-endotoxin genes can be purchased from American Type Culture Collection (Rockville, Md.), for example, under ATCC® Accession Numbers 40098, 67136, 31995 and 31998. Other non-limiting examples of Bacillus thuringiensis transgenes being genetically engineered are given in the following patents and patent applications and hereby are incorporated by reference for this purpose: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Genes encoding pesticidal proteins may also be stacked including but are not limited to: insecticidal proteins from Pseudomonas sp. such as PSEEN3174 (Monalysin, (2011) PLoS Pathogens, 7:1-13), from Pseudomonas protegens strain CHA0 and Pf-5 (previously fluorescens) (Pechy-Tarr, (2008) Environmental Microbiology 10:2368-2386: GenBank Accession No. EU400157); from Pseudomonas Taiwanensis (Liu, et al., (2010) J. Agric. Food Chem. 58:12343-12349) and from Pseudomonas pseudoalcligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of U.S. Ser. No. 13/792,861; an AfIP-1A and/or AfIP-1B polypeptide of U.S. Ser. No. 13/800,233; a PHI-4 polypeptide of U.S. Ser. No. 13/839,702; a PIP-47 polypeptide of PCT Serial Number PCT/US14/51063; a PIP-72 polypeptide of PCT Serial Number PCT/US14/55128; a PtIP-50 polypeptide and a PtIP-65 polypeptide of PCT Publication Number WO2015/120270; a PtIP-83 polypeptide of PCT Publication Number WO2015/120276; a PtIP-96 polypeptide of PCT Serial Number PCT/US15/55502; and δ-endotoxins including, but not limited to, the Cry1, Cry2, Cry3, Cry4, Cry5, Cry6, Cry7, Cry8, Cry9, Cryl0, Cry11, Cry12, Cry13, Cry14, Cry15, Cry16, Cry17,

Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51 and Cry55 classes of δ-endotoxin genes and the B. thuringiensis cytolytic Cyt1 and Cyt2 genes. Members of these classes of B. thuringiensis insecticidal proteins include, but are not limited to Cry1Aa1 (Accession #AAA22353); Cry1Aa2 (Accession #Accession #AAA22552); Cry1Aa3 (Accession #BAA00257); Cry1Aa4 (Accession #CAA31886); Cry1Aa5 (Accession #BAA04468); Cry1Aa6 (Accession #AAA86265); Cry1Aa7 (Accession #AAD46139); Cry1Aa8 (Accession #126149); Cry1Aa9 (Accession #BAA77213); Cry1Aa10 (Accession #AAD55382); Cry1Aa11 (Accession #CAA70856); Cry1Aa12 (Accession #AAP80146); Cry1Aa13 (Accession #AAM44305); Cry1Aa14 (Accession #AAP40639); Cry1Aa15 (Accession #AAY66993); Cry1Aa16 (Accession #HQ439776); Cry1Aa17 (Accession #HQ439788); Cry1Aa18 (Accession #HQ439790); Cry1Aa19 (Accession #HQ685121); Cry1Aa20 (Accession #JF340156); Cry1Aa21 (Accession #JN651496); Cry1Aa22 (Accession #KC158223); Cry1Ab1 (Accession #AAA22330); Cry1Ab2 (Accession #AAA22613); Cry1Ab3 (Accession #AAA22561); Cry1Ab4 (Accession #BAA00071); Cry1Ab5 (Accession #CAA28405); Cry1Ab6 (Accession #AAA22420); Cry1Ab7 (Accession #CAA31620); Cry1Ab8 (Accession #AAA22551); Cry1Ab9 (Accession #CAA38701); Cry1Ab10 (Accession #A29125); Cry1Ab11 (Accession #I12419); Cry1Ab12 (Accession #AAC64003); Cry1Ab13 (Accession #AAN76494); Cry1Ab14 (Accession #AAG16877); Cry1Ab15 (Accession #AAO13302); Cry1Ab16 (Accession #AAK55546); Cry1Ab17 (Accession #AAT46415); Cry1Ab18 (Accession #AAQ88259); Cry1Ab19 (Accession #AAW31761); Cry1Ab20 (Accession #ABB72460); Cry1Ab21 (Accession #ABS18384); Cry1Ab22 (Accession #ABW87320); Cry1Ab23 (Accession #HQ439777); Cry1Ab24 (Accession #HQ439778); Cry1Ab25 (Accession #HQ685122); Cry1Ab26 (Accession #HQ847729); Cry1Ab27 (Accession #JN135249); Cry1Ab28 (Accession #JN135250); Cry1Ab29 (Accession #JN135251); Cry1Ab30 (Accession #JN135252); Cry1Ab31 (Accession #JN135253); Cry1Ab32 (Accession #JN135254); Cry1Ab33 (Accession #AAS93798); Cry1Ab34 (Accession #KC156668); Cry1Ab-like (Accession #AAK14336); Cry1Ab-like (Accession #AAK14337); Cry1Ab-like (Accession #AAK14338); Cry1Ab-like (Accession #ABG88858); Cry1Ac1 (Accession #AAA22331); Cry1Ac2 (Accession #AAA22338); Cry1Ac3 (Accession #CAA38098); Cry1Ac4 (Accession #AAA73077); Cry1Ac5 (Accession #AAA22339); Cry1Ac6 (Accession #AAA86266); Cry1Ac7 (Accession #AAB46989); Cry1Ac8 (Accession #AAC44841); Cry1Ac9 (Accession #AAB49768); Cry1Ac10 (Accession #CAA05505); Cry1Ac11 (Accession #CAA10270); Cry1Ac12 (Accession #112418); Cry1Ac13 (Accession #AAD38701); Cry1Ac14 (Accession #AAQ06607); Cry1Ac15 (Accession #AAN07788); Cry1Ac16 (Accession #AAU87037); Cry1Ac17 (Accession #AAX18704); Cry1Ac18 (Accession #AAY88347); Cry1Ac19 (Accession #ABD37053); Cry1Ac20 (Accession #ABB89046); Cry1Ac21 (Accession #AAY66992); Cry1Ac22 (Accession #ABZ01836); Cry1Ac23 (Accession #CAQ30431); Cry1Ac24 (Accession #ABL01535); Cry1Ac25 (Accession #FJ513324); Cry1Ac26 (Accession #FJ617446); Cry1Ac27 (Accession #FJ617447); Cry1Ac28 (Accession #ACM90319); Cry1Ac29 (Accession #DQ438941); Cry1Ac30 (Accession #GQ227507); Cry1Ac31 (Accession #GU446674); Cry1Ac32 (Accession #HM061081); Cry1Ac33 (Accession #GQ866913); Cry1Ac34 (Accession #HQ230364); Cry1Ac35 (Accession #JF340157); Cry1Ac36 (Accession #JN387137); Cry1Ac37 (Accession #JQ317685); Cry1Ad1 (Accession #AAA22340); Cry1Ad2 (Accession #CAA01880); Cry1Ae1 (Accession #AAA22410); Cry1Af1 (Accession #AAB82749); Cry1Ag1 (Accession #AAD46137); Cry1Ah1 (Accession #AAQ14326); Cry1Ah2 (Accession #ABB76664); Cry1Ah3 (Accession #HQ439779); Cry1Ai1 (Accession #AA039719); Cry1Ai2 (Accession #HQ439780); Cry1A-like (Accession #AAK14339); Cry1Ba1 (Accession #CAA29898); Cry1Ba2 (Accession #CAA65003); Cry1Ba3 (Accession #AAK63251); Cry1Ba4 (Accession #AAK51084); Cry1Ba5 (Accession #ABO20894); Cry1Ba6 (Accession #ABL60921); Cry1Ba7 (Accession #HQ439781); Cry1Bb1 (Accession #AAA22344); Cry1Bb2 (Accession #HQ439782); Cry1Bc1 (Accession #CAA86568); Cry1Bd1 (Accession #AAD10292); Cry1Bd2 (Accession #AAM93496); Cry1Be1 (Accession #AAC32850); Cry1Be2 (Accession #AAQ52387); Cry1Be3 (Accession #ACV96720); Cry1Be4 (Accession #HM070026); Cry1Bf1 (Accession #CAC50778); Cry1Bf2 (Accession #AAQ52380); Cry1Bg1 (Accession #AA039720); Cry1Bh1 (Accession #HQ589331); Cry1Bi1 (Accession #KC156700); Cry1Ca1 (Accession #CAA30396); Cry1Ca2 (Accession #CAA31951); Cry1Ca3 (Accession #AAA22343); Cry1Ca4 (Accession #CAA01886); Cry1Ca5 (Accession #CAA65457); Cry1Ca6 [1] (Accession #AAF37224); Cry1Ca7 (Accession #AAG50438); Cry1Ca8 (Accession #AAM00264); Cry1Ca9 (Accession #AAL79362); Cry1Ca10 (Accession #AAN16462); Cry1Ca11 (Accession #AAX53094); Cry1Ca12 (Accession #HM070027); Cry1Ca13 (Accession #HQ412621); Cry1Ca14 (Accession #JN651493); Cry1Cb1 (Accession #M97880); Cry1Cb2 (Accession #AAG35409); Cry1Cb3 (Accession #ACD50894); Cry1Cb-like (Accession #AAX63901); Cry1Da1 (Accession #CAA38099); Cry1Da2 (Accession #176415); Cry1Da3 (Accession #HQ439784); Cry1Db1 (Accession #CAA80234); Cry1Db2 (Accession #AAK48937); Cry1Dc1 (Accession #ABK35074); Cry1Ea1 (Accession #CAA37933); Cry1Ea2 (Accession #CAA39609); Cry1Ea3 (Accession #AAA22345); Cry1Ea4 (Accession #AAD04732); Cry1Ea5 (Accession #A15535); Cry1Ea6 (Accession #AAL50330); Cry1Ea7 (Accession #AAW72936); Cry1Ea8 (Accession #ABX11258); Cry1Ea9 (Accession #HQ439785); Cry1Ea10 (Accession #ADR00398); Cry1Ea11 (Accession #JQ652456); Cry1Eb1 (Accession #AAA22346); Cry1Fa1 (Accession #AAA22348); Cry1Fa2 (Accession #AAA22347); Cry1Fa3 (Accession #HM070028); Cry1Fa4 (Accession #HM439638); Cry1Fb1 (Accession #CAA80235); Cry1Fb2 (Accession #BAA25298); Cry1Fb3 (Accession #AAF21767); Cry1Fb4 (Accession #AAC10641); Cry1Fb5 (Accession #AAO13295); Cry1Fb6 (Accession #ACD50892); Cry1Fb7 (Accession #ACD50893); Cry1Ga1 (Accession #CAA80233); Cry1Ga2 (Accession #CAA70506); Cry1Gb1 (Accession #AAD10291); Cry1Gb2 (Accession #AAO13756); Cry1Gc1 (Accession #AAQ52381); Cry1Ha1 (Accession #CAA80236); Cry1Hb1 (Accession #AAA79694); Cry1Hb2 (Accession #HQ439786); Cry1H-like (Accession #AAF01213); Cryllal (Accession #CAA44633); Cry1Ia2 (Accession #AAA22354); Cry1Ia3 (Accession #AAC36999); Cry1Ia4 (Accession #AAB00958); Cry1Ia5 (Accession #CAA70124); Cry1Ia6 (Accession #AAC26910); Cry1Ia7 (Accession #AAM73516); Cry1Ia8 (Accession #AAK66742); Cry1Ia9 (Accession #AAQ08616); Cry1Ia10 (Accession #AAP86782); Cry1Ia11 (Accession #CAC85964); Cry1Ia12 (Accession #AAV53390); Cry1Ia13 (Accession #ABF83202); Cry1Ia14 (Accession #ACG63871); Cry1Ia15 (Accession #FJ617445); Cry1Ia16 (Accession #FJ617448); Cry1Ia17 (Accession #GU989199); Cry1Ia18 (Accession #ADK23801); Cry1Ia19 (Accession #HQ439787); Cry1Ia20 (Accession #JQ228426); Cry1Ia21 (Accession #JQ228424); Cry1Ia22 (Accession #JQ228427); Cry1Ia23 (Accession #JQ228428); Cry1Ia24 (Accession #JQ228429); Cry1Ia25 (Accession #JQ228430); Cry1Ia26 (Accession #JQ228431); Cry1Ia27 (Accession #JQ228432); Cry1Ia28 (Accession #JQ228433); Cry1Ia29 (Accession #JQ228434); Cry1Ia30 (Accession #JQ317686); Cry1Ia31 (Accession #JX944038); Cry1Ia32 (Accession #JX944039); Cry1Ia33 (Accession #JX944040); Cry1Ib1 (Accession #AAA82114); Cry1Ib2 (Accession #ABW88019); Cry1Ib3 (Accession #ACD75515); Cry1Ib4 (Accession #HM051227); Cry1Ib5 (Accession #HM070028); Cry1Ib6 (Accession #ADK38579); Cry1Ib7 (Accession #JN571740); Cry1Ib8 (Accession #JN675714); Cry1Ib9 (Accession #JN675715); Cry1Ib10 (Accession #JN675716); Cry1Ib11 (Accession #JQ228423); Cry1Icl (Accession #AAC62933); Cry1Ic2 (Accession #AAE71691); Cry1Id1 (Accession #AAD44366); Cry1Id2 (Accession #JQ228422); Cry1Ie1 (Accession #AAG43526); Cry1Ie2 (Accession #HM439636); Cry1Ie3 (Accession #KC156647); Cry1Ie4 (Accession #KC156681); Cry1Ifl (Accession #AAQ52382); Cry1Ig1 (Accession #KC156701); Cry1I-like (Accession #AAC31094); Cry1I-like (Accession #ABG88859); Cry1Ja1 (Accession #AAA22341); Cry1Ja2 (Accession #HM070030); Cry1Ja3 (Accession #JQ228425); Cry1Jb1 (Accession #AAA98959); Cry1Jc1 (Accession #AAC31092); Cry1Jc2 (Accession #AAQ52372); Cry1Jd1 (Accession #CAC50779); Cry1Ka1 (Accession #AAB00376); Cry1Ka2 (Accession #HQ439783); Cry1La1 (Accession #AAS60191); Cry1La2 (Accession #HM070031); Cry1Ma1 (Accession #FJ884067); Cry1Ma2 (Accession #KC156659); Cry1Na1 (Accession #KC156648); Cry1Nb1 (Accession #KC156678); Cry1-like (Accession #AAC31091); Cry2Aa1 (Accession #AAA22335); Cry2Aa2 (Accession #AAA83516); Cry2Aa3 (Accession #D86064); Cry2Aa4 (Accession #AAC04867); Cry2Aa5 (Accession #CAA10671); Cry2Aa6 (Accession #CAA10672); Cry2Aa7 (Accession #CAA10670); Cry2Aa8 (Accession #AAO13734); Cry2Aa9 (Accession #AAO13750); Cry2Aa10 (Accession #AAQ04263); Cry2Aa11 (Accession #AAQ52384); Cry2Aa12 (Accession #ABI83671); Cry2Aa13 (Accession #ABL01536); Cry2Aa14 (Accession #ACF04939); Cry2Aa15 (Accession #JN426947); Cry2Ab1 (Accession #AAA22342); Cry2Ab2 (Accession #CAA39075); Cry2Ab3 (Accession #AAG36762); Cry2Ab4 (Accession #AAO13296); Cry2Ab5 (Accession #AAQ04609); Cry2Ab6 (Accession #AAP59457); Cry2Ab7 (Accession #AAZ66347); Cry2Ab8 (Accession #ABC95996); Cry2Ab9 (Accession #ABC74968); Cry2Ab10 (Accession #EF157306); Cry2Ab11 (Accession #CAM84575); Cry2Ab12 (Accession #ABM21764); Cry2Ab13 (Accession #ACG76120); Cry2Ab14 (Accession #ACG76121); Cry2Ab15 (Accession #HM037126); Cry2Ab16 (Accession #GQ866914); Cry2Ab17 (Accession #HQ439789); Cry2Ab18 (Accession #JN135255); Cry2Ab19 (Accession #JN135256); Cry2Ab20 (Accession #JN135257); Cry2Ab21 (Accession #JN135258); Cry2Ab22 (Accession #JN135259); Cry2Ab23 (Accession #JN135260); Cry2Ab24 (Accession #JN135261); Cry2Ab25 (Accession #JN415485); Cry2Ab26 (Accession #JN426946); Cry2Ab27 (Accession #JN415764); Cry2Ab28 (Accession #JN651494); Cry2Ac1 (Accession #CAA40536); Cry2Ac2 (Accession #AAG35410); Cry2Ac3 (Accession #AAQ52385); Cry2Ac4 (Accession #ABC95997); Cry2Ac5 (Accession #ABC74969); Cry2Ac6 (Accession #ABC74793); Cry2Ac7 (Accession #CAL18690); Cry2Ac8 (Accession #CAM09325); Cry2Ac9 (Accession #CAM09326); Cry2Ac10 (Accession #ABN15104); Cry2Ac11 (Accession #CAM83895); Cry2Ac12 (Accession #CAM83896); Cry2Ad1 (Accession #AAF09583); Cry2Ad2 (Accession #ABC86927); Cry2Ad3 (Accession #CAK29504); Cry2Ad4 (Accession #CAM32331); Cry2Ad5 (Accession #CAO78739); Cry2Ae1 (Accession #AAQ52362); Cry2Af1 (Accession #ABO30519); Cry2Af2 (Accession #GQ866915); Cry2Ag1 (Accession #ACH91610); Cry2Ah1 (Accession #EU939453); Cry2Ah2 (Accession #ACL80665); Cry2Ah3 (Accession #GU073380); Cry2Ah4 (Accession #KC156702); Cry2Ai1 (Accession #FJ788388); Cry2Aj (Accession #); Cry2Ak1 (Accession #KC156660); Cry2Ba1 (Accession #KC156658); Cry3Aa1 (Accession #AAA22336); Cry3Aa2 (Accession #AAA22541); Cry3Aa3 (Accession #CAA68482); Cry3Aa4 (Accession #AAA22542); Cry3Aa5 (Accession #AAA50255); Cry3Aa6 (Accession #AAC43266); Cry3Aa7 (Accession #CAB41411); Cry3Aa8 (Accession #AAS79487); Cry3Aa9 (Accession #AAW05659); Cry3Aa10 (Accession #AAU29411); Cry3Aa11 (Accession #AAW82872); Cry3Aa12 (Accession #ABY49136); Cry3Ba1 (Accession #CAA34983); Cry3Ba2 (Accession #CAA00645); Cry3Ba3 (Accession #JQ397327); Cry3Bb1 (Accession #AAA22334); Cry3Bb2 (Accession #AAA74198); Cry3Bb3 (Accession #115475); Cry3Ca1 (Accession #CAA42469); Cry4Aa1 (Accession #CAA68485); Cry4Aa2 (Accession #BAA00179); Cry4Aa3 (Accession #CAD30148); Cry4Aa4 (Accession #AFB18317); Cry4A-like (Accession #AAY96321); Cry4Ba1 (Accession #CAA30312); Cry4Ba2 (Accession #CAA30114); Cry4Ba3 (Accession #AAA22337); Cry4Ba4 (Accession #BAA00178); Cry4Ba5 (Accession #CAD30095); Cry4Ba-like (Accession #ABC47686); Cry4Ca1 (Accession #EU646202); Cry4Cb1 (Accession #FJ403208); Cry4Cb2 (Accession #FJ597622); Cry4Cc1 (Accession #FJ403207); Cry5Aa1 (Accession #AAA67694); Cry5Ab1 (Accession #AAA67693); Cry5Ac1 (Accession #134543); Cry5Ad1 (Accession #ABQ82087); Cry5Ba1 (Accession #AAA68598); Cry5Ba2 (Accession #ABW88931); Cry5Ba3 (Accession #AFJ04417); Cry5Ca1 (Accession #HM461869); Cry5Ca2 (Accession #ZP_04123426); Cry5Da1 (Accession #HM461870); Cry5Da2 (Accession #ZP_04123980); Cry5Ea1 (Accession #HM485580); Cry5Ea2 (Accession #ZP_04124038); Cry6Aa1 (Accession #AAA22357); Cry6Aa2 (Accession #AAM46849); Cry6Aa3 (Accession #ABH03377); Cry6Ba1 (Accession #AAA22358); Cry7Aa1 (Accession #AAA22351); Cry7Ab1 (Accession #AAA21120); Cry7Ab2 (Accession #AAA21121); Cry7Ab3 (Accession #ABX24522); Cry7Ab4 (Accession #EU380678); Cry7Ab5 (Accession #ABX79555); Cry7Ab6 (Accession #ACI44005); Cry7Ab7 (Accession #ADB89216); Cry7Ab8 (Accession #GU145299); Cry7Ab9 (Accession #ADD92572); Cry7Ba1 (Accession #ABB70817); Cry7Bb1 (Accession #KC156653); Cry7Ca1 (Accession #ABR67863); Cry7Cb1 (Accession #KC156698); Cry7Da1 (Accession #ACQ99547); Cry7Da2 (Accession #HM572236); Cry7Da3 (Accession #KC156679); Cry7Ea1 (Accession #HM035086); Cry7Ea2 (Accession #HM132124); Cry7Ea3 (Accession #EEM19403); Cry7Fa1 (Accession #HM035088); Cry7Fa2 (Accession #EEM19090); Cry7Fb1 (Accession #HM572235); Cry7Fb2 (Accession #KC156682); Cry7Ga1 (Accession #HM572237); Cry7Ga2 (Accession #KC156669); Cry7Gb1 (Accession #KC156650); Cry7Gc1 (Accession #KC156654); Cry7Gd1 (Accession #KC156697); Cry7Ha1 (Accession #KC156651); Cry7Ia1 (Accession #KC156665); Cry7Ja1 (Accession #KC156671); Cry7Ka1 (Accession #KC156680); Cry7Kb1 (Accession #BAM99306); Cry7La1 (Accession #BAM99307); Cry8Aa1 (Accession #AAA21117); Cry8Ab1 (Accession #EU044830); Cry8Ac1 (Accession #KC156662); Cry8Ad1 (Accession #KC156684); Cry8Ba1 (Accession #AAA21118); Cry8Bb1 (Accession #CAD57542); Cry8Bc1 (Accession #CAD57543); Cry8Ca1 (Accession #AAA21119); Cry8Ca2 (Accession #AAR98783); Cry8Ca3 (Accession #EU625349); Cry8Ca4 (Accession #ADB54826); Cry8Da1 (Accession #BAC07226); Cry8Da2 (Accession #BD133574); Cry8Da3 (Accession #BD133575); Cry8Db1 (Accession #BAF93483); Cry8Ea1 (Accession #AAQ73470); Cry8Ea2 (Accession #EU047597); Cry8Ea3 (Accession #KC855216); Cry8Fa1 (Accession #AAT48690); Cry8Fa2 (Accession #HQ174208); Cry8Fa3 (Accession #AFH78109); Cry8Ga1 (Accession #AAT46073); Cry8Ga2 (Accession #ABC42043); Cry8Ga3 (Accession #FJ198072); Cry8Ha1 (Accession #AAW81032); Cry8Ia1 (Accession #EU381044); Cry8Ia2 (Accession #GU073381); Cry8Ia3 (Accession #HM044664); Cry8Ia4 (Accession #KC156674); Cry8Ib1 (Accession #GU325772); Cry81b2 (Accession #KC156677); Cry8Ja1 (Accession #EU625348); Cry8Ka1 (Accession #FJ422558); Cry8Ka2 (Accession #ACN87262); Cry8Kb1 (Accession #HM123758); Cry8Kb2 (Accession #KC156675); Cry8La1 (Accession #GU325771); Cry8Ma1 (Accession #HM044665); Cry8Ma2 (Accession #EEM86551); Cry8Ma3 (Accession #HM210574); Cry8Na1 (Accession #HM640939); Cry8Pa1 (Accession #HQ388415); Cry8Qa1 (Accession #HQ441166); Cry8Qa2 (Accession #KC152468); Cry8Ra1 (Accession #AFP87548); Cry8Sa1 (Accession #JQ740599); Cry8Ta1 (Accession #KC156673); Cry8-like (Accession #FJ770571); Cry8-like (Accession #ABS53003); Cry9Aa1 (Accession #CAA41122); Cry9Aa2 (Accession #CAA41425); Cry9Aa3 (Accession #GQ249293); Cry9Aa4 (Accession #GQ249294); Cry9Aa5 (Accession #JX174110); Cry9Aa like (Accession #AAQ52376); Cry9Bal (Accession #CAA52927); Cry9Ba2 (Accession #GU299522); Cry9Bb1 (Accession #AAV28716); Cry9Ca1 (Accession #CAA85764); Cry9Ca2 (Accession #AAQ52375); Cry9Da1 (Accession #BAA19948); Cry9Da2 (Accession #AAB97923); Cry9Da3 (Accession #GQ249293); Cry9Da4 (Accession #GQ249297); Cry9Db1 (Accession #AAX78439); Cry9Dc1 (Accession #KC156683); Cry9Ea1 (Accession #BAA34908); Cry9Ea2 (Accession #AAO12908); Cry9Ea3 (Accession #ABM21765); Cry9Ea4 (Accession #ACE88267); Cry9Ea5 (Accession #ACF04743); Cry9Ea6 (Accession #ACG63872); Cry9Ea7 (Accession #FJ380927); Cry9Ea8 (Accession #GQ249292); Cry9Ea9 (Accession #JN651495); Cry9Eb1 (Accession #CAC50780); Cry9Eb2 (Accession #GQ249298); Cry9Eb3 (Accession #KC156646); Cry9Ec1 (Accession #AAC63366); Cry9Ed1 (Accession #AAX78440); Cry9Ee1 (Accession #GQ249296); Cry9Ee2 (Accession #KC156664); Cry9Fa1 (Accession #KC156692); Cry9Ga1 (Accession #KC156699); Cry9-like (Accession #AAC63366); Cry10Aa1 (Accession #AAA22614); Cry10Aa2 (Accession #E00614); Cry10Aa3 (Accession #CAD30098); Cry10Aa4 (Accession #AFB18318); Cry10A-like (Accession #DQ167578); Cry11Aa1 (Accession #AAA22352); Cry11Aa2 (Accession #AAA22611); Cry11Aa3 (Accession #CAD30081); Cry11Aa4 (Accession #AFB18319); Cry11Aa-like (Accession #DQ166531); Cry11Ba1 (Accession #CAA60504); Cry11Bb1 (Accession #AAC97162); Cry11Bb2 (Accession #HM068615); Cry12Aa1 (Accession #AAA22355); Cry13Aa1 (Accession #AAA22356); Cry14Aa1 (Accession #AAA21516); Cry14Ab1 (Accession #KC156652); Cry15Aa1 (Accession #AAA22333); Cry16Aa1 (Accession #CAA63860); Cry17Aa1 (Accession #CAA67841); Cry18Aa1 (Accession #CAA67506); Cry18Ba1 (Accession #AAF89667); Cry18Ca1 (Accession #AAF89668); Cry19Aa1 (Accession #CAA68875); Cry19Ba1 (Accession #BAA32397); Cry19Ca1 (Accession #AFM37572); Cry20Aa1 (Accession #AAB93476); Cry20Ba1 (Accession #ACS93601); Cry20Ba2 (Accession #KC156694); Cry20-like (Accession #GQ144333); Cry21Aa1 (Accession #132932); Cry21Aa2 (Accession #I66477); Cry21Ba1 (Accession #BAC06484); Cry21Ca1 (Accession #JF521577); Cry21Ca2 (Accession #KC156687); Cry21Da1 (Accession #JF521578); Cry22Aa1 (Accession #134547); Cry22Aa2 (Accession #CAD43579); Cry22Aa3 (Accession #ACD93211); Cry22Ab1 (Accession #AAK50456); Cry22Ab2 (Accession #CAD43577); Cry22Ba1 (Accession #CAD43578); Cry22Bb1 (Accession #KC156672); Cry23Aa1 (Accession #AAF76375); Cry24Aa1 (Accession #AAC61891); Cry24Ba1 (Accession #BAD32657); Cry24Ca1 (Accession #CAJ43600); Cry25Aa1 (Accession #AAC61892); Cry26Aa1 (Accession #AAD25075); Cry27Aa1 (Accession #BAA82796); Cry28Aa1 (Accession #AAD24189); Cry28Aa2 (Accession #AAG00235); Cry29Aa1 (Accession #CAC80985); Cry30Aa1 (Accession #CAC80986); Cry30Ba1 (Accession #BAD00052); Cry30Ca1 (Accession #BAD67157); Cry30Ca2 (Accession #ACU24781); Cry30Da1 (Accession #EF095955); Cry30Db1 (Accession #BAE80088); Cry30Ea1 (Accession #ACC95445); Cry30Ea2 (Accession #FJ499389); Cry30Fa1 (Accession #ACI22625); Cry30Ga1 (Accession #ACG60020); Cry30Ga2 (Accession #HQ638217); Cry31Aa1 (Accession #BAB11757); Cry31Aa2 (Accession #AAL87458); Cry31Aa3 (Accession #BAE79808); Cry31Aa4 (Accession #BAF32571); Cry31Aa5 (Accession #BAF32572); Cry31Aa6 (Accession #BAI44026); Cry31Ab1 (Accession #BAE79809); Cry31Ab2 (Accession #BAF32570); Cry31Ac1 (Accession #BAF34368); Cry31Ac2 (Accession #AB731600); Cry31Ad1 (Accession #BAI44022); Cry32Aa1 (Accession #AAG36711); Cry32Aa2 (Accession #GU063849); Cry32Ab1 (Accession #GU063850); Cry32Ba1 (Accession #BAB78601); Cry32Ca1 (Accession #BAB78602); Cry32Cb1 (Accession #KC156708); Cry32Da1 (Accession #BAB78603); Cry32Ea1 (Accession #GU324274); Cry32Ea2 (Accession #KC156686); Cry32Eb1 (Accession #KC156663); Cry32Fa1 (Accession #KC156656); Cry32Ga1 (Accession #KC156657); Cry32Ha1 (Accession #KC156661); Cry32Hb1 (Accession #KC156666); Cry32Ia1 (Accession #KC156667); Cry32Ja1 (Accession #KC156685); Cry32Ka1 (Accession #KC156688); Cry32La1 (Accession #KC156689); Cry32Ma1 (Accession #KC156690); Cry32Mb1 (Accession #KC156704); Cry32Na1 (Accession #KC156691); Cry32Oa1 (Accession #KC156703); Cry32Pa1 (Accession #KC156705); Cry32Qa1 (Accession #KC156706); Cry32Ra1 (Accession #KC156707); Cry32Sa1 (Accession #KC156709); Cry32Ta1 (Accession #KC156710); Cry32Ua1 (Accession #KC156655); Cry33Aa1 (Accession #AAL26871); Cry34Aa1 (Accession #AAG50341); Cry34Aa2 (Accession #AAK64560); Cry34Aa3 (Accession #AAT29032); Cry34Aa4 (Accession #AAT29030); Cry34Ab1 (Accession #AAG41671); Cry34Ac1 (Accession #AAG50118); Cry34Ac2 (Accession #AAK64562); Cry34Ac3 (Accession #AAT29029); Cry34Ba1 (Accession #AAK64565); Cry34Ba2 (Accession #AAT29033); Cry34Ba3 (Accession #AAT29031); Cry35Aa1 (Accession #AAG50342); Cry35Aa2 (Accession #AAK64561); Cry35Aa3 (Accession #AAT29028); Cry35Aa4 (Accession #AAT29025); Cry35Ab1 (Accession #AAG41672); Cry35Ab2 (Accession #AAK64563); Cry35Ab3 (Accession #AY536891); Cry35Ac1 (Accession #AAG50117); Cry35Ba1 (Accession #AAK64566); Cry35Ba2 (Accession #AAT29027); Cry35Ba3 (Accession #AAT29026); Cry36Aa1 (Accession #AAK64558); Cry37Aa1 (Accession #AAF76376); Cry38Aa1 (Accession #AAK64559); Cry39Aa1 (Accession #BAB72016); Cry40Aa1 (Accession #BAB72018); Cry40Ba1 (Accession #BAC77648); Cry40Ca1 (Accession #EU381045); Cry40Da1 (Accession #ACF15199); Cry41Aa1 (Accession #BAD35157); Cry41Ab1 (Accession #BAD35163); Cry41Ba1 (Accession #HM461871); Cry41Ba2 (Accession #ZP_04099652); Cry42Aa1 (Accession #BAD35166); Cry43Aa1 (Accession #BAD15301); Cry43Aa2 (Accession #BAD95474); Cry43Ba1 (Accession #BAD15303); Cry43Ca1 (Accession #KC156676); Cry43Cb1 (Accession #KC156695); Cry43Cc1 (Accession #KC156696); Cry43-like (Accession #BAD15305); Cry44Aa (Accession #BAD08532); Cry45Aa (Accession #BAD22577); Cry46Aa (Accession #BAC79010); Cry46Aa2 (Accession #BAG68906); Cry46Ab (Accession #BAD35170); Cry47Aa (Accession #AAY24695); Cry48Aa (Accession #CAJ18351); Cry48Aa2 (Accession #CAJ86545); Cry48Aa3 (Accession #CAJ86546); Cry48Ab (Accession #CAJ86548); Cry48Ab2 (Accession #CAJ86549); Cry49Aa (Accession #CAH56541); Cry49Aa2 (Accession #CAJ86541); Cry49Aa3 (Accession #CAJ86543); Cry49Aa4 (Accession #CAJ86544); Cry49Ab1 (Accession #CAJ86542); Cry50Aa1 (Accession #BAE86999); Cry50Ba1 (Accession #GU446675); Cry50Ba2 (Accession #GU446676); Cry51Aa1 (Accession #ABI14444); Cry51Aa2 (Accession #GU570697); Cry52Aa1 (Accession #EF613489); Cry52Ba1 (Accession #FJ361760); Cry53Aa1 (Accession #EF633476); Cry53Ab1 (Accession #FJ361759); Cry54Aa1 (Accession #ACA52194); Cry54Aa2 (Accession #GQ140349); Cry54Ba1 (Accession #GU446677); Cry55Aa1 (Accession #ABW88932); Cry54Ab1 (Accession #JQ916908); Cry55Aa2 (Accession #AAE33526); Cry56Aa1 (Accession #ACU57499); Cry56Aa2 (Accession #GQ483512); Cry56Aa3 (Accession #JX025567); Cry57Aa1 (Accession #ANC87261); Cry58Aa1 (Accession #ANC87260); Cry59Ba1 (Accession #JN790647); Cry59Aa1 (Accession #ACR43758); Cry60Aa1 (Accession #ACU24782); Cry60Aa2 (Accession #EAO57254); Cry60Aa3 (Accession #EEM99278); Cry60Ba1 (Accession #GU810818); Cry60Ba2 (Accession #EAO57253); Cry60Ba3 (Accession #EEM99279); Cry61Aa1 (Accession #HM035087); Cry61Aa2 (Accession #HM132125); Cry61Aa3 (Accession #EEM19308); Cry62Aa1 (Accession #HM054509); Cry63Aa1 (Accession #BAI44028); Cry64Aa1 (Accession #BAJ05397); Cry65Aa1 (Accession #HM461868); Cry65Aa2 (Accession #ZP_04123838); Cry66Aa1 (Accession #HM485581); Cry66Aa2 (Accession #ZP_04099945); Cry67Aa1 (Accession #HM485582); Cry67Aa2 (Accession #ZP_04148882); Cry68Aa1 (Accession #HQ113114); Cry69Aa1 (Accession #HQ401006); Cry69Aa2 (Accession #JQ821388); Cry69Ab1 (Accession #JN209957); Cry70Aa1 (Accession #JN646781); Cry70Ba1 (Accession #ADO51070); Cry70Bb1 (Accession #EEL67276); Cry71Aa1 (Accession #JX025568); Cry72Aa1 (Accession #JX025569).

Examples of δ-endotoxins also include but are not limited to Cry1A proteins of U.S. Pat. Nos. 5,880,275 and 7,858,849; a DIG-3 or DIG-11 toxin (N-terminal deletion of α-helix 1 and/or α-helix 2 variants of Cry proteins such as Cry1A) of U.S. Pat. Nos. 8,304,604 and 8.304,605, Cry1B of U.S. patent application Ser. No. 10/525,318; Cry1C of U.S. Pat. No. 6,033,874; Cry1F of U.S. Pat. Nos. 5,188,960, 6,218, 188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry 15 protein of Naimov, et al., (2008) Applied and Environmental Microbiology 74:7145-7151; a Cry22, a Cry34Ab1 protein of U.S. Pat. Nos. 6,127,180, 6,624, 145 and 6,340,593; a CryET33 and CryET34 protein of U.S. Pat. Nos. 6,248,535, 6,326,351, 6,399,330, 6,949,626, 7,385, 107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; TIC3131, TIC 3400, and TIC3407 of US Patent Application Publication Number 2015/0047076; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of U.S. Pat. No. 20,110,023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of U.S. Pat. No. 20,090,144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082,AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; and Cry proteins such as Cry1A and Cry3A having modified proteolytic sites of U.S. Pat. No. 8,319,019; and a Cry1Ac, Cry2Aa and Cry1Ca toxin protein from Bacillus thuringiensis strain VBTS 2528 of US Patent Application Publication Number 2011/0064710. Other Cry proteins are well known to one skilled in the art (see, Crickmore, et al., “Bacillus thuringiensis toxin nomenclature” (2011), at lifesci.sussex.ac.uk/home/Neil_Crickmore/Bt/which can be accessed on the world-wide web using the “www” prefix). The insecticidal activity of Cry proteins is well known to one skilled in the art (for review, see, van Frannkenhuyzen, (2009) J. Invert. Path. 101:1-16). The use of Cry proteins as transgenic plant traits is well known to one skilled in the art and Cry-transgenic plants including but not limited to Cry1Ac, Cry1Ac+Cry2Ab, Cry1Ab, Cry1A.105, Cry1F, Cry1Fa2, Cry1F+Cry1Ac, Cry2Ab, Cry3A, mCry3A, Cry3Bb1, Cry34Ab1, Cry35Ab1, Vip3A, mCry3A, Cry9c and CBI-Bt have received regulatory approval (see, Sanahuja, (2011) Plant Biotech Journal 9:283-300 and the CERA (2010) GM Crop Database Center for Environmental Risk Assessment (CERA), ILSI Research Foundation, Washington D.C. at cera-gmc.org/index.php?action=gm_crop_database which can be accessed on the world-wide web using the “www” prefix). More than one pesticidal proteins well known to one skilled in the art can also be expressed in plants such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605)); Cry34Ab/35Ab and Cry6Aa (U.S. Pat. No. 20,130,167269); Cry34Ab/VCry35Ab & Cry3Aa (U.S. Pat. No. 20,130,167268); Cry3A and Cry1Ab or Vip3Aa (U.S. Pat. No. 20,130,116170); and Cry1F, Cry34Ab1, and Cry35Ab1 (PCT/US2010/060818). Pesticidal proteins also include insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413). Pesticidal proteins also include VIP (vegetative insecticidal proteins) toxins of U.S. Pat. Nos. 5,877,012, 6,107,279, 6,137,033, 7,244,820, 7,615,686, and 8,237,020, and the like. Other VIP proteins are well known to one skilled in the art (see, lifesci.sussex.ac.uk/home/Neil_Crickmore/Bt/vip.html which can be accessed on the world-wide web using the “www” prefix). Pesticidal proteins also include toxin complex (TC) proteins, obtainable from organisms such as Xenorhabdus, Photorhabdus and Paenibacillus (see, U.S. Pat. Nos. 7,491,698 and 8,084,418). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366).

(C) An insect-specific hormone or pheromone such as an ecdysteroid and juvenile hormone, a variant thereof, a mimetic based thereon, or an antagonist or agonist thereof. See, for example, the disclosure by Hammock, et al., (1990) Nature 344:458, of baculovirus expression of cloned juvenile hormone esterase, an inactivator of juvenile hormone.

(D) An insect-specific peptide which, upon expression, disrupts the physiology of the affected pest. For example, see the disclosures of Regan, (1994) J. Biol. Chem. 269:9 (expression cloning yields DNA coding for insect diuretic hormone receptor); Pratt, et al., (1989) Biochem. Biophys. Res. Comm. 163:1243 (an allostatin is identified in Diploptera puntata); Chattopadhyay, et al., (2004) Critical Reviews in Microbiology 30(1): 33-54; Zjawiony, (2004) J Nat Prod 67(2): 300-310; Carlini and Grossi-de-Sa, (2002) Toxicon 40(11): 1515-1539; Ussuf, et al., (2001) Curr Sci. 80(7): 847-853 and Vasconcelos and Oliveira, (2004) Toxicon 44(4): 385-403. See also, U.S. Pat. No. 5,266,317 to Tomalski, et al., who disclose genes encoding insect-specific toxins.

(E) An enzyme responsible for a hyperaccumulation of a monterpene, a sesquiterpene, a steroid, hydroxamic acid, a phenylpropanoid derivative or another non-protein molecule with insecticidal activity.

(F) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See, PCT Application Number WO 93/02197 in the name of Scott, et al., which discloses the nucleotide sequence of a callase gene. DNA molecules which contain chitinase-encoding sequences can be obtained, for example, from the ATCC under Accession Numbers 39637 and 67152. See also, Kramer, et al., (1993) Insect Biochem. Molec. Biol. 23:691, who teach the nucleotide sequence of a cDNA encoding tobacco hookworm chitinase, and Kawalleck, et al., (1993) Plant Molec. Biol. 21:673, who provide the nucleotide sequence of the parsley ubi4-2 polyubiquitin gene, U.S. patents application Ser. Nos. 10/389,432, 10/692,367 and U.S. Pat. No. 6,563,020.

(G) A molecule that stimulates signal transduction. For example, see the disclosure by Botella, et al., (1994) Plant Molec. Biol. 24:757, of nucleotide sequences for mung bean calmodulin cDNA clones, and Griess, et al., (1994) Plant Physiol. 104:1467, who provide the nucleotide sequence of a maize calmodulin cDNA clone.

(H) A hydrophobic moment peptide. See, PCT Application Number WO 95/16776 and U.S. Pat. No. 5,580,852 (disclosure of peptide derivatives of Tachyplesin which inhibit fungal plant pathogens) and PCT Application Number WO 95/18855 and U.S. Pat. No. 5,607,914) (teaches synthetic antimicrobial peptides that confer disease resistance).

(I) A membrane permease, a channel former or a channel blocker. For example, see the disclosure by Jaynes, et al., (1993) Plant Sci. 89:43, of heterologous expression of a cecropin-beta lytic peptide analog to render transgenic tobacco plants resistant to Pseudomonas solanacearum.

(J) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., (1990) Ann. Rev. Phytopathol. 28:451. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(K) An insect-specific antibody or an immunotoxin derived therefrom. Thus, an antibody targeted to a critical metabolic function in the insect gut would inactivate an affected enzyme, killing the insect. Cf. Taylor, et al., Abstract #497, SEVENTH INT'L SYMPOSIUM ON MOLECULAR PLANT-MICROBE INTERACTIONS (Edinburgh, Scotland, 1994) (enzymatic inactivation in transgenic tobacco via production of single-chain antibody fragments).

(L) A virus-specific antibody. See, for example, Tavladoraki, et al., (1993) Nature 366:469, who show that transgenic plants expressing recombinant antibody genes are protected from virus attack.

(M) A developmental-arrestive protein produced in nature by a pathogen or a parasite. Thus, fungal endo alpha-1,4-D-polygalacturonases facilitate fungal colonization and plant nutrient release by solubilizing plant cell wall homo-alpha-1,4-D-galacturonase. See, Lamb, et al., (1992) Bio/Technology 10:1436. The cloning and characterization of a gene which encodes a bean endopolygalacturonase-inhibiting protein is described by Toubart, et al., (1992) Plant J. 2:367.

(N) A developmental-arrestive protein produced in nature by a plant. For example, Logemann, et al., (1992) Bio/Technology 10:305, have shown that transgenic plants expressing the barley ribosome-inactivating gene have an increased resistance to fungal disease.

(O) Genes involved in the Systemic Acquired Resistance (SAR) Response and/or the pathogenesis related genes. Briggs, (1995) Current Biology 5(2): 128-131, Pieterse and Van Loon, (2004) Curr. Opin. Plant Bio. 7(4): 456-64 and Somssich, (2003) Cell 113(7): 815-6.

(P) Antifungal genes (Cornelissen and Melchers, (1993) Pl. Physiol. 101:709-712 and Parijs, et al., (1991) Planta 183:258-264 and Bushnell, et al., (1998) Can. J. of Plant Path. 20(2): 137-149. Also see, U.S. patent application Ser. No. 09/950,933.

(Q) Detoxification genes, such as for fumonisin, beauvericin, moniliformin and zearalenone and their structurally related derivatives. For example, see, U.S. Pat. No. 5,792,931.

(R) Cystatin and cysteine proteinase inhibitors. See, U.S. application Ser. Number 10/947,979.

(S) Defensin genes. See, WO03/000863 and U.S. application Ser. Number 10/178,213.

(T) Genes conferring resistance to nematodes. See, WO 03/033651 and Urwin, et. al., (1998) Planta 204:472-479, Williamson (1999) Curr Opin Plant Bio. 2(4): 327-31.

(U) Genes such as rcg1conferring resistance to Anthracnose stalk rot, which is caused by the fungus Colletotrichum graminiola. See, Jung, et al., Generation-means analysis and quantitative trait locus mapping of Anthracnose Stalk Rot genes in Maize, Theor. Appl. Genet. (1994) 89:413-418, as well as, U.S. Provisional Patent Application No. 60/675,664.

(V) Nucleic Acids that relate to down-regulation of expression of target genes in insect pest species by interfering ribonucleic acid (RNA) molecules, which control the insect pest species. PCT Publication WO 2011/025860 and WO 2014/153254 describe methods of inhibiting expression of target genes in invertebrate plant pests including Diabrotica plant pests. Furthermore, PCT Publication WO 2009/091864 describes compositions and methods for the suppression of target genes from insect pest species including pests from the Lygus genus.

2. Transgenes That Confer Resistance To A Herbicide, For Example:

(A) A herbicide that inhibits the growing point or meristem, such as an imidazolinone or a sulfonylurea. Exemplary genes in this category code for mutant ALS and AHAS enzyme as described, for example, by Lee, et al., (1988) EMBO J. 7:1241 and Miki, et al., (1990) Theor. Appl. Genet. 80:449, respectively. See also, U.S. Pat. Nos. 5,605,011; 5,013,659; 5,141,870; 5,767,361; 5,731,180; 5,304,732; 4,761,373; 5,331,107; 5,928,937 and 5,378,824 and international publication WO 96/33270.

(B) Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes) and pyridinoxy or phenoxy proprionic acids and cycloshexones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and international publications EP1173580; WO 01/66704; EP1173581 and EP1173582. Glyphosate resistance is also imparted to plants that express a gene that encodes a glyphosate oxido-reductase enzyme as described more fully in U.S. Pat. Nos. 5,776,760 and 5,463,175, which are incorporated herein by reference in their entirety. In addition glyphosate resistance can be imparted to plants by the over expression of genes encoding glyphosate N-acetyltransferase. See, for example, U.S. patents application Ser. Nos. 11/405,845 and 10/427,692 and PCT Application Number US01/46227. A DNA molecule encoding a mutant aroA gene can be obtained under ATCC Accession Number 39256 and the nucleotide sequence of the mutant gene is disclosed in U.S. Pat. No. 4,769,061 to Comai. EP Patent Application Number 0 333 033 to Kumada, et al., and U.S. Pat. No. 4,975,374 to Goodman, et al., disclose nucleotide sequences of glutamine synthetase genes which confer resistance to herbicides such as L-phosphinothricin. The nucleotide sequence of a phosphinothricin-acetyl-transferase gene is provided in EP Patent Numbers 0 242 246 and 0 242 236 to Leemans, et al., De Greef, et al., (1989) Bio/Technology 7:61 which describe the production of transgenic plants that express chimeric bar genes coding for phosphinothricin acetyl transferase activity. See also, U.S. Pat. Nos. 5,969,213; 5,489,520; 5,550,318; 5,874,265; 5,919,675; 5,561,236; 5,648,477; 5,646,024; 6,177,616 B1 and 5,879,903. Exemplary genes conferring resistance to phenoxy proprionic acids and cycloshexones, such as sethoxydim and haloxyfop, are the Acc1-S1, Acc1-S2 and Acc1-S3 genes described by Marshall, et al., (1992) Theor. Appl. Genet. 83:435.

(C) A herbicide that inhibits photosynthesis, such as a triazine (psbA and gs+genes) and a benzonitrile (nitrilase gene). Przibilla, et al., (1991) Plant Cell 3:169, describe the transformation of Chlamydomonas with plasmids encoding mutant psbA genes. Nucleotide sequences for nitrilase genes are disclosed in U.S. Pat. No. 4,810,648 to Stalker, herein incorporated by reference in its entirety, and DNA molecules containing these genes are available under ATCC Accession Numbers 53435, 67441 and 67442. Cloning and expression of DNA coding for a glutathione S-transferase is described by Hayes, et al., (1992) Biochem. J. 285:173.

(D) Acetohydroxy acid synthase, which has been found to make plants that express this enzyme resistant to multiple types of herbicides, has been introduced into a variety of plants (see, e.g., Hattori, et al., (1995) Mol Gen Genet 246:419). Other genes that confer resistance to herbicides include: a gene encoding a chimeric protein of rat cytochrome P4507A1 and yeast NADPH-cytochrome P450 oxidoreductase (Shiota, et al., (1994) Plant Physiol. 106(1): 17-23), genes for glutathione reductase and superoxide dismutase (Aono, et al., (1995) Plant Cell Physiol 36:1687, and genes for various phosphotransferases (Datta, et al., (1992) Plant Mol Biol 20:619).

(E) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1 and 5,767,373; and international publication number WO 01/12825.

3. Transgenes That Confer Or Contribute To an Altered Grain Characteristic, Such As:
(A) Altered fatty acids, for example, by

(1) Down-regulation of stearoyl-ACP desaturase to increase stearic acid content of the plant. See, Knultzon, et al., (1992) Proc. Natl. Acad. Sci. USA 89:2624 and WO99/64579 (Genes for Desaturases to Alter Lipid Profiles in Corn),

(2) Elevating oleic acid via FAD-2 gene modification and/or decreasing linolenic acid via FAD-3 gene modification (see, U.S. Pat. Nos. 6,063,947; 6,323,392; 6,372,965 and WO 93/11245),

(3) Altering conjugated linolenic or linoleic acid content, such as in WO 01/12800,

(4) Altering LEC1, AGP, Dek1, Superal1, milps, various lpa genes such as lpa1, lpa3, hpt or hggt. For example, see, WO 02/42424, WO 98/22604, WO 03/011015, U.S. Pat. Nos. 6,423,886, 6,197,561, 6,825,397, US Patent Application Publication Numbers 2003/0079247, 2003/0204870, WO02/057439, WO03/011015 and Rivera-Madrid, et. al., (1995) Proc. Natl. Acad. Sci. 92:5620-5624.

(B) Altered phosphorus content, for example, by the

(1) Introduction of a phytase-encoding gene would enhance breakdown of phytate, adding more free phosphate to the transformed plant. For example, see, Van Hartingsveldt, et al., (1993) Gene 127:87, for a disclosure of the nucleotide sequence of an Aspergillus niger phytase gene.

(2) Up-regulation of a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy, et al., (1990) Maydica 35:383 and/or by altering inositol kinase activity as in WO 02/059324, US Patent Application Publication Number 2003/0009011, WO 03/027243, US Patent Application Publication Number 2003/0079247, WO 99/05298, U.S. Pat. No. 6,197,561, U.S. Pat. No. 6,291,224, U.S. Pat. No. 6,391,348,WO2002/059324, US Patent Application Publication Number 2003/0079247, WO98/45448, WO99/55882, WO01/04147.

(C) Altered carbohydrates effected, for example, by altering a gene for an enzyme that affects the branching pattern of starch or a gene altering thioredoxin such as NTR and/or TRX (see, U.S. Pat. No. 6,531,648) and/or a gamma zein knock out or mutant such as cs27 or TUSC27 or en27 (see, U.S. Pat. No. 6,858,778 and US Patent Application Publication Numbers 2005/0160488 and 2005/0204418). See, Shiroza, et al., (1988) J. Bacteriol. 170:810 (nucleotide sequence of Streptococcus mutans fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec. Biol. 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol. 102:1045 (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

(D) Altered antioxidant content or composition, such as alteration of tocopherol or tocotrienols. For example, see U.S. Pat. No. 6,787,683, US Patent Application Publication Number 2004/0034886 and WO 00/68393 involving the manipulation of antioxidant levels through alteration of a phytl prenyl transferase (ppt), WO 03/082899 through alteration of a homogentisate geranyl geranyl transferase (hggt).

(E) Altered essential seed amino acids. For example, see U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US Patent Application Publication Number 2003/0163838, US Patent Application Publication Number 2003/0150014, US Patent Application Publication Number 2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US Patent Application Publication Number 2004/0025203 (UDPGdH), U.S. Pat. No. 6,194,638 (RGP).

### 4. Genes that Control Male-sterility

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant and thus creating a plant that is male sterile because the inducible promoter is not “on” resulting in the male fertility gene not being transcribed. Fertility is restored by inducing, or turning “on”, the promoter, which in turn allows the gene conferring male fertility to be transcribed.

(A) Introduction of a deacetylase gene under the control of a tapetum-specific promoter and with the application of the chemical N-Ac-PPT (WO 01/29237).

(B) Introduction of various stamen-specific promoters (WO 92/13956, WO 92/13957).

(C) Introduction of the barnase and the barstar gene (Paul, et al., (1992) Plant Mol. Biol. 19:611-622).

For additional examples of nuclear male and female sterility systems and genes, see also, U.S. Pat. Nos. 5,859,341; 6,297,426; 5,478,369; 5,824,524; 5,850,014 and 6,265,640.

5. Genes that create a site for site specific DNA integration

This includes the introduction of FRT sites that may be used in the FLP/FRT system and/or Lox sites that may be used in the Cre/Loxp system. For example, see Lyznik, et al., (2003) Plant Cell Rep 21:925-932 and WO 99/25821. Other systems that may be used include the Gin recombinase of phage Mu (Maeser, et al., 1991; Vicki Chandler, The Maize Handbook ch. 118 (Springer-Verlag 1994), the Pin recombinase of E. coli (Enomoto, et al., 1983), and the R/RS system of the pSR1 plasmid (Araki, et al., 1992).

6. Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress. For example, see, WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521, and WO9938977 describing genes, including CBF genes and transcription factors effective in mitigating the negative effects of freezing, high salinity, and drought on plants, as well as conferring other positive effects on plant phenotype; US Patent Application Publication Number 2004/0148654 and WO01/36596 where abscisic acid is altered in plants resulting in improved plant phenotype such as increased yield and/or increased tolerance to abiotic stress; WO2000/006341, WO04/090143, U.S. patent application Ser. No. 10/817,483 and U.S. Pat. No. 6,992,237, where cytokinin expression is modified resulting in plants with increased stress tolerance, such as drought tolerance, and/or increased yield, herein incorporated by reference in their entirety. Also see WO0202776, WO2003052063, JP2002281975, U.S. Pat. No. 6,084,153, WO0164898, U.S. Pat. No. 6,177,275 and U.S. Pat. No. 6,107,547 (enhancement of nitrogen utilization and altered nitrogen responsiveness). For ethylene alteration, see US Patent Application Publication Number 2004/0128719, US Patent Application Publication Number 2003/0166197 and WO200032761. For plant transcription factors or transcriptional regulators of abiotic stress, see, e.g., US Patent Application Publication Number 2004/0098764 or US Patent Application Publication Number 2004/0078852.

Other genes and transcription factors that affect plant growth and agronomic traits such as yield, flowering, plant growth and/or plant structure, can be introduced or introgressed into plants, see, e.g., WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. Nos. 6,794,560, 6,307,126 (GAI), WO99/09174 (D8 and Rht) and WO2004076638 and WO2004031349 (transcription factors).

“RNAi” refers to a series of related techniques to reduce the expression of genes (see, for example, U.S. Pat. No. 6,506,559). These include “antisense inhibition,” the production of antisense RNA transcripts capable of suppressing the expression of the target protein, and “co-suppression” or “sense-suppression,” which refer to the production of sense RNA transcripts capable of suppressing the expression of identical or substantially similar foreign or endogenous genes (U.S. Pat. No. 5,231,020). Such techniques rely on the use of constructs resulting in the accumulation of double stranded RNA with one strand complementary to the target gene to be silenced. The regulatory sequences disclosed herein may be used to drive expression of constructs that will result in RNA interference including microRNAs and siRNAs.

Some embodiments relate to down-regulation of expression of target genes in insect pest species by interfering ribonucleic acid (RNA) molecules. PCT Publication WO 2007/074405 describes methods of inhibiting expression of target genes in invertebrate pests including Colorado potato beetle. PCT Publication WO 2005/110068 describes methods of inhibiting expression of target genes in invertebrate pests including in particular Western corn rootworm as a means to control insect infestation. Furthermore, PCT Publication WO 2009/091864 describes compositions and methods for the suppression of target genes from insect pest species including pests from the Lygus genus.

Nucleic acid molecules including silencing elements for targeting the vacuolar ATPase H subunit, useful for controlling a coleopteran pest population and infestation as described in US Patent Application Publication 2012/0198586. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the y-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+−ATPase G-subunit protein and an insect Tbp-1 such as Tat-binding protein. PCT publication WO 2007/035650 describes ribonucleic acid (RNA or double stranded RNA) that inhibits or down regulates the expression of a target gene that encodes Snf7. US Patent Application publication 2011/0054007 describes polynucleotide silencing elements targeting RPS10. US Patent Application publication 2014/0275208 and US2015/0257389 describes polynucleotide silencing elements targeting RyanR, HP2, and PAT3. US Patent Application publication 2011/0054007 describes polynucleotide silencing elements targeting RPS10. PCT publication WO 2016/205445 describes polynucleotide silencing elements that reduce fecundity, including NCLB, MAEL, BOULE, and VgR. PCT publications WO/2016/138106, WO 2016/060911, WO 2016/060912, WO 2016/060913, and WO 2016/060914 describe polynucleotide silencing elements targeting COPI coatomer subunit nucleic acid molecules that confer resistance to Coleopteran and Hemipteran pests. US Patent Application Publications 2012/029750, U.S. Pat. No. 20,120,297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EF1α Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubuliln Homologous Sequence.

The isolated regulatory element sequences disclosed herein may be modified to provide for a range of expression levels of the heterologous nucleotide sequence. Thus, less than the entire regulatory element region may be utilized and the ability to drive expression of the nucleotide sequence of interest retained. It is recognized that expression levels of the mRNA may be altered in different ways with deletions of portions of the promoter sequences. The mRNA expression levels may be decreased, or alternatively, expression may be increased as a result of regulatory element deletions if, for example, there is a negative regulatory element (for a repressor) that is removed during the truncation process. Generally, at least about 20 nucleotides of an isolated regulatory element sequence will be used to drive expression of a polynucleotide sequence.

Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639.

Expression cassettes comprising sequences disclosed herein may also contain at least one additional nucleotide sequence for a gene to be cotransformed into the organism. Alternatively, the additional sequence(s) can be provided on another expression cassette.

Where appropriate, the polynucleotide sequences whose expression is to be under the control of a hybrid regulatory element sequence of the present disclosure and any additional nucleotide sequence(s) may be optimized for increased expression in the transformed plant. That is, these nucleotide sequences can be synthesized using plant preferred codons for improved expression. See, for example, Campbell and Gowri, (1990) Plant Physiol. 92:1-11, for a discussion of host-preferred codon usage. Methods are available in the art for synthesizing plant-preferred genes. See, for example, Murray, et al., (1989) Nucleic Acids Res. 17:477-498.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous polynucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences may act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385). See, also, Della-Cioppa, et al., (1987) Plant Physiology 84:965-968. Methods known to enhance mRNA stability may also be utilized, for example, introns, such as the maize Ubiquitin intron (Christensen and Quail, (1996) Transgenic Res. 5:213-218; Christensen, et al., (1992) Plant Molecular Biology 18:675-689) or the maize AdhI intron (Kyozuka, et al., (1991) Mol. Gen. Genet. 228:40-48; Kyozuka, et al., (1990) Maydica 35:353-357) and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in expression cassettes. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330.

Selectable marker genes for selection of transformed cells or tissues may include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518).

Other genes that could serve utility in the recovery of transgenic events would include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15 (19): 8115 and Luehrsen, et al., (1992) Methods Enzymol. 216:397-414) and the maize genes encoding for anthocyanin production (Ludwig, et al., (1990) Science 247:449).

Expression cassette comprising a hybrid regulatory element operably linked to a polynucleotide sequence of interest may be used to transform any plant. In another embodiment, an expression cassette comprising the sequences of SEQ ID NOs: 1-74 operably linked to a polynucleotide sequence of interest may be used to transform any plant. In this manner, genetically modified plants, plant cells, plant tissue, seed, root and the like may be obtained.

Certain disclosed methods involve introducing a polynucleotide into a plant. As used herein, “introducing” is intended to mean presenting to the plant the polynucleotide in such a manner that the sequence gains access to the interior of a cell of the plant. The methods of the disclosure do not depend on a particular method for introducing a sequence into a plant, only that the polynucleotide gains access to the interior of at least one cell of the plant. Methods for introducing polynucleotide into plants are known in the art including, but not limited to, stable transformation methods, transient transformation methods and virus-mediated methods.

A “stable transformation” is a transformation in which the polynucleotide construct introduced into a plant integrates into the genome of the plant and is capable of being inherited by the progeny thereof. “Transient transformation” means that a polynucleotide is introduced into the plant and does not integrate into the genome of the plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P: 175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646;Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., and (1996) Nature Biotechnology 14:745-750 (maize via Agrobacterium tumefaciens).

In one embodiment, DNA constructs comprising a hybrid regulatory element may be provided to a plant using a variety of transient transformation methods. In another embodiment, DNA constructs comprising the disclosed sequences SEQ ID NOs: 1-74 may be provided to a plant using a variety of transient transformation methods. Such transient transformation methods include, but are not limited to, viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylimine (PEI; Sigma #P3143).

In other embodiments, a polynucleotide may be introduced into plants by contacting plants with a virus or viral nucleic acids. Generally, such methods involve incorporating a polynucleotide construct of the disclosure within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855 and WO99/25853. Briefly, the polynucleotide of the disclosure can be contained in transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide construct, for example, an expression cassette comprising one of SEQ ID NOs: 1-74, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the embodiments containing a desired polynucleotide is cultivated using methods well known to one skilled in the art.

The embodiments provide compositions for screening compounds that modulate expression within plants. The vectors, cells and plants can be used for screening candidate molecules for agonists and antagonists of the regulatory element sequences of SEQ ID NOs: 1-74. For example, a reporter gene can be operably linked to a regulatory element sequence and expressed as a transgene in a plant. Compounds to be tested are added and reporter gene expression is measured to determine the effect on promoter activity.

In one embodiment, a regulatory element, for example sequences SEQ ID NOs: 1-74 may be edited or inserted into a plant by genome editing using a CRISPR/Cas9 system.

In an aspect, the disclosed regulatory elements may be introduced into the genome of a plant using genome editing technologies, or previously introduced regulatory elements in the genome of a plant may be edited using genome editing technologies. For example, the disclosed regulatory elements may be introduced into a desired location in the genome of a plant through the use of double-stranded break technologies such as TALENs, meganucleases, zinc finger nucleases, CRISPR-Cas, and the like. For example, the disclosed regulatory elements may be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing regulatory elements of interest could be either an endogenous regulatory elements or a previously introduced regulatory elements.

In another aspect, where the disclosed regulatory element has previously been introduced into a genome, genome editing technologies may be used to alter or modify the introduced regulatory element sequence. Site specific modifications that can be introduced into the disclosed regulatory elements compositions include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced polynucleotide through the insertion, deletion or substitution of nucleotides within the introduced polynucleotide. Alternatively, double-stranded break technologies can be used to add additional nucleotide sequences to the introduced polynucleotide.

An “altered target site,” “altered target sequence.” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

All publications, patents and patent applications mentioned in the specification are indicative of the level of those skilled in the art to which this disclosure pertains. All publications, patents and patent applications are herein incorporated by reference to the same extent as if each individual publication, patent or patent application was specifically and individually indicated to be incorporated by reference.

The above description of various illustrated embodiments of the disclosure is not intended to be exhaustive or to limit the scope to the precise form disclosed. While specific embodiments of examples are described herein for illustrative purposes, various equivalent modifications are possible within the scope of the disclosure, as those skilled in the relevant art will recognize. The teachings provided herein can be applied to other purposes, other than the examples described above. Numerous modifications and variations are possible in light of the above teachings and, therefore, are within the scope of the appended claims.

These and other changes may be made in light of the above detailed description. In general, in the following claims, the terms used should not be construed to limit the scope to the specific embodiments disclosed in the specification and the claims.

Efforts have been made to ensure accuracy with respect to the numbers used (e.g. amounts, temperature, concentrations, etc.), but some experimental errors and deviations should be allowed for. Unless otherwise indicated, parts are parts by weight; molecular weight is average molecular weight; temperature is in degrees centigrade; and pressure is at or near atmospheric.

### EXPERIMENTAL

### Example 1: Root Hybrid Regulatory Elements

Roots are complex organs that can be divided into different regions; for example, the cap, meristem, elongation region and mature region. These regions reflect different stages of root cell identity and maturity. Genes in these cells are activated and inactivated and therefore may not express simultaneously in all regions of the root. Regulatory elements control the expression of these genes. Identifying naturally occurring regulatory elements with needed expression characteristics presents challenges at many different levels and can result in unanticipated expression levels and patterns in transgenic plants. An alternative to naturally occurring regulatory elements is a hybrid regulatory element that is created by combining segments of different regulatory elements. Segments of regulatory elements that function in different regions of the maize root can be combined, such that a hybrid regulatory element is created that functions in all or most regions of the root. Preferably, root specificity is maintained, although ectopic expression could occur.

### Example 2: Construction of Hybrid Root Regulatory Element Sequences

The hybrid regulatory sequences (SEQ ID NOs: 11-30 and 35-60) were each constructed using segments of regulatory elements that had been previously characterized for expression (Step 1; Table 1). The native regulatory elements were broadly categorized into two groups by expression pattern, root tip and mature region. The root tip included the cap, meristem, and the area of the elongation region adjacent to the meristem. The mature region included the mature region of the root and the portion of the elongation region adjacent to the mature region. The regulatory elements, belonging to either class, were evaluated for potential motifs using public and proprietary databases to aid in defining segments that may be used in the hybrid regulatory element (Step 2). Motifs algorithms useful for screening in the process are known in the art, such as Higo, K et al. (1998) Nucleic Acids Research. The process was initiated by assessing the position of motifs, the size and type of motifs as well as the clustering of motifs. Motif clustering between the TATA box and about 1kb upstream of the TATA box was favored as a segment to use in a hybrid promoter over segments that were more distal. The third step (Step 3) comprised assembly of chosen segments that contributed the 3′ end of the hybrid regulatory element, including the TATA box, and then the segments comprising the 5′ end of the hybrid regulatory element (Table 2). The assembly process generally placed the root tip segments at the 3′ end of the regulatory element. SEQ ID NOs: 11-18 were each cloned into a respective expression vector for introduction into maize plants. The β-glucuronidase (GUS) gene was used as a reporter gene to characterize expression directed by each of the hybrid regulatory elements. Approximately 10 maize transgenic events were regenerated for each vector.

TABLE 1
Expression Pattern of Root Promoters in Transgenic Maize Plants

| Parent | | Species | | | | |
|---|---|---|---|---|---|---|
| Regulatory | Fragment | of | | | Elongation | Mature |
| Element | category | origin | Cap | Meristem | region | region |
| SEQ ID | root tip | Bd | +++ | + | − | − |
| NO: 1 | | | | | | |
| SEQ ID | root tip | Bd | − | ++++ | + | − |
| NO: 2 | | | | | | |
| SEQ ID | root tip | Bd | − | ++ | + | − |
| NO: 3 | | | | | | |
| SEQ ID | root tip | Zm | − | +++ | ++ | − |
| NO: 4 | | | | | | |
| SEQ ID | mature region | Sb | − | − | ++ | ++ |
| NO: 5 | | | | | | |
| SEQ ID | mature region | Sb | − | − | +++ | +++ |
| NO: 6 | | | | | | |
| SEQ ID | mature region | Sb | − | + | − | ++ |
| NO: 7 | | | | | | |
| SEQ ID | mature region | Zm | − | − | + | +++ |
| NO: 8 | | | | | | |
| SEQ ID | mature region | Zm | − | − | + | +++ |
| NO: 9 | | | | | | |
| SEQ ID | mature region | Si | − | − | + | ++ |
| NO: 10 | | | | | | |

Based on histochemical staining
Zm = maize;
Sb = sorghum;
Bd = Brachypodium distachyon;
Si = Setaria italica

TABLE 2
Parent Regulatory Elements that Contribute Segments to the Root Hybrid Promoters

| Hybrid | | size of | | size of | | size of | | size of |
|---|---|---|---|---|---|---|---|---|
| Regulatory | | element 4 | | element 3 | | element 2 | Element 1 | element 1 |
| Element | Element 4 | (bp) | Element 3 | (bp) | Element 2 | (bp) | (TATA box) | (bp) |
| SEQ ID NO: 11 | SEQ ID NO: 8 | 973 | SEQ ID NO: 8 (Zm) | 973 | SEQ ID NO: 8 (Zm) | 1008 | SEQ ID NO: 4 (Zm) | 1152 |
| | (Zm) | | | | | | | |
| SEQ ID NO: 12 | SEQ ID NO: 6 | 1581 | SEQ ID NO: 6 (Sb) | 1581 | SEQ ID NO: 6 (Sb) | 1581 | SEQ ID NO: 4 (Zm) | 1152 |
| | (Sb) | | | | | | | |
| SEQ ID NO: 13 | | | SEQ ID NO: 8 (Zm) | 973 | SEQ ID NO: 6 (Sb) | 1229 | SEQ ID NO: 4 (Zm) | 587 |
| SEQ ID NO: 14 | | | SEQ ID NO: 6 (Sb) | 1581 | SEQ ID NO: 5 (Sb) | 441 | SEQ ID NO: 2 (Bd) | 1115 |
| SEQ ID NO: 15 | | | SEQ ID NO: 9 (Zm) | 781 | SEQ ID NO: 5 (Sb) | 441 | SEQ ID NO: 2 (Bd) | 1115 |
| SEQ ID NO: 16 | | | SEQ ID NO: 6 (Sb) | 1581 | SEQ ID NO: 5 (Sb) | 441 | SEQ ID NO: 1 (Bd) | 962 |
| SEQ ID NO: 17 | | | SEQ ID NO: 10 (Si) | 1172 | SEQ ID NO: 5 (Sb) | 1259 | SEQ ID NO: 3 (Bd) | 1551 |
| SEQ ID NO: 18 | | | SEQ ID NO: 10 (Si) | 1172 | SEQ ID NO: 7 (Sb) | 816 | SEQ ID NO: 3 (Bd) | 1551 |

Zm = maize;
Sb = sorghum;
Bd = Brachypodium distachyon;
Si = Setaria italica

### Example 3: Expression Analysis of Regulatory Elements in Transgenic Maize

Table 3 shows the expression result for each hybrid regulatory element in roots. Overall, each hybrid regulatory element drove expression and provided an expression pattern that was generally ubiquitous in the root. Expression was most consistent in the meristem, elongation region, and mature region between events. Expression in the cap was more variable as only one naturally occurring promoter (SEQ ID NO: 1; Table 1) explicitly drove expression in the cap. Therefore, expression in the cap was ectopic in the absence of SEQ ID NO: 1 in these experiments. Expression levels of the hybrid regulatory elements were generally higher than the individual parents by 2-fold or more; however, not all expressed at higher levels and were either equivalent to the highest expressing parent or slightly below.

SEQ ID NO: 11 was constructed using segments from parents with spatial expression characteristics that minimally overlapped (SEQ ID NO: 8 and SEQ ID NO: 4). Three copies of a segment from SEQ ID NO: 8 were fused to one copy of a segment from SEQ ID NO: 4. The results from histochemically stained transgenic maize roots showed expression was mostly in the meristem and mature region of transgenic roots, reflective of both parent regulatory segments. In higher expressing events, the expression pattern blended together for more uniform expression across the meristem, elongation and mature regions. Neither parent segment of SEQ ID NO: 11 drove expression in the cap; however, some events did display ectopic expression in the cap. An additional observation was expression in the stalk, which was exhibited by the parent regulatory element set forth in SEQ ID NO: 4.

SEQ ID NO: 12 demonstrates that segments from different species may act together in a hybrid format. SEQ ID NO: 6 is sourced from sorghum and drives expression at higher levels in the elongation region than SEQ ID NO: 8. Three copies of a parental regulatory element segment from SEQ ID NO: 6 were placed upstream of a parental regulatory element segment from SEQ ID NO: 4 in a configuration similar to SEQ ID NO: 11. The result of the construction was a more uniform expression pattern relative to SEQ ID NO: 11 in histochemically stained transgenic roots. Quantitative data showed the expression level directed by SEQ ID NO: 12 exceeded the level of the individual parent regulatory elements of SEQ ID NO: 6 and SEQ ID NO: 4, respectively. The increase was approximately 3-fold. Ectopic expression in the root cap was observed, and expression in stalk was observed as well.

SEQ ID NO: 13 eliminated repeated segments by combining one copy of each segment from SEQ ID NOs: 8, 6 and 4, respectively. Histochemical staining results showed uniform expression in the cap, meristem, elongation region and mature region. Histochemical staining is the result of immersing sampled tissue in a solution containing a substrate, X-Gluc (sodium salt), that results in the appearance of a blue precipitate at the site in the tissue where the B-glucuronidase enzyme is being expressed.

SEQ ID NOs: 14 and 15 were hybrid regulatory elements built with segments other than those from SEQ ID NO: 8, 6 and 4. In SEQ ID NO: 14, a segment from SEQ ID NO: 4 was replaced with a segment from SEQ ID NO: 2. SEQ ID NO: 2 has an expression pattern similar to SEQ ID NO: 4, but is from Brachypodium distachyon whereas SEQ ID NO: 4 is from maize. A segment from SEQ ID NO: 5 was added between segments from SEQ ID NO: 2 and SEQ ID NO: 6. SEQ ID NO: 15 consisted of segments from three species. SEQ ID NO: 9 originated from maize, SEQ ID NO: 5 was from sorghum, and SEQ ID NO: 2 was obtained from Brachypodium (Table 2). SEQ ID NO: 15 is very similar to SEQ ID NO: 14, with a difference of a SEQ ID NO: 6 segment substituted for a segment from SEQ ID NO: 9. Both SEQ ID NO: 14 and SEQ ID NO: 15 drove expression in maize roots (Table 3). Ectopic expression was observed in the cap. Other sites of expression included some SEQ ID NO: 14 and SEQ ID NO: 15 events with low levels of stalk and husk expression which differed from SEQ ID NO: 13 which had stalk and low levels of leaf expression.

SEQ ID NO: 16 demonstrates additional flexibility by replacing segments with similar expression patterns, such as SEQ ID NO: 2 and SEQ ID NO: 4, that were used for expression mostly in the meristem, with a segment from a parent regulatory element driving expression in a different part of the root tip. SEQ ID NO: 1 was selected for its root cap preference in transgenic maize plants and was used in place of SEQ ID NO: 2 in a SEQ ID NO: 14 configuration. SEQ ID NO: 16 drove expression in the cap and the other regions of the root (Table 3). Expression outside of roots was not detected.

SEQ ID NO: 17 explored using another regulatory element segment from Brachypodium, SEQ ID NO: 3, in place of SEQ ID NO: 2 and a segment from Setaria italica, SEQ ID NO: 10, to replace the maize segment, SEQ ID NO: 9.

SEQ ID NO: 18 was similar to SEQ ID NO: 17, with the exception that SEQ ID NO: 5 from sorghum was replaced with another segment from sorghum, SEQ ID NO: 7 (Table 2). Both SEQ ID NO: 17 and SEQ ID NO: 18 functioned in transgenic maize roots with SEQ ID NO: 17 producing more ectopic expression in the cap than SEQ ID NO: 18 (Table 3). A few events for SEQ ID NOs: 17 and 18 had some stalk expression.

The combined results from SEQ ID NOs: 11-18 demonstrate that segments from maize, sorghum, Brachypodium and Setaria italica that function in different regions of the maize root may be assembled to produce a hybrid regulatory element that functions in all regions of a maize root (Table 3).

TABLE 3
Expression Results for Each Hybrid Regulatory
Element in Transgenic Maize Roots

| Hybrid Regulatory | | | Elongation | Mature |
|---|---|---|---|---|
| Element | Cap | Meristem | region | region |
| Seq ID 11 | + | ++++ | ++ | +++ |
| Seq ID 12 | ++ | ++++ | ++++ | ++++ |
| Seq ID 13 | ++++ | ++++ | ++++ | ++++ |
| Seq ID 14 | ++++ | ++++ | ++++ | ++ |
| Seq ID 15 | ++ | ++++ | ++++ | ++ |
| Seq ID 16 | ++++ | +++ | ++++ | +++ |
| Seq ID 17 | ++++ | ++++ | ++++ | ++++ |
| Seq ID 18 | + | ++++ | ++ | +++ |

Based on histochemical staining

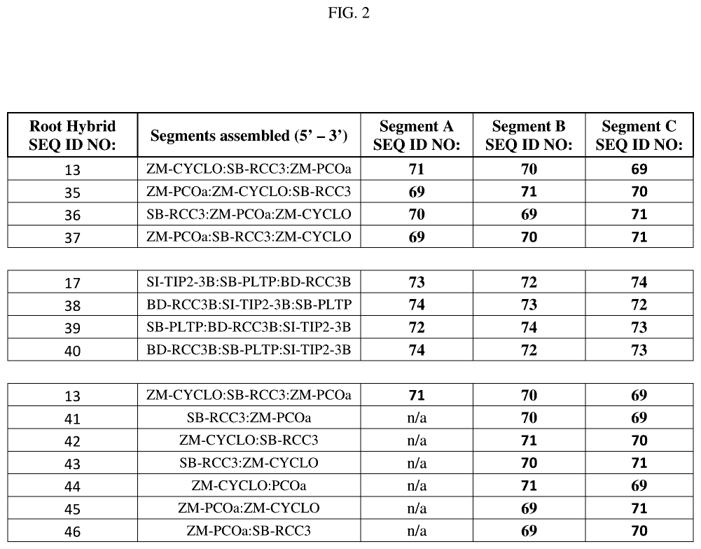

Additional segment combinations comprising the segment components of hybrid promoters, SEQ ID NOs: 13 and 17, were tested in maize plants. The three segments for each parental regulatory element were assigned a position with “A” indicating the 5′ segment, “B” the middle segment and “C” the 3′ segment (See FIG. 2). The segments were then put in re-ordered combinations as indicated in FIG. 2 and Tables 4 and 5 to test relative location of segments in a hybrid promoter. Altering the order of the three segments for SEQ ID NO: 13 resulted in parental-type expression for two of the three combinations (Table 4). The combination not showing parental-type expression had reduced expression in the root cap. These results indicate flexibility in the position of segments within the regulatory element, but that not all combinations will produce the same outcome. These results are supported by the reordering of SEQ ID NO: 17 which is comprised of different segments than SEQ ID NO: 13. Two of the three combinations resulted in parental type expression and like SEQ ID NO: 13, one did not (Table 5). The combination not showing the parental phenotype also had a different segment arrangement compared to SEQ ID NO: 13 (CBA versus CAB).

TABLE 4
Expression of Relative Orientation of Segments of a
Hybrid Regulatory Element in Transgenic Maize Roots

| Hybrid | | fragments | | | Elonga- | |
|---|---|---|---|---|---|---|
| Regulatory | | assembled | | | tion | Mature |
| Element | Order | (5′-3′) | Cap | Meristem | region | region |
| SEQ ID | ROOT | Mature, | ++++ | ++++ | ++++ | +++ |
| NO: 13 | HYBRID | mature- | | | | |
| | 2 (ABC) | elongation, | | | | |
| | | elongation- | | | | |
| | | mature | | | | |
| SEQ ID | CAB | Elongation- | ++++ | ++++ | ++++ | +++ |
| NO: 35 | | meristem, | | | | |
| | | mature, | | | | |
| | | mature- | | | | |
| | | elongation | | | | |
| SEQ ID | BCA | Mature- | ++++ | ++++ | ++++ | +++ |
| NO: 36 | | elongation, | | | | |
| | | elongation- | | | | |
| | | meristem, | | | | |
| | | mature | | | | |
| SEQ ID | CBA | Elongation- | + | ++++ | ++++ | +++ |
| NO: 37 | | meristem, | | | | |
| | | mature- | | | | |
| | | elongation, | | | | |
| | | mature | | | | |

Based on histochemical staining

TABLE 5
Expression of Relative Orientation of Segments of a
Hybrid Regulatory Element in Transgenic Maize Roots

| Hybrid | | fragments | | | Elonga- | |
|---|---|---|---|---|---|---|
| Regulatory | | assembled | | | tion | Mature |
| Element | Order | (5′-3′) | Cap | Meristem | region | region |
| SEQ ID | ROOT | Mature, | ++++ | ++++ | ++++ | +++ |
| NO: 17 | HYBRID | mature- | | | | |
| | 4 | elongation, | | | | |
| | | meristem- | | | | |
| | | elongation | | | | |
| SEQ ID | CAB (4) | Meristem- | + | ++++ | +++ | +++ |
| NO: 38 | | elongation, | | | | |
| | | mature, | | | | |
| | | mature- | | | | |
| | | elongation | | | | |
| SEQ ID | BCA (4) | Elongation- | ++++ | ++++ | ++++ | +++ |
| NO: 39 | | mature, | | | | |
| | | meristem- | | | | |
| | | elongation, | | | | |
| | | mature | | | | |
| SEQ ID | CBA (4) | Meristem- | ++++ | ++++ | ++++ | ++++ |
| NO: 40 | | elongation, | | | | |
| | | elongation- | | | | |
| | | mature, | | | | |
| | | mature | | | | |

In order to test the use of only two segments of parental regulatory clements to produce a unique expression pattern in a hybrid promoter, pairwise combinations using two segments of SEQ ID NO: 13 were made and compared back to the expression of SEQ ID NO: 13 (Table 6). Results showed combinations could be made that reflected SEQ ID NO: 13 expression, but others did not. Of those that did not, one combination (CA) did not entirely reflect the contribution of each segment. The results indicate that two segments are sufficient to produce a promoter that functions across different regions of the root, but the segments put together and how they are put together may impact the expression result.

TABLE 6
Expression of Relative Orientation of Segments of a
Hybrid Regulatory Element in Transgenic Maize Roots

| Hybrid | | fragments | | | Elonga- | |
|---|---|---|---|---|---|---|
| Regulatory | | assembled | | | tion | Mature |
| Element | Order | (5′-3′) | Cap | Meristem | region | region |
| SEQ ID | ROOT | Mature, | ++++ | ++++ | ++++ | +++ |
| NO: 13 | HYBRID | mature- | | | | |
| | 2 (ABC) | elongation, | | | | |
| | | elongation- | | | | |
| | | mature | | | | |
| SEQ ID | BC | Mature- | ++++ | ++++ | ++++ | +++ |
| NO: 41 | | elongation, | | | | |
| | | elongation- | | | | |
| | | meristem | | | | |
| SEQ ID | AB | Mature, | + | ++++ | ++++ | +++ |
| NO: 42 | | mature- | | | | |
| | | elongation | | | | |
| SEQ ID | BA | Mature- | − | ++++ | ++++ | +++ |
| NO: 43 | | elongation, | | | | |
| | | mature | | | | |
| SEQ ID | AC | Mature, | + | ++++ | +++ | ++ |
| NO: 44 | | elongation- | | | | |
| | | meristem | | | | |
| SEQ ID | CA | Elongation- | ++ | ++ | + | + |
| NO: 45 | | meristem, | | | | |
| | | mature | | | | |
| SEQ ID | CB | Elongation- | +++ | ++++ | ++++ | +++ |
| NO: 46 | | meristem, | | | | |
| | | mature- | | | | |
| | | elongation | | | | |

### Example 4: Hybrid Aerial Regulatory Elements

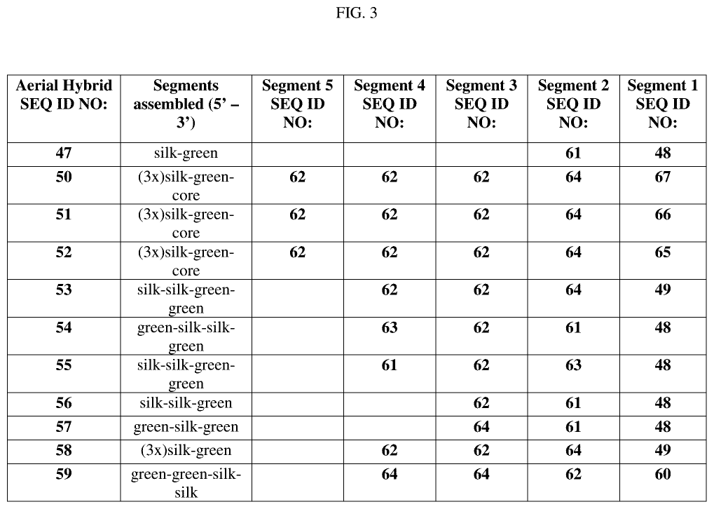

The methods used to create the hybrid root regulatory elements may also be applied to other hybrid regulatory element types, including above ground tissue expressing hybrid regulatory elements (“aerial regulatory elements”). Segments from a maize silk-preferred parent regulatory element (SEQ ID NO: 48) and a Brachypodium green tissue parent regulatory element (SEQ ID NO: 49) are combined, as described in Example 3 for SEQ ID NO: 11, where repeated segments from a silk-preferred regulatory element are placed upstream of a green tissue segment that supply a TATA box for SEQ ID NO: 48. The reverse was also performed where a silk-expressed segment from the maize silk-preferred parent regulatory element (SEQ ID NO: 48) provides the TATA box and multiple segments of the Brachypodium green tissue parent regulatory element (SEQ ID NO: 49) are upstream for an aerial hybrid regulatory element. The outcome of combing these elements, shown in Table 7 and FIG. 3, were that the resulting hybrid regulatory element drove transgene expression in both the leaves and silks of maize plants. Expression in other tissues such as root and stalk were not significantly different compared to the parents. Taken together, vegetative green tissue promoter segments can be combined with reproductive non-green tissue segments to produce a desired expression pattern in maize. The segments from parent regulatory elements may originate from different species.

Combining the segments was not necessarily expected to increase expression in the tissues of interest much beyond the level provided by the parents. So, to help modulate expression in the direction of increased levels, the −20 regions of three different plant viral promoters were used in replace of the −20 region of the segment providing the TATA box for aerial hybrid regulatory elements. Viral promoters are generally strong promoters in plants, so the −20 regions of these promoters, which consist of the TATA box region and other sequences that interact with the basal transcription machinery, may be viewed as “optimized” for expression. Therefore, linking upstream regulatory segments to these “optimized” sequence elements may result in increased expression levels of the hybrid regulatory element. The three viral promoters selected to provide the −20 regions were BYDV (Bean Yellow Dwarf Virus, SEQ ID NO: 67, HLCV (Hollyhock Leaf Crumple Virus, SEQ ID NO: 66 and Banana Streak Virus (AY), SEQ ID NO: 65. These represent the Geminiviridae and Caulimoviridae virus families, although other virus promoters and promoters from other families than the Geminiviridae and Caulimoviridae families may be used. Table 7 shows each of the −20 regions positively affected expression in leaves and silk. Expression in roots and stalk was not impacted the same way. The BSV(AY) −20 region provided the most significant increase in expression which included an increase in husk expression.

TABLE 7
Geminiviridae and Caulimoviridae Family −20 Regions
Positively Affected Expression in Leaves and Silk

| Upstream | | | | Root | | | |
|---|---|---|---|---|---|---|---|
| Activation | | | Root | (mature | | | |
| Element | Base | Leaf | (tip) | region) | Stalk | Silk | Husk |
| SEQ ID NO: | BD-FBPA | 10 | <0.1 | <0.1 | 1 | 1 | 3 |
| 49 | PRO | | | | | | |
| SEQ ID NO: | ZM-OEBF | <0.1 | <0.1 | <0.1 | <0.1 | 5 | <0.1 |
| 60 | PRO | | | | | | |
| SEQ ID NO: | BD-FBPA | 8 | <0.1 | <0.1 | 2 | 9 | 3 |
| 58 | | | | | | | |
| SEQ ID NO: | BD-FBPA- | 15 | <0.1 | <0.1 | 2 | 17 | 2 |
| 50 | BYDV | | | | | | |
| | CORE PRO | | | | | | |
| SEQ ID NO: | BD-FBPA- | 12 | <0.1 | <0.1 | <0.1 | 18 | 1 |
| 51 | HLCV | | | | | | |
| | CORE PRO | | | | | | |
| SEQ ID NO: | BD-FBPA- | 38 | <0.1 | <0.1 | 4 | 65 | 10 |
| 52 | BSV(AY) | | | | | | |
| | CORE PRO | | | | | | |

A set of hybrid regulatory elements was made consisting of non-redundant parental segments. The segment providing the TATA box (SEQ ID NO: 48) generates expression in leaf and silk tissues, but where silk expression is lower than the leaf (Table 8). In an effort to increase the silk expression, segments from two different from silk-preferred promoters (SEQ ID NO: 61 and 62) were tested in combination with SEQ ID NO: 48. In the first hybrid regulatory element, one segment from SEQ ID NO: 61 was added (SEQ ID NO: 47). An increase in silk expression was observed. Leaf and root expression remained about the same, and stalk expression increased slightly. Adding SEQ ID NO: 62 (SEQ ID NO: 56) resulted in a more significant increase in silk expression. Leaf and stalk expression decreased slightly, and root expression had no change. In a third hybrid regulatory element, the 5′ most silk segment from the hybrid regulatory element of SEQ ID NO: 56 was replaced with a green tissue segment (SEQ ID NO: 64) to produce the aerial hybrid regulatory element of SEQ ID NO: 57. The result was an increase in leaf expression and a return of silk expression to previous levels. Stalk expression increased slightly and root expression remained unchanged. These results demonstrate the capability to use non-redundant parent regulatory element segments, where the non-redundant segments may be from a different species (in this case Brachypodium distachyon), to produce a hybrid regulatory element that functions in targeted tissues of maize

TABLE 8
Expression Results for Each Hybrid Regulatory
Element in Transgenic Maize

| Upstream | | | | Root | | | |
|---|---|---|---|---|---|---|---|
| Activation | | | Root | (mature | | | |
| Element | Base | Leaf | (tip) | region) | Stalk | Silk | Husk |
| SEQ ID NO: | ZM-SILK12 | 31 | <0.1 | <0.1 | 3 | 10 | 16 |
| 48 | PRO | | | | | | |
| SEQ ID NO: | ZM-SILK12 | 30 | <0.1 | <0.1 | 8 | 18 | 18 |
| 47 | PRO | | | | | | |
| SEQ ID NO: | ZM-SILK12 | 23 | <0.1 | <0.1 | 4 | 36 | 20 |
| 56 | PRO | | | | | | |
| SEQ ID NO: | ZM-SILK12 | 43 | <0.1 | <0.1 | 7 | 10 | 20 |
| 57 | PRO | | | | | | |

Similar to root hybrid regulatory elements, the segments relative orientation to each other may impact the expression result in hybrid regulatory elements designed for expression in other tissues (see FIG. 2 for segment orientation). Table 9 shows expression results a green tissue segment is placed at the 5′ end of a hybrid regulatory element (SEQ ID NO: 54) versus a 3′ position where it is providing the TATA box (SEQ ID NO: 55). When the green tissue segment is placed in the 5′ position, there is no increase in leaf expression (SEQ ID NO: 54; Table 9). However, when the green tissue segment is placed in the 3′ position, leaf expression increases. Position also affected husk expression with somewhat of a reverse effect.

TABLE 9
Expression of Relative Orientation of Segments of a
Hybrid Regulatory Element in Transgenic Maize

| Upstream | | | | Root | | | |
|---|---|---|---|---|---|---|---|
| Activation | | | Root | (mature | | | |
| Element | Base | Leaf | (tip) | region) | Stalk | Silk | Husk |
| SEQ ID NO: | ZM-SILK12 | 31 | <0.1 | <0.1 | 3 | 10 | 16 |
| 48 | PRO | | | | | | |
| SEQ ID NO: | ZM-SILK12 | 30 | <0.1 | <0.1 | 8 | 18 | 18 |
| 47 | PRO | | | | | | |
| SEQ ID NO: | ZM-SILK12 | 30 | <0.1 | <0.1 | 5 | 40 | 23 |
| 54 | PRO | | | | | | |
| SEQ ID NO: | ZM-SILK12 | 40 | <0.1 | <0.1 | 4 | 33 | 12 |
| 55 | PRO | | | | | | |

In another example, two copies of the silk segment used in Table 7 (from the parental regulatory element of SEQ ID NO: 60) were placed upstream of two copies of the green tissue segment also in Table 7 (from the parental regulatory element of SEQ ID NO: 49). Leaf and silk expression increased about two-fold relative to the parents in this configuration (SEQ ID NO: 53; Table 10). However, reversing the arrangement so that two copies of the green tissue segment are upstream of the two copies of the silk segment resulted in no leaf expression (SEQ ID NO: 59; Table 10). The combined results suggest the segment providing the TATA box or 3′ segments may impact or influence expression provided by the hybrid regulatory element.

TABLE 10
Expression of Relative Orientation of Segments of a
Hybrid Regulatory Element in Transgenic Maize

| Upstream | | | | Root | | | |
|---|---|---|---|---|---|---|---|
| Activation | | | Root | (mature | | | |
| Element | Base | Leaf | (tip) | region) | Stalk | Silk | Husk |
| SEQ ID NO: | BD-FBPA | 10 | <0.1 | <0.1 | 1 | 1 | 3 |
| 49 | PRO | | | | | | |
| SEQ ID NO: | ZM-OEBF | <0.1 | <0.1 | <0.1 | <0.1 | 5 | <0.1 |
| 60 | PRO | | | | | | |
| SEQ ID NO: | BD-FBPA | 20 | <0.1 | <0.1 | 2 | 10 | 6 |
| 53 | PRO | | | | | | |
| SEQ ID NO: | ZM-OEBF | <0.1 | <0.1 | <0.1 | <0.1 | 6 | <0.1 |
| 59 | PRO | | | | | | |

### Example 10: Agrobacterium-Mediated Transformation of Maize and Regeneration of Transgenic Plants

For Agrobacterium-mediated transformation of maize polynucleotides of the disclosure, the method of Zhao was employed (U.S. Pat. No. 5,981,840, and PCT patent publication WO98/32326). Briefly, immature embryos were isolated from maize and the embryos contacted with a suspension of Agrobacterium under conditions whereby the bacteria were capable of transferring the regulatory element sequence of the disclosure to at least one cell of at least one of the immature embryos (step 1: the infection step). In this step the immature embryos were immersed in an Agrobacterium suspension for the initiation of inoculation. The embryos were co-cultured for a time with the Agrobacterium (step 2: the co-cultivation step). The immature embryos were cultured on solid medium following the infection step. Following the co-cultivation period an optional “resting” step was performed. In this resting step, the embryos were incubated in the presence of at least one antibiotic known to inhibit the growth of Agrobacterium without the addition of a selective agent for plant transformants (step 3: resting step). Next, inoculated embryos were cultured on medium containing a selective agent and growing transformed calli were recovered (step 4: the selection step). Plantlets were regenerated from the calli (step 5: the regeneration step) prior to transfer to the greenhouse.

1. A hybrid regulatory element comprising an assembly of heterologous regulatory element segments, wherein the assembly of the heterologous regulatory element segments produces a unique tissue expression pattern.

2. (canceled)

3. (canceled)

4. The hybrid regulatory element of claim 1, wherein the heterologous assembly of regulatory element segments comprises the assembly of at least three parent regulatory element segments.

5. The hybrid regulatory element of claim 1, wherein the heterologous assembly of regulatory element segments comprises the assembly of at least four parent regulatory element segments.

6. The hybrid regulatory element of claim 1, wherein the tissue expression pattern is a root expression pattern or an aerial expression pattern.

7. The hybrid regulatory element of claim 1, wherein the tissue expression pattern is a ubiquitous aerial expression pattern.

8. The hybrid regulatory element of claim 1, wherein the hybrid regulatory element produces a ubiquitous root expression pattern and each parent regulatory element produces regional or sub-regional root or aerial tissue expression.

9. The hybrid regulatory element of claim 1, wherein the heterologous assembly of regulatory element segments comprises more than one copy of the same segment from a single parent regulatory element.

10. The hybrid promoter of claim 1, wherein the heterologous assembly of regulatory element segments comprises parent regulatory element segments from more than one species.

11. The hybrid promoter of claim 1, wherein the heterologous assembly of regulatory element segments comprises parent regulatory element segments from at least one virus derived parent regulatory element or plant derived parent regulatory element.

12. A DNA construct comprising the hybrid regulatory element of claim 1.

13. The DNA construct of claim 12, wherein the hybrid regulatory element is operably linked to a polynucleotide of interest.

14. An expression cassette comprising the DNA construct of claim 13.

15. A host cell comprising the expression cassette of claim 14.

16. The host cell of claim 14, wherein the host cell comprises a plant cell, a bacterial cell, or an animal cell.

17. A transgenic plant comprising the expression cassette of claims 14.

18-24. (canceled)

25. A method of creating a hybrid regulatory element comprising:
a. Identifying a native regulatory element with a regional expression pattern;
b. Identifying motifs of the native regulatory element; and
c. Assembling at least two segments into a hybrid regulatory element that initiates transcription in a plant cell.

26. The method of claim 25, further comprising expressing a heterologous polynucleotide of interest operably linked to the hybrid regulatory element.

27. The method of claim 26, wherein the heterologous polynucleotide sequence of interest comprises a gene product that confers herbicide, salt, cold, drought, pathogen, or insect resistance.

28. The method of claim 26, wherein the heterologous polynucleotide sequence of interest is expressed in a root expression pattern or an aerial expression pattern.

29. (canceled)

30. (canceled)

31. A method for directed expression of a polynucleotide sequence in a plant or plant cell, said method comprising introducing into the plant or the plant cell an expression cassette comprising a hybrid regulatory element operably linked to a heterologous polynucleotide sequence of interest, wherein said hybrid regulatory element produces a unique tissue expression pattern of the heterologous polynucleotide sequence of interest.

32. The method of claim 31, wherein the heterologous polynucleotide sequence of interest is expressed in a root expression pattern or an aerial expression pattern.

33. (canceled)

34. (canceled)

35. The method of claim 31, wherein the hybrid regulatory element comprises an assembly of at least three parent regulatory element segments.

36. The method of claim 31, wherein the hybrid regulatory element comprises an assembly of at least four parent regulatory element segments.

37. The method of claim 31, wherein the tissue expression pattern is a root expression pattern or an aerial expression pattern.

38. The method of claim 37, wherein the hybrid regulatory element produces a ubiquitous root expression pattern and the parent regulatory elements produce regional or sub-regional root or aerial tissue expression.

39. The method of claim 31, wherein the hybrid regulatory element comprises more than one copy of a segment from a single parent regulatory element.

40. The method of claim 31, wherein the hybrid regulatory element comprises parent regulatory element segments from more than one species.


SEQUENCE LISTING (74 sequences)

SEQ ID NO: 1 - DNA, 962 bp, Brachypodium distachyon
  misc_feature 1..962: note = BD-RCI2 PRO (MOD 1)
  source 1..962: mol_type = other DNA
tttgacttggaccgactagcactcccccgctaggcacgtagtacgtggtatgtgtggaaacgattggttcgttgtagggagtcagtcagatcagatatgatgcttctcgtgtgttgattttcttcctctgaactgaaaggaagcatgcaagttttacaagctaaactcagcagcggaagctatgcatacgttccactggctgcagaacagggcaggccggcggctagctcctgtgagctgtgttggaggagaataatatcttgtcgtttggctttgctcgggtggatacttttttttcttttgacagctgctcgggtggatactgatacactatgaagaccgaaaaaaagaagcgaagcatataaacagacggatcattgggggccaagccagcaggtagtgcggtggaacattatgtagaatgaatcggttcttgctgtgcagacagctcagctcagcacactcctggcccaaaacaaagtgcatgtggtgtattttcgtatactagctaacaaatgcttaagcccctgtttgaaatattgtattttcggcggacgggcactggcttactgaaatcttgccaggaggcacggacaggtgcttagcagccaagtaatgtacagtaaatccggcaaattgccaggggagtggttcgattcaatcaacgaatgcgattcccgatataaattatccccagtttatttttgattttatacaacgcgtcgccgacgcggtccccaccccgtgcgcgtcgcaaggtccacacgtgtcctctctccgacgcttcctagctcacgccgtccttgattgacctgcctcgccatctataaatggcaaaccaggctaaggctcatttctatactccctcttcctgctctgctctctgctctagtttactccactgacccaaagagagaaaagaaagcactcagaacttgagatcagcatccaggagatcggcc

SEQ ID NO: 2 - DNA, 1792 bp, Brachypodium distachyon
  misc_feature 1..1792: note = BD-GRP3 PRO (MOD 1)
  source 1..1792: mol_type = other DNA
tctggcgtagtgtggatagtttacgtcaaattctattgcgtcggtagtagatttttgcaaaacatcagcttaacatttcacaagtttaagtgtttaaccactgagtgactttttaatcgtttagttctaacttaagaacatgatgttttgtagatggagaacatgtgcattccagaagtcagaactagaagcgatcgaaatgaactgatctgaagagatcgagctgtgttagtactatattattgttgattctagaccattaaagatcggatcgcgtcaactaatatggcaactagctaggatctgttaccttaattctcgtttctatcgatctatttggggctcttctacacatgagctagctggagaatcgaataccaagtcctaacctgcaacttggacactgcaattttcatcgatcgacatctctcgttgtgtgcgttgccataactattcgaagatagactaataactgatacagtgcgttcctgtctgtacggtcaaacttctggacccctggcaatgcgtaaaaccctaggtcaccacacggtacggtgtgacacagctaagcgcaattaatacccaagtcaacactctgcgtcgtcgtttaccaacagattaattagctcaactcgctcaactcgccgagtagctagcagtgaaaagggaaacgcgcggcactacattactgcggcgcttgctcaaacttcatgcaccaataattgaaacacggtcaattgtatgcattactagtgtaattaagtgcatgtaacgaatgaactaccttaggattttaggagaatcgaagaatatctccatccatttaccaaatcaaacttattaagaagaaaatattatgtacttcctacgtccaaaaaagatgtctcaagtttgtcaaattttggatgtatctagacatgacttaatgtatagatgcattcaaattttgacaaacttaagacatccttttttggacggagggagtacaagatttgttaataagttacattaaaaaactacacagtactgtctactcgtttttttttctaaagaaaatcacggatctattaataatcaccaacatgagccttacttaattaagtacttagagacgacgtcgagcgtttggagagtctttgtacaatgtcaaaggtacctataatccggccggccgggtgatttgaatcgcgcgcctagctagatcgatccatgcatgcgatccacagctagcttgatgcttgcgccagtgtgtgatcgatcatgagctcatgaccccatgacatcgagcgagttagctagagaagccatgcacgcacgttctttcacgtggaagagttataaaaagctagcgtaatacgtttatatatgtcaggagatgggaatctctagctaattaggcttcgagagaaaccaagcaacagctagtaattaacttcagatccttccgcactcgataatcaagtcttgatctgcatagcccagtgaagtgccaataaatctaagatgtgaagagatgtgcaacagtgcaagagaaaatttaatgctgtctagttgcttgatcgatgcagtcaatactagtactgtcaccctgattagaaaaattatcactcatcgaatcacccacttgataattcacagcccgaagtcgtcccgaagcgcgcggtcgcctccttgagatatccaaccccatgcatctcatctaccactataaatactcaggccaccctctctgctaagcaccccaccacctcatctcacacgtactctagctacaaattaagctacgtcggca

SEQ ID NO: 3 - DNA, 2001 bp, Brachypodium distachyon
  misc_feature 1..2001: note = BD-RCC3B PRO (MOD 1)
  source 1..2001: mol_type = other DNA
aaaactaggttcaagacacatttcaactcctcccttcatcccattttaagtgctgaaatattacatgtatctaaacgtcttttaggtatacatacattcagatgttcattaaatgacagggtgctgacgtggcatacacgctatgtgtatcagatgaaaaacatagctagcaaacacatctggctttcatacgtacatcagcagatgcaaggtcgacgaatacatgcattcgtcaacctctagcggctggtagcaacatcctcacaacgaaaacgctagccggaagccaacgtaccataggcgcacacaaatccacgaccacgactatgaaccggatacctgaagtcaagcatctctcggtaccggccgccgaaagcaacaacaacaccagaaacaacctgacaaaataatgaagatttcgtgtctaacgttaaaaagaaattatgaaagagtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgt

SEQ ID NO: 4 - DNA, 1843 bp, Zea mays
  misc_feature 1..1843: note = ZM-PCO118362A PRO (FL)
  misc_feature 1..1843: note = ZM-PCO118362A PRO (TR 1)
  source 1..1843: mol_type = other DNA
agcaagatcggtgaacaacactgagcatgggcttttgaggcttttgactgatgacagcacagataaaattgaggatagtacatcagttttagttgcagcactaaaacaatgctagtctactaatggctttcagccaagagaactgctggtgctcagtttagtttctgcaaggctatcctttccaacatcagcaagcttttccctacttaatgcagagggcatgctttgtatatcttcactaattttgttttattaggatgccgcaaaggccaaagggttgatctttcatccctccatttattttgcccacttgcatttgaaaaaaagaaaagggtgtcgatgtttagaacggaggtagtaataactagctgcttaatctagttgtcaccatggatggagaaatcagataaacctgcattagcactaagcacaaatgtgtatgtatggttatgtaatggtagcatggagcatgattttcatttttcatatgaacatagagcatatataataatatgtgtaagcaccaaggggtaagatgtgttctaaccttactcaaatactaggcttaaatctagagcaagcactatataagtagaagtctatctaacctaggcactttataaaacacctacgctaattaggagtgattcttttgaacaattgggtatctggagctactagagataagcgttaactttcttaatattttccttaattacctcccatacatcttaaatgtctagtggtagcatatatacctccaaagtccaaagctagttgtaagtgacacaaaaaattatgcgtactcattgaacatatctgttacactcactaactagacatgtttagtgcatctacgttagctagttgcagaccccaatcctatattgttcataaatttttatcataaggttccctgctgcaatttagatatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 5 - DNA, 1397 bp, Sorghum bicolor
  misc_feature 1..1397: note = SB-PLTP PRO
  source 1..1397: mol_type = other DNA
caaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatggcgtcgctccatctcgcatctataaataccattggccatgcacacccgcactcccacacagcaatacagcacactagcagcagcagcagcagctcgagctagcttagctactacgtgtgtgcaatcagctcgatcgcc

SEQ ID NO: 6 - DNA, 1710 bp, Sorghum bicolor
  misc_feature 1..1710: note = SB-RCC3 PRO (MOD 1) [RCST fix]
  source 1..1710: mol_type = other DNA
ttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccgggtctcaactataaatacccccttggtgacaccgcgatcaaagcatcgcaaacaagcctagctaagagctctctaactacattagatagagtgatctcgagaggtaactggcttgtgatcgagca

SEQ ID NO: 7 - DNA, 1745 bp, Sorghum bicolor
  misc_feature 1..1745: note = SB-TIP2-3 PRO (FL)
  source 1..1745: mol_type = other DNA
gcgcagttaagcgcacttgtggattggactgttgccaatatcattgtgataataatcagaaccagttgcatgactcctctccatactgcaaaattacattctactaatgtatcatcatccttttcagtttatttgtcactaccaaactactatggcagtttttttttaataaacgtaaaaatgtggcaaattttaccgctcgttttcctcaggctatgcatttggttttaaccaacaattcggttcgatttatacgcttcatagaaatttcggttcccagaaactaaaaaccgatcagttattcatgaaattaaaagtgccatgttttccccctaaagtttatatatacctgaaagtgtacaatgctaagtatgtttcactataaatcaattcatgaggaattttcaagcatttataatctttcccaaaaaaaaggtgcgcaatcaaaatttcttacaaaacattccagtgacagatcatcagacaatagtgtttctgataaaacccccacgtaggatcagagaaagtttagagaatattcccacagtggcccaaaacattttgtctgacacgcagggagcgcataagttttttttattttattattgaaaagtgagttctaggaaactctatacacacataggctgaacaaccgtaaggatagcataaagaaataaggcctttttggtgtcatcatcttcgtttgtgcaataatactgcaactcaatgaagtcactgcgactcataagagccctgtcatcaccttgcaactggcagttttcaattggtgtaccagcgcaagatccatagattaatcttgtttgcctcacctaccccaaatggaggatgttcactgcattgatgtcacaatccgaccttcttaaccgctctatgtcacaactttaacatgaatgatatatcgtttcctcaaaaacatgaatgatatatcactacagaactcagtatgcagtagattcctgaacgcaagagctgttgcaatgttaatctactaggtgatatcttactcggtaagatactcaggccgaggtccgaggaattacccttttgttgcaggagaaccaagtcatatgaatgaagcagcagctcagtaatgatccaccgactacgctacttctagcatagaattagtaatcttttctaatactgttgtaaatttgtaacaacaaaacaagaaccacgccacggaagaacggtgagaaaaagaaacctttgctaacgtactattcatgaatttgaagattttgtatggatgcatcagtagacgatgcagggtagaaataaaggatcgattcagataatcaatccgcaccgccattgatttgtttagggatgagatgagaattgagaattgggataacatatatctgctccaaggtcttgttctcctcgctgtcttatggtgtatcctaacgtggctcggcctaaacaaccacaattccacagcagtttagcgagttagggcggttggtcggccaatatcagccacaaaacacgaagcaaaatttaaattatcaatcatgtggtgatcattgcacaccgcccatagtattgtattgcacatctgaggcagggctccagcctccaggtccattagcgccttatgatgtgtatttaaggagactgagctgaaggaactctcgcatcagcctgataagctatagccagccatcttctctgaattccaactcagtccaagggctggaagctagaagtaccgtcagaga

SEQ ID NO: 8 - DNA, 1139 bp, Zea mays
  misc_feature 1..1139: note = ZM-CYCLO1 PRO
  source 1..1139: mol_type = other DNA
ctcctcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacttataataattatccgtatagtctagtacgtacgggacatcacgccattgcctgttgtgtataaaaagcgagcatgagctaggttgggagcagagcaaagcgtagtcatcacctgtgtctaggttgggagcaaagcaaagagagagaggaaagctagctagctagc

SEQ ID NO: 9 - DNA, 1343 bp, Zea mays
  misc_feature 1..1343: note = ZM-ROOTMET2 PRO
  source 1..1343: mol_type = other DNA
tttttactaaattcatgtaataattaatgtatgcgttatatatatatgtctaggtttataattattcatatgaatatgaacataaaaatctagggctaaaacgactactattttgaaaacggaaggagtagtaagttatttaagcggaggggaaccatgatgggctagtgatttaatttacatatatatattggtgttctgggctcttacatgagaagatctagttaactgttgttactgaacagcgaagacaaatatataatttaagctccccaactgctagtgattctgttaagaggtaatgtttaaagtaaatttacaagagcccgtctagctcagtcggtagagcgcaaggctcttaaccttgtggtcgtgggttcgagccccacggtgggcgcacaattttttgttttttgacattttttgtttgyttagttgcagacggtttttcccctgctaggagatttccgagagaaaaaaaaggcactacaggttaaccaaaaccaccaacctttggagcgtcgaggtcgacgggcatttgcgtagttgaagcttacaaagttgcatatgagatgagtgccggacatgaagcggataacgttttaaactggcaacaatatctagctgtttcaaattcaggcgtgggaagctacgcctacgcgccctggacggcgtgtaaagagccagcatcggcatcattgtcaaacgatcgacaaggccaagaaattccaaatatattattaataaaaaagaaggcacaaattagtttggttttttagtatgtgtggcggaggaaattttgagaacgaacgtatcaaagaaggcacaagacgatatagattgacgcggctagaagttgcagcaagacagtgggtacggtcttatatatcctaataaataaaaaataaaactatagtgtgtcaaatgtcaacaagaggaggaggcagccaaattagcagagggagacaagtagagcacgccttattagcttgcttatttatcgtggtggtgtacttgttaattactggcacgcattatcaacaacgcagttctggatgtgaatctagacaaacatttgtctaggttccgcacgtatagtttttttcctcttttttttggggggggggtgggggggggaacggaagctgtaataaacggtactaggaacgaaagcaaccgccgcgcgcatgtttttgcaatagattacggtgaccttgatgcaccaccgcgtgctataaaaaccagtgtccccgagtctactcatcaaccaatccataactcgaaaccttttcttgtgctctgttctgtctgtgtgtttccaaagcaagcgaaagaggtcgagg

SEQ ID NO: 10 - DNA, 2078 bp, Setaria italica
  misc_feature 1..2078: note = SI-TIP2-3B (MOD 1)
  source 1..2078: mol_type = other DNA
aaagctcctaccattttatttgcaatagtagatgcatataattgatggacacagagttctgaaccgtgagattctctttaacttcagagttctgtcactattttaacgtggatctggagttttgcaggaactgtgaagaaggaaccgtacctaaaactaaaagtctcacgaagaccaaatgggcaagccagacagtcagagagacagggcttcttcggccaactggacaagggatgtgcctacatgtgctgagagattaccaaatccgcaatttgcagacgtgccttctcaatcagtttgcacaaagcgataaggtaaacaaccattggagcattgttaacagcagcctagcatggcaggatgcgtcatctgaataatgcggagggcaggacagtaggacaggacacctaatccctgcaaattctagtggctctgggacactgatcaaggcccaaaagtgctaaccactgcattactaaaacgtgtgggtgttgcgagttccaagttggctgttgtcatggaatggtatgaaaatgcaaaagattctcgctgttttgttgagagttagtaaattgggatgacttcggcatgacaacagcacaaaccttacatatttggtaagacttgcaacaaaaatcaaaagaatgctagtagataccaacaaaaaggaaaaacagagctagcatgaacacttctttgatcacagttgatcaaaggtgtatggccctcagacaaagctgttgatgatggtcaaacacttttgctaccgaatatcgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcgagagggcgacgccatgtgtataaaaggggcctaagctgagtggacttgctgcatcagattagtaagcaatctcaagcgcagagagccaaagctttcggtgtagctcgaagagcaaagcgaaggcaaggtcgt

SEQ ID NO: 11 - DNA, 4112 bp, synthetic construct
  misc_feature 1..4112: note = synthetic
  misc_feature 1..4112: note = 3X CYCLO-PCOa
  source 1..4112: mol_type = other DNA
ctcctcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctcgacctcctcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctcgacctcctcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacttataataattatccgtatagtctagtacaactttcttaatattttccttaattacctcccatacatcttaaatgtctagtggtagcatatatacctccaaagtccaaagctagttgtaagtgacacaaaaaattatgcgtactcattgaacatatctgttacactcactaactagacatgtttagtgcatctacgttagctagttgcagaccccaatcctatattgttcataaatttttatcataaggttccctgctgcaatttagatatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcgg

SEQ ID NO: 12 - DNA, 5676 bp, synthetic construct
  misc_feature 1..5676: note = synthetic
  misc_feature 1..5676: note = 3X RCC3-PCOa(TR2)
  source 1..5676: mol_type = other DNA
ttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatttcgaattttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatgtcgacgggccgcttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgttcgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 13 - DNA, 2789 bp, synthetic construct
  misc_feature 1..2789: note = Synthetic
  misc_feature 1..2789: note = Root Hybrid 2
  source 1..2789: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatccccgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 14 - DNA, 3137 bp, synthetic construct
  misc_feature 1..3137: note = Synthetic
  misc_feature 1..3137: note = Root Hybrid 13
  source 1..3137: mol_type = other DNA
ttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgggcactacattactgcggcgcttgctcaaacttcatgcaccaataattgaaacacggtcaattgtatgcattactagtgtaattaagtgcatgtaacgaatgaactaccttaggattttaggagaatcgaagaatatctccatccatttaccaaatcaaacttattaagaagaaaatattatgtacttcctacgtccaaaaaagatgtctcaagtttgtcaaattttggatgtatctagacatgacttaatgtatagatgcattcaaattttgacaaacttaagacatccttttttggacggagggagtacaagatttgttaataagttacattaaaaaactacacagtactgtctactcgtttttttttctaaagaaaatcacggatctattaataatcaccaacatgagccttacttaattaagtacttagagacgacgtcgagcgtttggagagtctttgtacaatgtcaaaggtacctataatccggccggccgggtgatttgaatcgcgcgcctagctagatcgatccatgcatgcgatccacagctagcttgatgcttgcgccagtgtgtgatcgatcatgagctcatgaccccatgacatcgagcgagttagctagagaagccatgcacgcacgttctttcacgtggaagagttataaaaagctagcgtaatacgtttatatatgtcaggagatgggaatctctagctaattaggcttcgagagaaaccaagcaacagctagtaattaacttcagatccttccgcactcgataatcaagtcttgatctgcatagcccagtgaagtgccaataaatctaagatgtgaagagatgtgcaacagtgcaagagaaaatttaatgctgtctagttgcttgatcgatgcagtcaatactagtactgtcaccctgattagaaaaattatcactcatcgaatcacccacttgataattcacagcccgaagtcgtcccgaagcgcgcggtcgcctccttgagatatccaaccccatgcatctcatctaccactataaatactcaggccaccctctctgctaagcaccccaccacctcatctcacacgtactctagctacaaattaagctacgtcggca

SEQ ID NO: 15 - DNA, 2337 bp, synthetic construct
  misc_feature 1..2337: note = Synthetic
  misc_feature 1..2337: note = Root Hybrid 17
  source 1..2337: mol_type = other DNA
ttagttgcagacggtttttcccctgctaggagatttccgagagaaaaaaaaggcactacaggttaaccaaaaccaccaacctttggagcgtcgaggtcgacgggcatttgcgtagttgaagcttacaaagttgcatatgagatgagtgccggacatgaagcggataacgttttaaactggcaacaatatctagctgtttcaaattcaggcgtgggaagctacgcctacgcgccctggacggcgtgtaaagagccagcatcggcatcattgtcaaacgatcgacaaggccaagaaattccaaatatattattaataaaaaagaaggcacaaattagtttggttttttagtatgtgtggcggaggaaattttgagaacgaacgtatcaaagaaggcacaagacgatatagattgacgcggctagaagttgcagcaagacagtgggtacggtcttatatatcctaataaataaaaaataaaactatagtgtgtcaaatgtcaacaagaggaggaggcagccaaattagcagagggagacaagtagagcacgccttattagcttgcttatttatcgtggtggtgtacttgttaattactggcacgcattatcaacaacgcagttctggatgtgaatctagacaaacatttgtctaggttccgcacgtatagtttttttcctcttttttttggggggggggtgggggggggaacggaagctgtaataaacggtactaggaacgaaagcaaccgccgcgcgcatgtttttgcaatagattacggtgaatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgggcactacattactgcggcgcttgctcaaacttcatgcaccaataattgaaacacggtcaattgtatgcattactagtgtaattaagtgcatgtaacgaatgaactaccttaggattttaggagaatcgaagaatatctccatccatttaccaaatcaaacttattaagaagaaaatattatgtacttcctacgtccaaaaaagatgtctcaagtttgtcaaattttggatgtatctagacatgacttaatgtatagatgcattcaaattttgacaaacttaagacatccttttttggacggagggagtacaagatttgttaataagttacattaaaaaactacacagtactgtctactcgtttttttttctaaagaaaatcacggatctattaataatcaccaacatgagccttacttaattaagtacttagagacgacgtcgagcgtttggagagtctttgtacaatgtcaaaggtacctataatccggccggccgggtgatttgaatcgcgcgcctagctagatcgatccatgcatgcgatccacagctagcttgatgcttgcgccagtgtgtgatcgatcatgagctcatgaccccatgacatcgagcgagttagctagagaagccatgcacgcacgttctttcacgtggaagagttataaaaagctagcgtaatacgtttatatatgtcaggagatgggaatctctagctaattaggcttcgagagaaaccaagcaacagctagtaattaacttcagatccttccgcactcgataatcaagtcttgatctgcatagcccagtgaagtgccaataaatctaagatgtgaagagatgtgcaacagtgcaagagaaaatttaatgctgtctagttgcttgatcgatgcagtcaatactagtactgtcaccctgattagaaaaattatcactcatcgaatcacccacttgataattcacagcccgaagtcgtcccgaagcgcgcggtcgcctccttgagatatccaaccccatgcatctcatctaccactataaatactcaggccaccctctctgctaagcaccccaccacctcatctcacacgtactctagctacaaattaagctacgtcggca

SEQ ID NO: 16 - DNA, 2984 bp, synthetic construct
  misc_feature 1..2984: note = Synthetic
  misc_feature 1..2984: note = Root Hybrid 14
  source 1..2984: mol_type = other DNA
ttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgcccgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatggtttgacttggaccgactagcactcccccgctaggcacgtagtacgtggtatgtgtggaaacgattggttcgttgtagggagtcagtcagatcagatatgatgcttctcgtgtgttgattttcttcctctgaactgaaaggaagcatgcaagttttacaagctaaactcagcagcggaagctatgcatacgttccactggctgcagaacagggcaggccggcggctagctcctgtgagctgtgttggaggagaataatatcttgtcgtttggctttgctcgggtggatacttttttttcttttgacagctgctcgggtggatactgatacactatgaagaccgaaaaaaagaagcgaagcatataaacagacggatcattgggggccaagccagcaggtagtgcggtggaacattatgtagaatgaatcggttcttgctgtgcagacagctcagctcagcacactcctggcccaaaacaaagtgcatgtggtgtattttcgtatactagctaacaaatgcttaagcccctgtttgaaatattgtattttcggcggacgggcactggcttactgaaatcttgccaggaggcacggacaggtgcttagcagccaagtaatgtacagtaaatccggcaaattgccaggggagtggttcgattcaatcaacgaatgcgattcccgatataaattatccccagtttatttttgattttatacaacgcgtcgccgacgcggtccccaccccgtgcgcgtcgcaaggtccacacgtgtcctctctccgacgcttcctagctcacgccgtccttgattgacctgcctcgccatctataaatggcaaaccaggctaaggctcatttctatactccctcttcctgctctgctctctgctctagtttactccactgacccaaagagagaaaagaaagcactcagaacttgagatcagcatccaggagatcggcc

SEQ ID NO: 17 - DNA, 3982 bp, synthetic construct
  misc_feature 1..3982: note = Synthetic
  misc_feature 1..3982: note = Root Hybrid 4
  source 1..3982: mol_type = other DNA
tacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcaaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgagtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgt

SEQ ID NO: 18 - DNA, 3539 bp, synthetic construct
  misc_feature 1..3539: note = Synthetic
  misc_feature 1..3539: note = Root Hybrid 5
  source 1..3539: mol_type = other DNA
tacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcctttttggtgtcatcatcttcgtttgtgcaataatactgcaactcaatgaagtcactgcgactcataagagccctgtcatcaccttgcaactggcagttttcaattggtgtaccagcgcaagatccatagattaatcttgtttgcctcacctaccccaaatggaggatgttcactgcattgatgtcacaatccgaccttcttaaccgctctatgtcacaactttaacatgaatgatatatcgtttcctcaaaaacatgaatgatatatcactacagaactcagtatgcagtagattcctgaacgcaagagctgttgcaatgttaatctactaggtgatatcttactcggtaagatactcaggccgaggtccgaggaattacccttttgttgcaggagaaccaagtcatatgaatgaagcagcagctcagtaatgatccaccgactacgctacttctagcatagaattagtaatcttttctaatactgttgtaaatttgtaacaacaaaacaagaaccacgccacggaagaacggtgagaaaaagaaacctttgctaacgtactattcatgaatttgaagattttgtatggatgcatcagtagacgatgcagggtagaaataaaggatcgattcagataatcaatccgcaccgccattgatttgtttagggatgagatgagaattgagaattgggataacatatatctgctccaaggtcttgttctcctcgctgtcttatggtgtatcctaacgtggctcggcctaaacaaccacaattccacagcagtttagcgagttagggcggttggtcggccagtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgt

SEQ ID NO: 19 - DNA, 3789 bp, synthetic construct
  misc_feature 1..3789: note = Synthetic
  misc_feature 1..3789: note = Root Hybrid 1 Pro
  source 1..3789: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccctaggtcaactataatactgtactccctccgtcctcgaaagcttcaactcttagaatccgtgccagtcaaacttttctaactttgaccaactttataagagtaagtttaataatacagcccacttgctggctgtaaggtttcttacagccttcttccagcccacccatacaatagttagctattcactattaatacatggcccacttgtctctctcacaaagttccttggttcctgtccctaagctggctgtaagcttacagcccgcttctcctctctctcctctcttctctcctccacctcagcatttagccagcttacagcccactataatacttgctctaaaaaagagtatcaatatctatgacataaaataagtatcttacgaaaatatattctataataaatttaatggtattaatttggtactataaatcttagcatttttttctataaatttagtcaaagttgtaaaagtttgacttaagacaactctaaaaattgcagcgggagtatgtatgatcgatgctgctctagtttttccagcccacctctcgttgtttgcgacttcgagtttactgcatctatatctctgaaagaaaatgcctacagggcttggccggtgtcagcttcagggctctctgtaggcgttgaatccgaggccgccgtagggtggtggcagcgcgaccatcgagccgtggagcaagtacgagtcaaacacgctgagccgcgtaaccgttttaataaaatgaactctactcgatcgctaggtcacagcagcacgatcacagtgaagaatgcgtgaggggctcgagcgttggcgttgttgctgtgctttgatcaaactgcaccggcgagaaggaagtaatataagcaactcgatcataatccaagcagtaaacgcctgatcaattgtcgacatctggctggcactagtattactagattgtagaatacgccgcgtgatgtgatttgttattttaggtccaaattcatgaagaattgtggagtagttgagtagatgagtacagtgacttgcagttgcagcaaacgattgatgttcacatgacagggttcaggggtcctgtagtcctgttacttgcagtattgccgtatcgtgaatctgaagtcgaacagagatagatcgatggcgtaatgtcccaaggaggagagaaataatatgactgtatcactgtagtgtgtagtcgatgggaatctatagctctcgttattaggctcttaattttaacagaaaccaaagcaactacaagctaattaattccagagacttctacagtgccacatcattcaattggcaccagaattaaaatagtttttcaagaaatcgcatctcagaaaaagatgctaatggtttctttatcgttgtggtgaacactagtgccatccagattacagaattctctcctgatcggatctcatcgcacatggacagccctgcaaacccaacacggaaaccctgaaagatcgcgtccatgcacgaccccccacagctataaatacccatgcaacgcagcaggtcatcatccacctcgcctcattctcactgcatcaccgagctcactggcta

SEQ ID NO: 20 - DNA, 4280 bp, synthetic construct
  misc_feature 1..4280: note = Synthetic
  misc_feature 1..4280: note = Root Hybrid 3 PRO
  source 1..4280: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccctaggtcaactataatactgtactccctccgtcctcgaaagcttcaactcttagaatccgtgccagtcaaacttttctaactttgaccaactttataagagtaagtttaataatacagcccacttgctggctgtaaggtttcttacagccttcttccagcccacccatacaatagttagctattcactattaatacatggcccacttgtctctctcacaaagttccttggttcctgtccctaagctggctgtaagcttacagcccgcttctcctctctctcctctcttctctcctccacctcagcatttagccagcttacagcccactataatacttgctctaaaaaagagtatcaatatctatgacataaaataagtatcttacgaaaatatattctataataaatttaatggtattaatttggtactataaatcttagcatttttttctataaatttagtcaaagttgtaaaagtttgacttaagacaactctaaaaattgcagcgggagtatgtatgatcgatgctgctctagtttttccagcccacctctcgttgtttgcgacttcgagtttactgcatctatatctctgaaagaaaatgcctacagggcttggccggtgtcagcttcagggctctctgtaggcgttgaatccgaggccgccgtagggtggtggcagcgcgaccatcgagccgtggagcaagtacgagtcaaacacgctgagccgcgtaaccgttttaataaaatgaactctactcgatcgctaggtcacagcagcacgatcacagtgaagaatgcgtgaggggctcgagcgttggcgttgttgctgtgctttgatcaaactgcaccggcgagaaggaagtaatataagcaactcgatcataatccaagcagtaaacgcctgatcaattgtcgacatctggctggcactagtattactagattgtagaatacgccgcgtgatgtgatttgttattttaggtccaaattcatgaagaattgtggagtagttgagtagatgagtacagtgacttgcagttgcagcaaacgattgatgttcacatgacagggttcaggggtcctgtagtcctgttacttgcagtattgccgtatcgtgaatctgaagtcgaacagagatagatcgatggcgtaatgtcccaaggaggagagaaataatatgactgtatcactgtagtgtgtagtcgatgggaatctatagctctcgttattaggctcttaattttaacagaaaccaaagcaactacaagctaattaattccagagacttctacagtgccacatcattcaattggcaccagaattaaaatagtttttcaagaaatcgcatctcagaaaaagatgctaatggtttctttatcgttgtggtcaacactagtgccatccagattacagaattctctcctgatcggatctcatcgcacatggacagccctgcaaacccaacacggaaaccctgaaagatcgcgtccgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 21 - DNA, 3384 bp, synthetic construct
  misc_feature 1..3384: note = Synthetic
  misc_feature 1..3384: note = Root Hybrid 6 PRO
  source 1..3384: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaataccaccacttgtgaactatgaatacatggaagcttgctttgccgataagctagccacaggaaagttcgcaatgggatctaactagcctttggggaaacccattgaagtagagggtttagaaaagcctattgatttggagggtggagaaaccaatggtgaaggttttgtgcaaggtcaagtccccttcgatttaggagttcaaggcctcggagcaagtacagcttcaccttctggctcaacaaataataaaaagaggaagcgggttctgagtgatgaagatgcaattcagataaacaacatgtctcatgcattgcgtgatgtggctggtgctatcaataacacttgccataccgaaacacatcctgatttgtgcaaaacggttatggaccttaccaatttcgacatggatcaacgctcggctgttttggattatttgacggagcataagggtaaaggccttaatttcatgaaaatggaagctgatgtttgtgaagcctcattcaagcgttgttctgtgttgatgcaacttcaaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgctagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgt

SEQ ID NO: 22 - DNA, 3453 bp, synthetic construct
  misc_feature 1..3453: note = Synthetic
  misc_feature 1..3453: note = Root Hybrid 7 PRO
  source 1..3453: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaataccaccacttgtgaactatgaatacatggaagcttgctttgccgataagctagccacaggaaagttcgcaatgggatctaactagcctttggggaaacccattgaagtagagggtttagaaaagcctattgatttggagggtggagaaaccaatggtgaaggttttgtgcaaggtcaagtccccttcgatttaggagttcaaggcctcggagcaagtacagcttcaccttctggctcaacaaataataaaaagaggaagcgggttctgagtgatgaagatgcaattcagataaacaacatgtctcatgcattgcgtgatgtggctggtgctatcaataacacttgccataccgaaacacatcctgatttgtgcaaaacggttatggaccttaccaatttcgacatggatcaacgctcggctgttttggattatttgacggagcataagggtaaaggccttaatttcatgaaaatggaagctgatgtttgtgaagcctcattcaagcgttgttctgtgttgatgcaacttcaaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgctgactatatattacccttctcaagtaaatgggtaagttttaatttgcgccactgattatttgcatggaaaaacataaaccgatattgcaccgacgcgagccccaatttttaaactagcaaatctctagcggcgcctagaaatgaatataatcgggatctatctcttgaaacacgcatagatggcagcacgcggtggagaccaagaagccctggagaaggagccgttgtagctggccggggttcaaaactgaactgcaggagcacgtaccatctcatactagtactaaatctttaatcttctcaagtggagttcctcttgccacccgaagcggaaggaggtcaaccctacgcaccacgaggcacaatagcaaaccgtgtccctctagttaagttaattaccccatgtaaacgtgttcttcctttcagacttgcaatgggtaatgttcacaagtactgtagttgcattactggtaataacgcgatggagtatttagcgatgcaaaatttccaaaacaagcttgtctcgtcagtgacacgatttggcgacactcaacacaacagtttctcacctttacaaatactgcccgttatagatttgttaatactctcttcatctcatattaagtgtcgaaatattacatgtatctagacgctttctagacatagataaattcatatttcgacaactttgagacagttaatatgggacagagggagtacctgagacgagcttagccactctctcttttttcagaacgtaaactcatgcgtgtgactgagtttcttccaagagtttgtaaagaaattaatcgataatgttcagaacttatcctctctctttatatattttcagacacagtatgatccacttgggcatcgagatccacccttccatatagtactatattctactgccacgagcccatatcgagtgtctaccaaactaggagctcgatcgtgatgcacatgaccgaatcacactccacatgcacacaacacatgggatgtgtctaccaaactggctccccttgggtaggtagatatgcaacatttggctagctaaagttctataaataccctcttggcaccccttgcatatctcacacacagcttcttcttctatctatcccacacaaagccttccagctccttaagcctacaactgaccaacc

SEQ ID NO: 23 - DNA, 2529 bp, synthetic construct
  misc_feature 1..2529: note = Synthetic
  misc_feature 1..2529: note = Root Hybrid 8 PRO
  source 1..2529: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgggcactacattactgcggcgcttgctcaaacttcatgcaccaataattgaaacacggtcaattgtatgcattactagtgtaattaagtgcatgtaacgaatgaactaccttaggattttaggagaatcgaagaatatctccatccatttaccaaatcaaacttattaagaagaaaatattatgtacttcctacgtccaaaaaagatgtctcaagtttgtcaaattttggatgtatctagacatgacttaatgtatagatgcattcaaattttgacaaacttaagacatccttttttggacggagggagtacaagatttgttaataagttacattaaaaaactacacagtactgtctactcgtttttttttctaaagaaaatcacggatctattaataatcaccaacatgagccttacttaattaagtacttagagacgacgtcgagcgtttggagagtctttgtacaatgtcaaaggtacctataatccggccggccgggtgatttgaatcgcgcgcctagctagatcgatccatgcatgcgatccacagctagcttgatgcttgcgccagtgtgtgatcgatcatgagctcatgaccccatgacatcgagcgagttagctagagaagccatgcacgcacgttctttcacgtggaagagttataaaaagctagcgtaatacgtttatatatgtcaggagatgggaatctctagctaattaggcttcgagagaaaccaagcaacagctagtaattaacttcagatccttccgcactcgataatcaagtcttgatctgcatagcccagtgaagtgccaataaatctaagatgtgaagagatgtgcaacagtgcaagagaaaatttaatgctgtctagttgcttgatcgatgcagtcaatactagtactgtcaccctgattagaaaaattatcactcatcgaatcacccacttgataattcacagcccgaagtcgtcccgaagcgcgcggtcgcctccttgagatatccaaccccatgcatctcatctaccactataaatactcaggccaccctctctgctaagcaccccaccacctcatctcacacgtactctagctacaaattaagctacgtcggca

SEQ ID NO: 24 - DNA, 2376 bp, synthetic construct
  misc_feature 1..2376: note = Synthetic
  misc_feature 1..2376: note = Root Hybrid 9 PRO
  source 1..2376: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatggtttgacttggaccgactagcactcccccgctaggcacgtagtacgtggtatgtgtggaaacgattggttcgttgtagggagtcagtcagatcagatatgatgcttctcgtgtgttgattttcttcctctgaactgaaaggaagcatgcaagttttacaagctaaactcagcagcggaagctatgcatacgttccactggctgcagaacagggcaggccggcggctagctcctgtgagctgtgttggaggagaataatatcttgtcgtttggctttgctcgggtggatacttttttttcttttgacagctgctcgggtggatactgatacactatgaagaccgaaaaaaagaagcgaagcatataaacagacggatcattgggggccaagccagcaggtagtgcggtggaacattatgtagaatgaatcggttcttgctgtgcagacagctcagctcagcacactcctggcccaaaacaaagtgcatgtggtgtattttcgtatactagctaacaaatgcttaagcccctgtttgaaatattgtattttcggcggacgggcactggcttactgaaatcttgccaggaggcacggacaggtgcttagcagccaagtaatgtacagtaaatccggcaaattgccaggggagtggttcgattcaatcaacgaatgcgattcccgatataaattatccccagtttatttttgattttatacaacgcgtcgccgacgcggtccccaccccgtgcgcgtcgcaaggtccacacgtgtcctctctccgacgcttcctagctcacgccgtccttgattgacctgcctcgccatctataaatggcaaaccaggctaaggctcatttctatactccctcttcctgctctgctctctgctctagtttactccactgacccaaagagagaaaagaaagcactcagaacttgagatcagcatccaggagatcggcc

SEQ ID NO: 25 - DNA, 3280 bp, synthetic construct
  misc_feature 1..3280: note = Synthetic
  misc_feature 1..3280: note = Root Hybrid 10 PRO
  source 1..3280: mol_type = other DNA
caccacttgtgaactatgaatacatggaagcttgctttgccgataagctagccacaggaaagttcgcaatgggatctaactagcctttggggaaacccattgaagtagagggtttagaaaagcctattgatttggagggtggagaaaccaatggtgaaggttttgtgcaaggtcaagtccccttcgatttaggagttcaaggcctcggagcaagtacagcttcaccttctggctcaacaaataataaaaagaggaagcgggttctgagtgatgaagatgcaattcagataaacaacatgtctcatgcattgcgtgatgtggctggtgctatcaataacacttgccataccgaaacacatcctgatttgtgcaaaacggttatggaccttaccaatttcgacatggatcaacgctcggctgttttggattatttgacggagcataagggtaaaggccttaatttcatgaaaatggaagctgatgtttgtgaagcctcattcaagcgttgttctgtgttgatgcaacttcaaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgctgcactacattactgcggcgcttgctcaaacttcatgcaccaataattgaaacacggtcaattgtatgcattactagtgtaattaagtgcatgtaacgaatgaactaccttaggattttaggagaatcgaagaatatctccatccatttaccaaatcaaacttattaagaagaaaatattatgtacttcctacgtccaaaaaagatgtctcaagtttgtcaaattttggatgtatctagacatgacttaatgtatagatgcattcaaattttgacaaacttaagacatccttttttggacggagggagtacaagatttgttaataagttacattaaaaaactacacagtactgtctactcgtttttttttctaaagaaaatcacggatctattaataatcaccaacatgagccttacttaattaagtacttagagacgacgtcgagcgtttggagagtctttgtacaatgtcaaaggtacctataatccggccggccgggtgatttgaatcgcgcgcctagctagatcgatccatgcatgcgatccacagctagcttgatgcttgcgccagtgtgtgatcgatcatgagctcatgaccccatgacatcgagcgagttagctagagaagccatgcacgcacgttctttcacgtggaagagttataaaaagctagcgtaatacgtttatatatgtcaggagatgggaatctctagctaattaggcttcgagagaaaccaagcaacagctagtaattaacttcagatccttccgcactcgataatcaagtcttgatctgcatagcccagtgaagtgccaataaatctaagatgtgaagagatgtgcaacagtgcaagagaaaatttaatgctgtctagttgcttgatcgatgcagtcaatactagtactgtcaccctgattagaaaaattatcactcatcgaatcacccacttgataattcacagcccgaagtcgtcccgaagcgcgcggtcgcctccttgagatatccaacctttgacttggaccgactagcactcccccgctaggcacgtagtacgtggtatgtgtggaaacgattggttcgttgtagggagtcagtcagatcagatatgatgcttctcgtgtgttgattttcttcctctgaactgaaaggaagcatgcaagttttacaagctaaactcagcagcggaagctatgcatacgttccactggctgcagaacagggcaggccggcggctagctcctgtgagctgtgttggaggagaataatatcttgtcgtttggctttgctcgggtggatacttttttttcttttgacagctgctcgggtggatactgatacactatgaagaccgaaaaaaagaagcgaagcatataaacagacggatcattgggggccaagccagcaggtagtgcggtggaacattatgtagaatgaatcggttcttgctgtgcagacagctcagctcagcacactcctggcccaaaacaaagtgcatgtggtgtattttcgtatactagctaacaaatgcttaagcccctgtttgaaatattgtattttcggcggacgggcactggcttactgaaatcttgccaggaggcacggacaggtgcttagcagccaagtaatgtacagtaaatccggcaaattgccaggggagtggttcgattcaatcaacgaatgcgattcccgatataaattatccccagtttatttttgattttatacaacgcgtcgccgacgcggtccccaccccgtgcgcgtcgcaaggtccacacgtgtcctctctccgacgcttcctagctcacgccgtccttgattgacctgcctcgccatctataaatggcaaaccaggctaaggctcatttctatactccctcttcctgctctgctctctgctctagtttactccactgacccaaagagagaaaagaaagcactcagaacttgagatcagcatccaggagatcggcc

SEQ ID NO: 26 - DNA, 3318 bp, synthetic construct
  misc_feature 1..3318: note = Synthetic
  misc_feature 1..3318: note = Root Hybrid 11 PRO
  source 1..3318: mol_type = other DNA
caccacttgtgaactatgaatacatggaagcttgctttgccgataagctagccacaggaaagttcgcaatgggatctaactagcctttggggaaacccattgaagtagagggtttagaaaagcctattgatttggagggtggagaaaccaatggtgaaggttttgtgcaaggtcaagtccccttcgatttaggagttcaaggcctcggagcaagtacagcttcaccttctggctcaacaaataataaaaagaggaagcgggttctgagtgatgaagatgcaattcagataaacaacatgtctcatgcattgcgtgatgtggctggtgctatcaataacacttgccataccgaaacacatcctgatttgtgcaaaacggttatggaccttaccaatttcgacatggatcaacgctcggctgttttggattatttgacggagcataagggtaaaggccttaatttcatgaaaatggaagctgatgtttgtgaagcctcattcaagcgttgttctgtgttgatgcaacttcaaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgctgactatatattacccttctcaagtaaatgggtaagttttaatttgcgccactgattatttgcatggaaaaacataaaccgatattgcaccgacgcgagccccaatttttaaactagcaaatctctagcggcgcctagaaatgaatataatcgggatctatctcttgaaacacgcatagatggcagcacgcggtggagaccaagaagccctggagaaggagccgttgtagctggccggggttcaaaactgaactgcaggagcacgtaccatctcatactagtactaaatctttaatcttctcaagtggagttcctcttgccacccgaagcggaaggaggtcaaccctacgcaccacgaggcacaatagcaaaccgtgtccctctagttaagttaattaccccatgtaaacgtgttcttcctttcagacttgcaatgggtaatgttcacaagtactgtagttgcattactggtaataacgcgatggagtatttagcgatgcaaaatttccaaaacaagcttgtctcgtcagtgacacgatttggcgacactcaacacaacagtttctcacctttacaaatactgcccgttatagatttgttaatactctcttcatctcatattaagtgtcgaaatattacatgtatctagacgctttctagacatagataaattcatatttcgacaactttgagacagttaatatgggacagagggagtacctgagacgagcttagccactctctcttttttcagaacgtaaactcatgcgtgtgactgagtttcttccaagagtttgtaaagaaattaatcgataatgttcagaacttatcctctctctttatatattttcagacacagtatgatccacttgggcatcgagatccacccttccatatagtactatattctactgccacgagcccatatcgagtgtctaccaaactaggagctcgatcgtgatgcacatgaccgaatcacactccacatgcacacaacacatgggatgtgtctaccaaactggctccccttgggtaggtagatatgcaatttgacttggaccgactagcactcccccgctaggcacgtagtacgtggtatgtgtggaaacgattggttcgttgtagggagtcagtcagatcagatatgatgcttctcgtgtgttgattttcttcctctgaactgaaaggaagcatgcaagttttacaagctaaactcagcagcggaagctatgcatacgttccactggctgcagaacagggcaggccggcggctagctcctgtgagctgtgttggaggagaataatatcttgtcgtttggctttgctcgggtggatacttttttttcttttgacagctgctcgggtggatactgatacactatgaagaccgaaaaaaagaagcgaagcatataaacagacggatcattgggggccaagccagcaggtagtgcggtggaacattatgtagaatgaatcggttcttgctgtgcagacagctcagctcagcacactcctggcccaaaacaaagtgcatgtggtgtattttcgtatactagctaacaaatgcttaagcccctgtttgaaatattgtattttcggcggacgggcactggcttactgaaatcttgccaggaggcacggacaggtgcttagcagccaagtaatgtacagtaaatccggcaaattgccaggggagtggttcgattcaatcaacgaatgcgattcccgatataaattatccccagtttatttttgattttatacaacgcgtcgccgacgcggtccccaccccgtgcgcgtcgcaaggtccacacgtgtcctctctccgacgcttcctagctcacgccgtccttgattgacctgcctcgccatctataaatggcaaaccaggctaaggctcatttctatactccctcttcctgctctgctctctgctctagtttactccactgacccaaagagagaaaagaaagcactcagaacttgagatcagcatccaggagatcggcc

SEQ ID NO: 27 - DNA, 3652 bp, synthetic construct
  misc_feature 1..3652: note = Synthetic
  misc_feature 1..3652: note = Root Hybrid 12 PRO
  source 1..3652: mol_type = other DNA
caccacttgtgaactatgaatacatggaagcttgctttgccgataagctagccacaggaaagttcgcaatgggatctaactagcctttggggaaacccattgaagtagagggtttagaaaagcctattgatttggagggtggagaaaccaatggtgaaggttttgtgcaaggtcaagtccccttcgatttaggagttcaaggcctcggagcaagtacagcttcaccttctggctcaacaaataataaaaagaggaagcgggttctgagtgatgaagatgcaattcagataaacaacatgtctcatgcattgcgtgatgtggctggtgctatcaataacacttgccataccgaaacacatcctgatttgtgcaaaacggttatggaccttaccaatttcgacatggatcaacgctcggctgttttggattatttgacggagcataagggtaaaggccttaatttcatgaaaatggaagctgatgtttgtgaagcctcattcaagcgttgttctgtgttgatgcaacttcaaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgcttacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccggactatatattacccttctcaagtaaatgggtaagttttaatttgcgccactgattatttgcatggaaaaacataaaccgatattgcaccgacgcgagccccaatttttaaactagcaaatctctagcggcgcctagaaatgaatataatcgggatctatctcttgaaacacgcatagatggcagcacgcggtggagaccaagaagccctggagaaggagccgttgtagctggccggggttcaaaactgaactgcaggagcacgtaccatctcatactagtactaaatctttaatcttctcaagtggagttcctcttgccacccgaagcggaaggaggtcaaccctacgcaccacgaggcacaatagcaaaccgtgtccctctagttaagttaattaccccatgtaaacgtgttcttcctttcagacttgcaatgggtaatgttcacaagtactgtagttgcattactggtaataacgcgatggagtatttagcgatgcaaaatttccaaaacaagcttgtctcgtcagtgacacgatttggcgacactcaacacaacagtttctcacctttacaaatactgcccgttatagatttgttaatactctcttcatctcatattaagtgtcgaaatattacatgtatctagacgctttctagacatagataaattcatatttcgacaactttgagacagttaatatgggacagagggagtacctgagacgagcttagccactctctcttttttcagaacgtaaactcatgcgtgtgactgagtttcttccaagagtttgtaaagaaattaatcgataatgttcagaacttatcctctctctttatatattttcagacacagtatgatccacttgggcatcgagatccacccttccatatagtactatattctactgccacgagcccatatcgagtgtctaccaaactaggagctcgatcgtgatgcacatgaccgaatcacactccacatgcacacaacacatgggatgtgtctaccaaactggctccccttgggtaggtagatatgcaacatttggctagctaaagttctataaataccctcttggcaccccttgcatatctcacacacagcttcttcttctatctatcccacacaaagccttccagctccttaagcctacaactgaccaacc

SEQ ID NO: 28 - DNA, 2662 bp, synthetic construct
  misc_feature 1..2662: note = Synthetic
  misc_feature 1..2662: note = Root Hybrid 15 PRO
  source 1..2662: mol_type = other DNA
ttagttgcagacggtttttcccctgctaggagatttccgagagaaaaaaaaggcactacaggttaaccaaaaccaccaacctttggagcgtcgaggtcgacgggcatttgcgtagttgaagcttacaaagttgcatatgagatgagtgccggacatgaagcggataacgttttaaactggcaacaatatctagctgtttcaaattcaggcgtgggaagctacgcctacgcgccctggacggcgtgtaaagagccagcatcggcatcattgtcaaacgatcgacaaggccaagaaattccaaatatattattaataaaaaagaaggcacaaattagtttggttttttagtatgtgtggcggaggaaattttgagaacgaacgtatcaaagaaggcacaagacgatatagattgacgcggctagaagttgcagcaagacagtgggtacggtcttatatatcctaataaataaaaaataaaactatagtgtgtcaaatgtcaacaagaggaggaggcagccaaattagcagagggagacaagtagagcacgccttattagcttgcttatttatcgtggtggtgtacttgttaattactggcacgcattatcaacaacgcagttctggatgtgaatctagacaaacatttgtctaggttccgcacgtatagtttttttcctcttttttttggggggggggtgggggggggaacggaagctgtaataaacggtactaggaacgaaagcaaccgccgcgcgcatgtttttgcaatagattacggtgaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgctagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctcgtgggtcgtggcggcctctatataacgccaagacatcgatcagacatccatccatccatccacactcacacagtcgctgtagtagctagcaagcccctaggtgcttgcttgacctactgctctgcccgtgaccagtcgt

SEQ ID NO: 29 - DNA, 2732 bp, synthetic construct
  misc_feature 1..2732: note = Synthetic
  misc_feature 1..2732: note = Root Hybrid 16 PRO-V1
  source 1..2732: mol_type = other DNA
ttagttgcagacggtttttcccctgctaggagatttccgagagaaaaaaaaggcactacaggttaaccaaaaccaccaacctttggagcgtcgaggtcgacgggcatttgcgtagttgaagcttacaaagttgcatatgagatgagtgccggacatgaagcggataacgttttaaactggcaacaatatctagctgtttcaaattcaggcgtgggaagctacgcctacgcgccctggacggcgtgtaaagagccagcatcggcatcattgtcaaacgatcgacaaggccaagaaattccaaatatattattaataaaaaagaaggcacaaattagtttggttttttagtatgtgtggcggaggaaattttgagaacgaacgtatcaaagaaggcacaagacgatatagattgacgcggctagaagttgcagcaagacagtgggtacggtcttatatatcctaataaataaaaaataaaactatagtgtgtcaaatgtcaacaagaggaggaggcagccaaattagcagagggagacaagtagagcacgccttattagcttgcttatttatcgtggtggtgtacttgttaattactggcacgcattatcaacaacgcagttctggatgtgaatctagacaaacatttgtctaggttccgcacgtatagtttttttcctcttttttttggggggggggtgggggggggaacggaagctgtaataaacggtactaggaacgaaagcaaccgccgcgcgcatgtttttgcaatagattacggtgaaatctatatcttttgtaatgcctagtatattatgatgcagacttggttatttggacgtatttcgtatgttagcttttatatattttggtgatgttgacatgctgaattatgtactcaagtaagccttggtaatatatcattttgcggtattttggtgctgtatatttttgctgtaaattatgtttcgaatttatgatgtaatggatgaaatttgaataagtttgtgtggtcattgcaccaagtcagagacgtataatcacatcttactcgcgtgcactctaccaaacaaagtctgagttcaacatgcctaaacagacacagcccaccaaacgacatctcaactcatcatacctcgacagatacagtctgccaaacacatgtgaaaatctcagctcaacttgactagagtcagcttgttagggatgaaatgggaattctggattcaccacggacaccaaacacacccttagatgctcctggatgctccagtgcacgcggtcagccaggagaatagcgtcaaagcaacgtatatctgatatcctggatgctccagtgtaatgtgatattgagtcgattgcaatactttgctcacaggcgatatttcgagaagagaaaaagacacaggaaaagattatatatgtccacttcaacatcatgatctgaacacgtacgtatacaaaaatcatactccgtagcagagactgagaggcatgaaaccagcctcattggcctggacgcgtgaagtttgcgtgcatgcgcatgcgctttttcttgttgctcgactatatattacccttctcaagtaaatgggtaagttttaatttgcgccactgattatttgcatggaaaaacataaaccgatattgcaccgacgcgagccccaatttttaaactagcaaatctctagcggcgcctagaaatgaatataatcgggatctatctcttgaaacacgcatagatggcagcacgcggtggagaccaagaagccctggagaaggagccgttgtagctggccggggttcaaaactgaactgcaggagcacgtaccatctcatactagtactaaatctttaatcttctcaagtggagttcctcttgccacccgaagcggaaggaggtcaaccctacgcaccacgaggcacaatagcaaaccgtgtccctctagttaagttaattaccccatgtaaacgtgttcttcctttcagacttgcaatgggtaatgttcacaagtactgtagttgcattactggtaataacgcgatggagtatttagcgatgcaaaatttccaaaacaagcttgtctcgtcagtgacacgatttggcgacactcaacacaacagtttctcacctttacaaatactgcccgttatagatttgttaatactctcttcatctcatattaagtgtcgaaatattacatgtatctagacgctttctagacatagataaattcatatttcgacaactttgagacagttaatatgggacagagggagtacctgagacgagcttagccactctctcttttttcagaacgtaaactcatgcgtgtgactgagtttcttccaagagtttgtaaagaaattaatcgataatgttcagaacttatcctctctctttatatattttcagacacagtatgatccacttgggcatcgagatccacccttccatatagtactatattctactgccacgagcccatatcgagtgtctaccaaactaggagctcgatcgtgatgcacatgaccgaatcacactccacatgcacacaacacatgggatgtgtctaccaaactggctccccttgggtaggtagatatgcaacatttggctagctaaagttctataaataccctcttggcaccccttgcatatctcacacacagcttcttcttctatctatcccacacaaagccttccagctccttaagcctacaactgaccaacc

SEQ ID NO: 30 - DNA, 2184 bp, synthetic construct
  misc_feature 1..2184: note = Synthetic
  misc_feature 1..2184: note = Root Hybrid 18 PRO
  source 1..2184: mol_type = other DNA
ttagttgcagacggtttttcccctgctaggagatttccgagagaaaaaaaaggcactacaggttaaccaaaaccaccaacctttggagcgtcgaggtcgacgggcatttgcgtagttgaagcttacaaagttgcatatgagatgagtgccggacatgaagcggataacgttttaaactggcaacaatatctagctgtttcaaattcaggcgtgggaagctacgcctacgcgccctggacggcgtgtaaagagccagcatcggcatcattgtcaaacgatcgacaaggccaagaaattccaaatatattattaataaaaaagaaggcacaaattagtttggttttttagtatgtgtggcggaggaaattttgagaacgaacgtatcaaagaaggcacaagacgatatagattgacgcggctagaagttgcagcaagacagtgggtacggtcttatatatcctaataaataaaaaataaaactatagtgtgtcaaatgtcaacaagaggaggaggcagccaaattagcagagggagacaagtagagcacgccttattagcttgcttatttatcgtggtggtgtacttgttaattactggcacgcattatcaacaacgcagttctggatgtgaatctagacaaacatttgtctaggttccgcacgtatagtttttttcctcttttttttggggggggggtgggggggggaacggaagctgtaataaacggtactaggaacgaaagcaaccgccgcgcgcatgtttttgcaatagattacggtgaatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatggtttgacttggaccgactagcactcccccgctaggcacgtagtacgtggtatgtgtggaaacgattggttcgttgtagggagtcagtcagatcagatatgatgcttctcgtgtgttgattttcttcctctgaactgaaaggaagcatgcaagttttacaagctaaactcagcagcggaagctatgcatacgttccactggctgcagaacagggcaggccggcggctagctcctgtgagctgtgttggaggagaataatatcttgtcgtttggctttgctcgggtggatacttttttttcttttgacagctgctcgggtggatactgatacactatgaagaccgaaaaaaagaagcgaagcatataaacagacggatcattgggggccaagccagcaggtagtgcggtggaacattatgtagaatgaatcggttcttgctgtgcagacagctcagctcagcacactcctggcccaaaacaaagtgcatgtggtgtattttcgtatactagctaacaaatgcttaagcccctgtttgaaatattgtattttcggcggacgggcactggcttactgaaatcttgccaggaggcacggacaggtgcttagcagccaagtaatgtacagtaaatccggcaaattgccaggggagtggttcgattcaatcaacgaatgcgattcccgatataaattatccccagtttatttttgattttatacaacgcgtcgccgacgcggtccccaccccgtgcgcgtcgcaaggtccacacgtgtcctctctccgacgcttcctagctcacgccgtccttgattgacctgcctcgccatctataaatggcaaaccaggctaaggctcatttctatactccctcttcctgctctgctctctgctctagtttactccactgacccaaagagagaaaagaaagcactcagaacttgagatcagcatccaggagatcggcc

SEQ ID NO: 31 - DNA, 1151 bp, Zea mays
  misc_feature 1..1151: note = ZM-PCO118362A PRO (TR 1)
  source 1..1151: mol_type = other DNA
aactttcttaatattttccttaattacctcccatacatcttaaatgtctagtggtagcatatatacctccaaagtccaaagctagttgtaagtgacacaaaaaattatgcgtactcattgaacatatctgttacactcactaactagacatgtttagtgcatctacgttagctagttgcagaccccaatcctatattgttcataaatttttatcataaggttccctgctgcaatttagatatccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 32 - DNA, 911 bp, Zea mays
  misc_feature 1..911: note = ZM-PCO118362A PRO (TR 2)
  source 1..911: mol_type = other DNA
atccagcatgctcttcaattttggtgctcacccttacgggtatgccctcactgccttttataattgtataagggaaatattattcaatataatgtcctaaaaattggcaatatcaatctaaaaatcgttatgaataggatgtaaacaaagctactatctgtccatatataacgtcacaggaaggacaaaaaattcagtcagcgatcgagaacggcaaagaaaaaccatattattgttgcttgccgacataaatttaagtataggacaaaaaaaaaagccacatcatattacatactatgggcttaccagacaaaatgaaataaacgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 33 - DNA, 1710 bp, Sorghum bicolor
  misc_feature 1..1710: note = SB-RCC3 PRO [No RCST fix]
  source 1..1710: mol_type = other DNA
ttttaagtatgaccaatttttaagtataaacccctcacgattggttatttttttaagtataaccaatttttaagtataaacccctcaccaatttttaagtataaacctagcgactaataaacacaacttcttgccaaagtgtgagcatcaccattggatctgcgcccctcacgaacagtcttcgccggggtaaaattctccaaattaaagtcatcttgatgtccttgatcacctgtccataaggcccaatcccagctccacgtatacttctgataagattgacatagtcacttgcatgccagtgtggaactctggatgcctaggtcagaggctagtgactggccttcccggcatgctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattcatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccgggtctcaactataaatacccccttggtgacaccgcgatcaaagcatcgcaaacaagcctagctaagagctctctaactacattagatagagtgatctcgagaggtaactggcttgtgatcgagca

SEQ ID NO: 34 - DNA, 1066 bp, Sorghum bicolor
  misc_feature 1..1066: note = SB-TIP2-3 PRO (TR 3)
  source 1..1066: mol_type = other DNA
cctttttggtgtcatcatcttcgtttgtgcaataatactgcaactcaatgaagtcactgcgactcataagagccctgtcatcaccttgcaactggcagttttcaattggtgtaccagcgcaagatccatagattaatcttgtttgcctcacctaccccaaatggaggatgttcactgcattgatgtcacaatccgaccttcttaaccgctctatgtcacaactttaacatgaatgatatatcgtttcctcaaaaacatgaatgatatatcactacagaactcagtatgcagtagattcctgaacgcaagagctgttgcaatgttaatctactaggtgatatcttactcggtaagatactcaggccgaggtccgaggaattacccttttgttgcaggagaaccaagtcatatgaatgaagcagcagctcagtaatgatccaccgactacgctacttctagcatagaattagtaatcttttctaatactgttgtaaatttgtaacaacaaaacaagaaccacgccacggaagaacggtgagaaaaagaaacctttgctaacgtactattcatgaatttgaagattttgtatggatgcatcagtagacgatgcagggtagaaataaaggatcgattcagataatcaatccgcaccgccattgatttgtttagggatgagatgagaattgagaattgggataacatatatctgctccaaggtcttgttctcctcgctgtcttatggtgtatcctaacgtggctcggcctaaacaaccacaattccacagcagtttagcgagttagggcggttggtcggccaatatcagccacaaaacacgaagcaaaatttaaattatcaatcatgtggtgatcattgcacaccgcccatagtattgtattgcacatctgaggcagggctccagcctccaggtccattagcgccttatgatgtgtatttaaggagactgagctgaaggaactctcgcatcagcctgataagctatagccagccatcttctctgaattccaactcagtccaagggctggaagctagaagtaccgtcagaga

SEQ ID NO: 35 - DNA, 2583 bp, synthetic construct
  misc_feature 1..2583: note = Synthetic
  source 1..2583: mol_type = other DNA
cgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccgggtctcaactataaatacccccttggtgacaccgcgatcaaagcatcgcaaacaagcctagctaagagctctctaactacattagatagagtgatctcgagaggtaactggcttgtgatcgagca

SEQ ID NO: 36 - DNA, 2625 bp, synthetic construct
  misc_feature 1..2625: note = Synthetic
  source 1..2625: mol_type = other DNA
ctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatccccgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacttataataattatccgtatagtctagtacgtacgggacatcacgccattgcctgttgtgtataaaaagcgagcatgagctaggttgggagcagagcaaagcgtagtcatcacctgtgtctaggttgggagcaaagcaaagagagagaggaaagctagctagctagcgg

SEQ ID NO: 37 - DNA, 2625 bp, synthetic construct
  misc_feature 1..2625: note = Synthetic
  source 1..2625: mol_type = other DNA
cgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacttataataattatccgtatagtctagtacgtacgggacatcacgccattgcctgttgtgtataaaaagcgagcatgagctaggttgggagcagagcaaagcgtagtcatcacctgtgtctaggttgggagcaaagcaaagagagagaggaaagctagctagctagcgg

SEQ ID NO: 38 - DNA, 3979 bp, synthetic construct
  misc_feature 1..3979: note = Synthetic
  source 1..3979: mol_type = other DNA
agtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggctcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcaaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatggcgtcgctccatctcgcatctataaataccattggccatgcacacccgcactcccacacagcaatacagcacactagcagcagcagcagcagctcgagctagcttagctactacgtgtgtgcaatcagctcgatcgcc

SEQ ID NO: 39 - DNA, 3975 bp, synthetic construct
  misc_feature 1..3975: note = Synthetic
  source 1..3975: mol_type = other DNA
caaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgagtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcctacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggctcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcgagagggcgacgccatgtgtataaaaggggcctaagctgagtggacttgctgcatcagattagtaagcaatctcaagcgcagagagccaaagctttcggtgtagctcgaagagcaaagcgaaggcaaggtcgt

SEQ ID NO: 40 - DNA, 3975 bp, synthetic construct
  misc_feature 1..3975: note = Synthetic
  source 1..3975: mol_type = other DNA
agtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcccaaactcagtttatcaccaaagaccaaacatgtggaaatcagtctctattttgtccaagagcatgtggcccttggagctttgcggcttcttcatgttgctacatctcttcaatatgccgatatatttactaagggttgtcaattgttatcttcatcaacttctgatctaatctcaatgtttgctcctcttccggttgagactactgggggatattagaatatgaatagccaaaaagtcttgtatagtctaaaataaagagtctcaaatagttcacttgagcttaggaaccgaatttgtcgtcagcagtgttttttgctcatagtaaattagccaacaatactttctatcacaccttaacagagtactttctttctgccatggcttatcaaccaacagtattttttgtcaaaagcagtgattatctgtcaatcactagcgccccctctgccggtatatctagcgctcccatcggatctgactagagcagatcttgagcgtgggttggtggctcagggcttgcaggaggcgttggccgtcgccggcgtagagcagtagtcgtaggcggatctgcatcttcaagctctcctccggtcgattcgtgtgagtcttcgacctctgctcaggtcgattcatgccggcgaggggctcagtgctcggctcacgacgcgaaattacgagcggcagcagcaaaccgggctttcaagcccggctctcctcgtgagctgccttagggctcgttcgtttaactattgttcccgatggattcattcctgatgataaaaatagtataaatttacacaatgttcctggctggaatcatttcagacctgcattccatgagaaacgaacggggctttagcgggccacgtgacagtgacgaagggtcgcagtcgctgctggacggactacagacagagaggcgaagcatgcaattgaattttcgctagcggaaagttatcatctaatctccaaccctccttcctacggctggatctgaaaattgacgacctgaacccctgaacggtgccggtagcaattgcaggtctcactcacatgctaaatccagcaaccaaacacgaaggaatatatgtgatctggacagaacatgcaagcgaataatacatagagtcgtaccaaccctacacagttcaacgaattaatcactgggttcacgggcatgctcacgtccaaaatcccagcgacattttataagcgctaagcggaatgatccagacggggccagctcgagcaccacatgtacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggctcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcgagagggcgacgccatgtgtataaaaggggcctaagctgagtggacttgctgcatcagattagtaagcaatctcaagcgcagagagccaaagctttcggtgtagctcgaagagcaaagcgaaggcaaggtcgt

SEQ ID NO: 41 - DNA, 1816 bp, synthetic construct
  misc_feature 1..1816: note = Synthetic
  source 1..1816: mol_type = other DNA
ctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatccccgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 42 - DNA, 2328 bp, synthetic construct
  misc_feature 1..2328: note = Synthetic
  source 1..2328: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccgggtctcaactataaatacccccttggtgacaccgcgatcaaagcatcgcaaacaagcctagctaagagctctctaactacattagatagagtgatctcgagaggtaactggcttgtgatcgagca

SEQ ID NO: 43 - DNA, 2370 bp, synthetic construct
  misc_feature 1..2370: note = Synthetic
  source 1..2370: mol_type = other DNA
ctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacttataataattatccgtatagtctagtacgtacgggacatcacgccattgcctgttgtgtataaaaagcgagcatgagctaggttgggagcagagcaaagcgtagtcatcacctgtgtctaggttgggagcaaagcaaagagagagaggaaagctagctagctagcgg

SEQ ID NO: 44 - DNA, 1560 bp, synthetic construct
  misc_feature 1..1560: note = Synthetic
  source 1..1560: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaataccgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 45 - DNA, 1396 bp, synthetic construct
  misc_feature 1..1396: note = Synthetic
  source 1..1396: mol_type = other DNA
cgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatacttataataattatccgtatagtctagtacgtacgggacatcacgccattgcctgttgtgtataaaaagcgagcatgagctaggttgggagcagagcaaagcgtagtcatcacctgtgtctaggttgggagcaaagcaaagagagagaggaaagctagctagctagcgg

SEQ ID NO: 46 - DNA, 1610 bp, synthetic construct
  misc_feature 1..1610: note = Synthetic
  source 1..1610: mol_type = other DNA
cgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatcccgggtctcaactataaatacccccttggtgacaccgcgatcaaagcatcgcaaacaagcctagctaagagctctctaactacattagatagagtgatctcgagaggtaactggcttgtgatcgagca

SEQ ID NO: 47 - DNA, 1517 bp, synthetic construct
  misc_feature 1..1517: note = Synthetic
  source 1..1517: mol_type = other DNA
tacttaactttactagaggtggacgtagaaagttagtagggcctgctaacttcctagatgaggtcgtaggaagttagtgactctttgaccgcgctcgtttgtcaatagttgaaagctaaatttctagaggaggctgtaggaagttagtggctcgtttgatcgcgctcgtttgtcaacagctgaaagctaactttctagaggagaccgcccctgctaacttcctacgacctttgtggttggggctataaatacccgacctttgtggccttctcaacggagagtaggttatttggaaccaaacctcggaaaacaaatcaccgtgttcatttgtgttgatcttcatcacttgatttgtttcttccctctcctctctctaaagttctcttgctcacattgttttgagtttgcttctaaagttatctgtattgattgagtaactcatagcaaaaagaactatcttttgcactccgaaatattactaacactaaccccgagtataatgcgtgttcaaactttataaatttcagatttcgtctatttatcctctttagacgactttcaaagtttatatttttggtgtaatggctaccacatcaacatgcttgatttcctggcacatgcatttatttttgtcaatacttttgcgtggcccttttcacgtatagaagagtgaagctaacattacattacataaatatttttccctctaattgcggcaatcaagcacgaacgcactagctagtaagctttttgtgaggatttggcatgttgctgtgactgtgagtggattaccgcgacgacagggacgacatgcgtgcccttatctcttggagatttcctcgggcccgaccatcagacgaacaacacatgcatgtgctagcccgtgtcagcaatctagatgacctctcaattttccacacgaggtggggccctcgccacctcatctcttgtcttttcccagttaggtgacatggagcacatgagcctccgagctcaccggggggtgacgccaccccccgtcgacctcgagagagcctagcccacctaagccgagcgtgatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 48 - DNA, 486 bp, Zea mays
  source 1..486: mol_type = other DNA
gagcctagcccacctaagccgagcgtgatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 49 - DNA, 995 bp, Brachypodium distachyon
  source 1..995: mol_type = other DNA
accaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctttgtactcaggtactcacccttaaatctgagcaggcgcttcacttctcaccccccctaaggaaaggctgcaattgcaagcttgtgtcaaagaagagggtagcacctgatcctcttgcctttggagccagaaaca

SEQ ID NO: 50 - DNA, 4259 bp, synthetic construct
  misc_feature 1..4259: note = Synthetic
  source 1..4259: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctctccatgcctccacgccggttataagatagagtttgaggcaacccctcggagtcacaacaac

SEQ ID NO: 51 - DNA, 4289 bp, synthetic construct
  misc_feature 1..4289: note = Synthetic
  source 1..4289: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctaatgccatttggtgtacaactatatattgtaccccattacaccgattgcaggcaccaaatgaaatccaagtcaatcggtgtacattgaccaa

SEQ ID NO: 52 - DNA, 4330 bp, synthetic construct
  misc_feature 1..4330: note = Synthetic
  source 1..4330: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctcaatccccggctctcatctctataagaggagcctttgtattcagttgcaagcatgcaagtcacacactgcaagcttacttctgagcaaaaagagttttgagtgaaataaatttgaagttcccccttacatctt

SEQ ID NO: 53 - DNA, 4087 bp, synthetic construct
  misc_feature 1..4087: note = Synthetic
  source 1..4087: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctgtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctttgtactcaggtactcacccttaaatctgagcaggcgcttcacttctcaccccccctaaggaaaggctgcaattgcaagcttgtgtcaaagaagagggtagcacctgatcctcttgcctttggagccagaaaca

SEQ ID NO: 54 - DNA, 3766 bp, synthetic construct
  misc_feature 1..3766: note = Synthetic
  source 1..3766: mol_type = other DNA
acgtggttagctactaatataaatgtaaggtcattcgatggtttttctattttcaattacctagcattatctcatttctaattgtgataacaaatgcattagaccataattctgtaaatatgtacatttaagcacacagtctatattttaaaattcttctttttgtgtggatatcccaacccaaatccacctctctcttcaatccgtgcatgttcaccgctgccaagtgccaacaacacatcgcatcgtgcatatctttgttggcttgtgcacggtcggcgccaatggaggagacacctgtacggtgcccttggtagaacaacatccttatccctatatgtatggtgcccttcgtagaatgacaccccttatccctacaatagccatgtatgcataccaagaattaaatatactttttcttgaaccacaataatttattatagcggcacttcttgttcaggttgaacacttatttggaacaataaaatgccgagttcctaaccacaggttcacttttttttttccttatcctcctaggaaactaaattttaaaatcataaatttaatttaaatgttaatggaaacaaaaaattatctacaaagacgactcttagccacagccgcctcactgcaccctcaaccacatcctgcaaacagacaccctcgccacatccctccagattcttcactccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaaggagcctcagccgcagccggttccgttgcggttaccgccgatcacatgcccaaggccgcgcctttccgaacgccgagggccgcccgttcccgtgcacagccacacacacagtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagttacttaactttactagaggtggacgtagaaagttagtagggcctgctaacttcctagatgaggtcgtaggaagttagtgactctttgaccgcgctcgtttgtcaatagttgaaagctaaatttctagaggaggctgtaggaagttagtggctcgtttgatcgcgctcgtttgtcaacagctgaaagctaactttctagaggagaccgcccctgctaacttcctacgacctttgtggttggggctataaatacccgacctttgtggccttctcaacggagagtaggttatttggaaccaaacctcggaaaacaaatcaccgtgttcatttgtgttgatcttcatcacttgatttgtttcttccctctcctctctctaaagttctcttgctcacattgttttgagtttgcttctaaagttatctgtattgattgagtaactcatagcaaaaagaactatcttttgcactccgaaatattactaacactaaccccgagtataatgcgtgttcaaactttataaatttcagatttcgtctatttatcctctttagacgactttcaaagtttatatttttggtgtaatggctaccacatcaacatgcttgatttcctggcacatgcatttatttttgtcaatacttttgcgtggcccttttcacgtatagaagagtgaagctaacattacattacataaatatttttccctctaattgcggcaatcaagcacgaacgcactagctagtaagctttttgtgaggatttggcatgttgctgtgactgtgagtggattaccgcgacgacagggacgacatgcgtgcccttatctcttggagatttcctcgggcccgaccatcagacgaacaacacatgcatgtgctagcccgtgtcagcaatctagatgacctctcaattttccacacgaggtggggccctcgccacctcatctcttgtcttttcccagttaggtgacatggagcacatgagcctccgagctcaccggggggtgacgccaccccccgtcgacctcgagagagcctagcccacctaagccgagcgtgatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 55 - DNA, 3760 bp, synthetic construct
  misc_feature 1..3760: note = Synthetic
  source 1..3760: mol_type = other DNA
tacttaactttactagaggtggacgtagaaagttagtagggcctgctaacttcctagatgaggtcgtaggaagttagtgactctttgaccgcgctcgtttgtcaatagttgaaagctaaatttctagaggaggctgtaggaagttagtggctcgtttgatcgcgctcgtttgtcaacagctgaaagctaactttctagaggagaccgcccctgctaacttcctacgacctttgtggttggggctataaatacccgacctttgtggccttctcaacggagagtaggttatttggaaccaaacctcggaaaacaaatcaccgtgttcatttgtgttgatcttcatcacttgatttgtttcttccctctcctctctctaaagttctcttgctcacattgttttgagtttgcttctaaagttatctgtattgattgagtaactcatagcaaaaagaactatcttttgcactccgaaatattactaacactaaccccgagtataatgcgtgttcaaactttataaatttcagatttcgtctatttatcctctttagacgactttcaaagtttatatttttggtgtaatggctaccacatcaacatgcttgatttcctggcacatgcatttatttttgtcaatacttttgcgtggcccttttcacgtatagaagagtgaagctaacattacattacataaatatttttccctctaattgcggcaatcaagcacgaacgcactagctagtaagctttttgtgaggatttggcatgttgctgtgactgtgagtggattaccgcgacgacagggacgacatgcgtgcccttatctcttggagatttcctcgggcccgaccatcagacgaacaacacatgcatgtgctagcccgtgtcagcaatctagatgacctctcaattttccacacgaggtggggccctcgccacctcatctcttgtcttttcccagttaggtgacatggagcacatgagcctccgagctcaccggggggtgacgccaccccccgtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagcacgtggttagctactaatataaatgtaaggtcattcgatggtttttctattttcaattacctagcattatctcatttctaattgtgataacaaatgcattagaccataattctgtaaatatgtacatttaagcacacagtctatattttaaaattcttctttttgtgtggatatcccaacccaaatccacctctctcttcaatccgtgcatgttcaccgctgccaagtgccaacaacacatcgcatcgtgcatatctttgttggcttgtgcacggtcggcgccaatggaggagacacctgtacggtgcccttggtagaacaacatccttatccctatatgtatggtgcccttcgtagaatgacaccccttatccctacaatagccatgtatgcataccaagaattaaatatactttttcttgaaccacaataatttattatagcggcacttcttgttcaggttgaacacttatttggaacaataaaatgccgagttcctaaccacaggttcacttttttttttccttatcctcctaggaaactaaattttaaaatcataaatttaatttaaatgttaatggaaacaaaaaattatctacaaagacgactcttagccacagccgcctcactgcaccctcaaccacatcctgcaaacagacaccctcgccacatccctccagattcttcactccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaaggagcctcagccgcagccggttccgttgcggttaccgccgatcacatgcccaaggccgcgcctttccgaacgccgagggccgcccgttcccgtgcacagccacacacacagtcgagagagcctagcccacctaagccgagcgtgatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 56 - DNA, 2629 bp, synthetic construct
  misc_feature 1..2629: note = Synthetic
  source 1..2629: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagttacttaactttactagaggtggacgtagaaagttagtagggcctgctaacttcctagatgaggtcgtaggaagttagtgactctttgaccgcgctcgtttgtcaatagttgaaagctaaatttctagaggaggctgtaggaagttagtggctcgtttgatcgcgctcgtttgtcaacagctgaaagctaactttctagaggagaccgcccctgctaacttcctacgacctttgtggttggggctataaatacccgacctttgtggccttctcaacggagagtaggttatttggaaccaaacctcggaaaacaaatcaccgtgttcatttgtgttgatcttcatcacttgatttgtttcttccctctcctctctctaaagttctcttgctcacattgttttgagtttgcttctaaagttatctgtattgattgagtaactcatagcaaaaagaactatcttttgcactccgaaatattactaacactaaccccgagtataatgcgtgttcaaactttataaatttcagatttcgtctatttatcctctttagacgactttcaaagtttatatttttggtgtaatggctaccacatcaacatgcttgatttcctggcacatgcatttatttttgtcaatacttttgcgtggcccttttcacgtatagaagagtgaagctaacattacattacataaatatttttccctctaattgcggcaatcaagcacgaacgcactagctagtaagctttttgtgaggatttggcatgttgctgtgactgtgagtggattaccgcgacgacagggacgacatgcgtgcccttatctcttggagatttcctcgggcccgaccatcagacgaacaacacatgcatgtgctagcccgtgtcagcaatctagatgacctctcaattttccacacgaggtggggccctcgccacctcatctcttgtcttttcccagttaggtgacatggagcacatgagcctccgagctcaccggggggtgacgccaccccccgtcgacctcgagagagcctagcccacctaagccgagcgtgatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 57 - DNA, 2385 bp, synthetic construct
  misc_feature 1..2385: note = Synthetic
  source 1..2385: mol_type = other DNA
accaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctgtcgagttacttaactttactagaggtggacgtagaaagttagtagggcctgctaacttcctagatgaggtcgtaggaagttagtgactctttgaccgcgctcgtttgtcaatagttgaaagctaaatttctagaggaggctgtaggaagttagtggctcgtttgatcgcgctcgtttgtcaacagctgaaagctaactttctagaggagaccgcccctgctaacttcctacgacctttgtggttggggctataaatacccgacctttgtggccttctcaacggagagtaggttatttggaaccaaacctcggaaaacaaatcaccgtgttcatttgtgttgatcttcatcacttgatttgtttcttccctctcctctctctaaagttctcttgctcacattgttttgagtttgcttctaaagttatctgtattgattgagtaactcatagcaaaaagaactatcttttgcactccgaaatattactaacactaaccccgagtataatgcgtgttcaaactttataaatttcagatttcgtctatttatcctctttagacgactttcaaagtttatatttttggtgtaatggctaccacatcaacatgcttgatttcctggcacatgcatttatttttgtcaatacttttgcgtggcccttttcacgtatagaagagtgaagctaacattacattacataaatatttttccctctaattgcggcaatcaagcacgaacgcactagctagtaagctttttgtgaggatttggcatgttgctgtgactgtgagtggattaccgcgacgacagggacgacatgcgtgcccttatctcttggagatttcctcgggcccgaccatcagacgaacaacacatgcatgtgctagcccgtgtcagcaatctagatgacctctcaattttccacacgaggtggggccctcgccacctcatctcttgtcttttcccagttaggtgacatggagcacatgagcctccgagctcaccggggggtgacgccaccccccgtcgacctcgagagagcctagcccacctaagccgagcgtgatgaactgatgaacccactaccactagtcaaggcaaaccacaaccacaaatggatcaattgatctagaacaatccgaaggaggggaggccacgtcacactcacaccaaccgaaatatctgccagtatcagatcaaccggccaataggacgccagcgagcccaacacctagcgacgccgcaaaattcaccgcgaggggcaccgggcacggcaaaaacaaaagcccggcgcggtgagaatatctggcgactggcggagacctggtggccagcgcgcggccacatcagccaccccatccgcccacctcacctccggcgagccaatggcaactcgtcttaagattccacgagataaggacccgatcgccggcgacgctatttagccaggtgcgccccccacggtacactccaccagcggcatctatagcaaccggtccaacactttcacgctcagcttcagca

SEQ ID NO: 58 - DNA, 4331 bp, synthetic construct
  misc_feature 1..4331: note = Synthetic
  source 1..4331: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctttgtactcaggtactcacccttaaatctgagcaggcgcttcacttctcaccccccctaaggaaaggctgcaattgcaagcttgtgtcaaagaagagggtagcacctgatcctcttgcctttggagccagaaaca

SEQ ID NO: 59 - DNA, 4051 bp, synthetic construct
  misc_feature 1..4051: note = Synthetic
  source 1..4051: mol_type = other DNA
accaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctgtcgagcaccaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcctgtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataaggtcgagtagcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataagcctggggaatggtttcatatgcatccggctcgcgcaggtttctggcattgagcagtctgcggaatctcgattagtctggaagacatcgctactacccc

SEQ ID NO: 60 - DNA, 1203 bp, Zea mays
  source 1..1203: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataagcctggggaatggtttcatatgcatccggctcgcgcaggtttctggcattgagcagtctgcggaatctcgattagtctggaagacatcgctactacccc

SEQ ID NO: 61 - DNA, 1018 bp, Zea mays
  source 1..1018: mol_type = other DNA
tacttaactttactagaggtggacgtagaaagttagtagggcctgctaacttcctagatgaggtcgtaggaagttagtgactctttgaccgcgctcgtttgtcaatagttgaaagctaaatttctagaggaggctgtaggaagttagtggctcgtttgatcgcgctcgtttgtcaacagctgaaagctaactttctagaggagaccgcccctgctaacttcctacgacctttgtggttggggctataaatacccgacctttgtggccttctcaacggagagtaggttatttggaaccaaacctcggaaaacaaatcaccgtgttcatttgtgttgatcttcatcacttgatttgtttcttccctctcctctctctaaagttctcttgctcacattgttttgagtttgcttctaaagttatctgtattgattgagtaactcatagcaaaaagaactatcttttgcactccgaaatattactaacactaaccccgagtataatgcgtgttcaaactttataaatttcagatttcgtctatttatcctctttagacgactttcaaagtttatatttttggtgtaatggctaccacatcaacatgcttgatttcctggcacatgcatttatttttgtcaatacttttgcgtggcccttttcacgtatagaagagtgaagctaacattacattacataaatatttttccctctaattgcggcaatcaagcacgaacgcactagctagtaagctttttgtgaggatttggcatgttgctgtgactgtgagtggattaccgcgacgacagggacgacatgcgtgcccttatctcttggagatttcctcgggcccgaccatcagacgaacaacacatgcatgtgctagcccgtgtcagcaatctagatgacctctcaattttccacacgaggtggggccctcgccacctcatctcttgtcttttcccagttaggtgacatggagcacatgagcctccgagctcaccggggggtgacgccacccccc

SEQ ID NO: 62 - DNA, 1105 bp, Zea mays
  source 1..1105: mol_type = other DNA
agcccactataaaagggctaaaatagcaaatttttccctgcgtccgtgcacccacgccgagcttgccgctgcgaccgtggccgagcagatcacgcacacgacatgcattgctttgttcgtcttcaaaacaaacccaaaaaaaatggttttgcggttttgcccgaccagcctggagcacggtggtgctcagccagacagccaataggacattcttgattcttgatgttgtattgtatttgttgagtttgtgatctaaattttgtttataggacatattagcaagtaaagcggaggcttgttataggtttgggccggagtatattgctgactctaattagggtttctgttgcgtcataatttttactcctcttataagccgtcgtacaaagacggcgttatgtaaacatttctacatatagtgaaggttttcgtaatttttctctttgcattaaggacctgtttggttagaggtctctcaggcacttgcctgccaagtaaggaatcctagcccatgtattcaaaatctaacgcctggaatctgtgcaactttggccggctccaaactaaataagccttaaaagaatttctatgtaaatctatgtatggtgctctgtggtttgttgtttgatcttccctgttttgtctgctaacggtatccatccgtccattggtgctccggccctttcccaatgtcctgtgctggatcttgatagcttgggcagttgagtttggcgtgtgaagtggacgtggacttggcgagcgtcagtcagcacttcctgaatgaggctatagcttctgcaagctttcgtggatagattttccgagattctcagcttgcatccgatccagcaagctctcagttccgtttagccccccactaatgactaaaagtagactgaaaagttgagacgcgcgcacatgcagctacgccgctacgctatggctggttcacccaccggagcctgcaacgcgctccctgtggagagagaaaagaagctctgccacccgtccacacattcctgtgatagatcgcattcatccagctggctactgaatgcgcgttgtgctgcgtaggacacgtatggcgagctagcctacagataag

SEQ ID NO: 63 - DNA, 1130 bp, Zea mays
  source 1..1130: mol_type = other DNA
acgtggttagctactaatataaatgtaaggtcattcgatggtttttctattttcaattacctagcattatctcatttctaattgtgataacaaatgcattagaccataattctgtaaatatgtacatttaagcacacagtctatattttaaaattcttctttttgtgtggatatcccaacccaaatccacctctctcttcaatccgtgcatgttcaccgctgccaagtgccaacaacacatcgcatcgtgcatatctttgttggcttgtgcacggtcggcgccaatggaggagacacctgtacggtgcccttggtagaacaacatccttatccctatatgtatggtgcccttcgtagaatgacaccccttatccctacaatagccatgtatgcataccaagaattaaatatactttttcttgaaccacaataatttattatagcggcacttcttgttcaggttgaacacttatttggaacaataaaatgccgagttcctaaccacaggttcacttttttttttccttatcctcctaggaaactaaattttaaaatcataaatttaatttaaatgttaatggaaacaaaaaattatctacaaagacgactcttagccacagccgcctcactgcaccctcaaccacatcctgcaaacagacaccctcgccacatccctccagattcttcactccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaaggagcctcagccgcagccggttccgttgcggttaccgccgatcacatgcccaaggccgcgcctttccgaacgccgagggccgcccgttcccgtgcacagccacacacaca

SEQ ID NO: 64 - DNA, 861 bp, Brachypodium distachyon
  source 1..861: mol_type = other DNA
accaacttgctctgtgatgttaatgtttttggtgattagtgacaagggtgcctaatttcttttctgatgttaatattttgaaagattctcactctgaaaaaaaaatgcatgttcatatccatgtttctctaaaaataatccatagccagtgtgtgatactttctactagttcccactaaatgcattgtgaaattaaattctataaaattttgtaatttctaatatttagtaagggtagaaacagagatttttttcttaattttaaatcatcatgtatcaggagtcgcaaaggtccaggaatgatatgcaagattgctacatggttggtatcctcttaatgtcatccttggcagggagttgtggtggatatgccttctgctgccaggcactggggcacaagaagaatggtgtctgccacacaagccaccctgcaccctacaaacaactcacagctggaatggttatcaccaaaccaatgacagaaaaaactgtgtattccacgtaattgatggttactggcaaaattcatggatgtaatacatcagggcatctcaaccgtcgaaagatgctcatgggcactactcctcggcgaaatgcgcccacagaaacccagaattgttcatcagccaagaccatccttaaggtcaagaatgtccagataatatttatggacggtgcagcgcaaacgataaaattccagtattgcagattttacatggtacacagagaagctaaggaaatcatagagacaagctagtggcagagccaggacaaaaacagaaggtggcaagagagttggagcaaccaaatcacagccattcatatccagaaggccagcctccacctcacaactcatatcct

SEQ ID NO: 65 - DNA, 133 bp, Banana Streak Virus
  source 1..133: mol_type = other DNA; note = (AY)
caatccccggctctcatctctataagaggagcctttgtattcagttgcaagcatgcaagtcacacactgcaagcttacttctgagcaaaaagagttttgagtgaaataaatttgaagttcccccttacatctt

SEQ ID NO: 66 - DNA, 92 bp, Hollyhock Leaf Crumple Virus
  source 1..92: mol_type = other DNA
aatgccatttggtgtacaactatatattgtaccccattacaccgattgcaggcaccaaatgaaatccaagtcaatcggtgtacattgaccaa

SEQ ID NO: 67 - DNA, 62 bp, Bean Yellow Dwarf Virus
  source 1..62: mol_type = other DNA
ctccatgcctccacgccggttataagatagagtttgaggcaacccctcggagtcacaacaac

SEQ ID NO: 68 - DNA, 2165 bp, Zea mays
  source 1..2165: mol_type = other DNA
ttcgttaccaactgggttgcataggatttcatgattaagagtgtgtttggtttagctgtgagttttctcctatgaaaaaactgttgtgagaaaaaatagttggaagtcgtttagttcaaactgttgtgagttatccactgtaaacaaattgtatattgtttatatacactctgtttaaatatatctcttaatcagtatatataattaaaaaactaatttcacatttgtgttcctaatattttttacaaataaatcattgtttaattccatttgtaataagtttttattaaaattgcttttatttcatttattataaacatttaattgttttaatcctattttagttttaatttattgtatctatttattaatataacgaacttcgataagaaacaaaagcaaggtcaaggtgttttttcaaagtagttgtggaaaagctgaaccccttttattcaacttttagaagcaggaaaacagaaccaaacagaccctaaaaatgtgtgaatttttagcaggttaattattcgcatctctttggtcatgtttaagaggctggaatagatcaactgcaagaacacatagcagagtggatagggggggggggggggagggtcgtcgtctccctatctgacctctcttctgcattggattgcctttttcggtactctatttaaaacttaaaagtacaaatgaggtgccggattgatggagtgatatataagtttgatgtgtttttcacataagtgacaagtattattgaaagagaacatttgcattgctactgtttgcatatgggaaaattgagaattgtatcatgccttggccgatcagttctttacttagctcgatgtaatgcacaatgttgatagtatgtcgaggatctagcgatgtaatggtgttaggacacgtggttagctactaatataaatgtaaggtcattcgatggtttttctattttcaattacctagcattatctcatttctaattgtgataacaaatgcattagaccataattctgtaaatatgtacatttaagcacacagtctatattttaaaattcttctttttgtgtggatatcccaacccaaatccacctctctcttcaatccgtgcatgttcaccgctgccaagtgccaacaacacatcgcatcgtgcatatctttgttggcttgtgcacggtcggcgccaatggaggagacacctgtacggtgcccttggtagaacaacatccttatccctatatgtatggtgcccttcgtagaatgacaccccttatccctacaatagccatgtatgcataccaagaattaaatatactttttcttgaaccacaataatttattatagcggcacttcttgttcaggttgaacacttatttggaacaataaaatgccgagttcctaaccacaggttcacttttttttttccttatcctcctaggaaactaaattttaaaatcataaatttaatttaaatgttaatggaaacaaaaaattatctacaaagacgactcttagccacagccgcctcactgcaccctcaaccacatcctgcaaacagacaccctcgccacatccctccagattcttcactccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaaggagcctcagccgcagccggttccgttgcggttaccgccgatcacatgcccaaggccgcgcctttccgaacgccgagggccgcccgttcccgtgcacagccacacacacacccgcccgccaacgactccccatccctatttgaacccacccgcgcactgcattgatcaccaatcgcatcgcagcagcacgagcagcacgccgtgccgctccaaccatctcgcttccgtgcttagcttccc

SEQ ID NO: 69 - DNA, 587 bp, Zea mays
  source 1..587: mol_type = other DNA
cgtgtgcatgcatgcatgcatggtacgaacgtctggatagagtctccgagctgagtgtggtccgacgtggaagtgtacgtctcaacacacgacgcatgtgaccgacaagggcaagttgaagtctatgcatggatgggcctgagcgccgcgctgaatgaatctggacgggtggtagggcatctcggtgggcaaaacaaataactccgtgtgctgcatggctgcctttggaatctttgcatgcagctgtgtgctgaactgaaacccttcgctctatctatataaacagatgcccttcgctctcgtctcagcaggcagcatcgtctcaagttttgttctcctctcctagctagccagcacctgcagatctgctcgttgccttggtaattcatcatgtagtacgtagcatcagctagtatttatctcaagtatatatatacgcatatgtgtcgtcgcagtactttcccttatctctctatacacactacacgcatacataccaataccatccgtcttaactcttaatctttgcctgcatacgtacactgcacgtacgtactgcagggctactgattttgtggaacgaagcg

SEQ ID NO: 70 - DNA, 1229 bp, Sorghum bicolor
  source 1..1229: mol_type = other DNA
ctagcatgtagcatgccaaggatctggctgctccaggtttgttatgcctgacatcaccatagggatgagagcaagtataataataggctgtaagctttaaatgctcaggtggagaaaaaaaggagaggagaggagagagaaaagtgggctataagcttatagctgtgttagacataagaatcagaaacttcgtatgagagacaggtgagctatatattaataacaaagagctaactattatatgagtgaaccgagagaaggctgtaaaaaaacttacacaatcaacgatcgacattattattaaccttgctctgtcttgcgagacctctttgacaaagctacatcaatgccggccaagtgccttgggatttgggaatggcttctttcctcccttcctcggttgtcccccaaggcctaggcttgccacgctgtattcagtcgcagccgcctttacttttgccctttgtggaagttttgtaataaatggtctgattctatcttcggatagatgaagccggatgtttcatccattatctaaaaaaaagttggttgctttgctgagctaagaaagtgtaatccagagtgctcgtaacgtattaatgtacataactattatctaatataaatcttcttttgtcgcaaaaaaaggtcggcccatcagaacaaatgatcaatgtaaggcccaaaatttgtgtctcaaatgtcatttacgtttccaagctaaacaaaaacacaggattcatataattttgctggtggcttaggcttcgtccaatagtgcttagtttaatttgtatatacctgcaccatggtattcgtctggccttggatcttgcgcatcaattgcctatggacgatgatcgcagccacgccacattcatttttaatcgccatttgcttgacacccaatgcctctgcaccacttgcgcacgctacgcaccgtctgatacgccaagatcccgagctaaaataacacccaatcatcagatgaaaacaagcgcgagtgcgagccagcccatggcagcgatcttggccatttgcggagccaactgaaagccgtgcacaaaatattcgacaccgtataagggaaaacactagttatacgaggtgggcaataatccagatctcggactcttcctaacccggttcacatgcatagcatatatgatggccggccggggttcacatgaacgccatcccgtgccctagtgcactgatttcttaatccc

SEQ ID NO: 71 - DNA, 973 bp, Zea mays
  source 1..973: mol_type = other DNA
ctactcgccttgtggctcctcctgaaccacctgctcttctcctgtgggggggtgtgagacagcaagggtgagctcacacatgatcatagctcaacaagttgtggggaaccagtggacatgaactcacaaaggtgggagttcatgtgatggttcctctagatgctcaacttgttgcattatattacgcaattgctccgacgcttcatcaattaatcctccttagtgatattaaataacggaataatattagagaaataaacaataatctaagacattagcgcataaagatgtgacaaaatgattgagtctggtcatattaccctccttcatcctttattgcataaaagattgtagtttacaccttcggctttacaaaggagagctcgaaggtaatattacagcttcgaaggcggagtgatttgattctcccttgttcaaaaagcgagatctcttcatatcattgtgcctctatttatagtaaccaagtacaatttcatatgaaattacaaacatgctcatggacatgataattccagtgcacatccaaccctgcttgatacaaaacatgctcataatcatgatgattcaagtgcacatccaccctgctcgatacaacagttggcgacctggtgtgagagtcagaccagacgggctttcacaatcgccatgcatgtcattctctcgtggtccacgtgtttattaatattgccattaattggagggaaataaaatcaacaagaatagcttattgatgagtcatatattatgaatacatcttatcatcttaccaaacaaaaacatatgaccgtcgatgacctgaaactagactattcgggatctgcaatgatctgcttgtaaatattaatttgcacatcacgccattgcatgcacatcggcgtgggcattattaatttggattggacgaaaaatcaaccagagggcgtcacccttttgctagttggccttgtaatac

SEQ ID NO: 72 - DNA, 1397 bp, Sorghum bicolor; identical to SEQ ID NO: 5
  source 1..1397: mol_type = other DNA

SEQ ID NO: 73 - DNA, 1172 bp, Setaria italica
  source 1..1172: mol_type = other DNA
tacgcagttgtcctttggtacattcacaagtttgatcttatcatcaccatcagaagttcagaaagtctcgtagaaaacaaatggaaatgaatactgcttacttagctcaaattcatattccgttgttacaggatacttaaaaaaggtaccaaaggctgttcctaatcatacgctgaagtcgttgccaccaatggcagctgtactgtcatattgtcgtggtttttcaattgctgtacctgatgcaaacgtaatgggtttactaatcttgcacccgccgacttcaaaatgaagagtgctaatttggttcacgtcaccatcaccggttcgaactgtctagaatggcaggcaaagatgattggacaggcatgcagggaaaaagagcaccgatgacgatctatgcgagttcccaccattgcgagcaatgattatcagccacacgacttactcttcagagctaaccactgccatgcagagaaaaagtgaagcatattgtcaggatctacaacgaagtgaaacaatcaggcatgctaaagtgctgaaactttactgatctctcatgttggacaacaaagaatacgggaatacatcagcaacgcaactcttgagctttgcttgctgaatgaccagctagaatttccaagcatttacaggaacatgactttaagtttcagaaaaacaaatacaaggccactaagggcatgttcacttcagcttataagccggctgaaaagctgaaacggctgatttgttgtgagaggaaaacactgtttggtggctgataagccggctgaataagctgaagcgaacaggctgtaaataagcgtggggataacatatcctccagatgacaggcaatctgcaacttgcagcgattcaaatgtacgattaacaaaatatttaagcgctacatgagataatatatcctccaattagggcctttagtattgtcattagctcataagcatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccg

SEQ ID NO: 74 - DNA, 1410 bp, Brachypodium distachyon
  source 1..1410: mol_type = other DNA
agtcgtagaggccaattgatgatgtgcctaatagatacatatggtaaggataataatcatttcttactacattattcatacaaaaaataattaagaatcaaaaattatgagaaacacctcttggtgtggtgtagttgtgggtgcatcactccacccattagggtccaaatcttggtgctcacattatgccggggtctcccttacattcttcctatcaattttttttgtaaatctacagtagatgtctataatgaaaattttcaaatatctaaaatagcaacgaaaatctcatatgttacctgtagaagctcaacacttttgtattgcacacaatgttaataaaataaaactcttgctaaaacttgtaatgactacctaataacaacatattgtgttgtatatgaatttaagcccatctaaatattcggaatattcgcttatcattcaaaagatttagatcaacaaaaagaagtgaagaactttatattttggtaggtaaaatgtataacaaaacaaatctttcagaaaatcacttgatatttccaaacacaatacatctaaattgcaataaaaaagaattttagaaaacaaaaacataaaaatatgggtgttgctgtttgaatttcaatactacaaaaggacatatatgtgacgtcatattagtgtcgggcccagcaggaccgccaatgatgtatagcatcagtgttggtcggtgcaaaacccgccactgatatacagctgcgcgtttcccactttcgacctgatgaacatcagtggcgggcgttgcacccgcccgccactaatttttaagtagaggaccttaaatctaagttgacgtatgagaaccattggattaagatataatggcactctcttctcttctacttgctatcgttggattaatatccgacggtcaagcacatcggctcatgtctaacaaaaaaaaggcaacttcttaatagcaaaaccgtaaaaatatatattttattatacaagtctagcccgcgagctgcttggttcaccctgctagttaagatagtaacttgtagctcttcttgttgcgtataagttgttaaacattgtaaaagcctcctcaagtatcatgtatacctgtgatacctcacgacgatttaaacgcacaattgctgtataatggatatagattggttctaggctccagcgatcgattatccatgtaactacgtacaaacgagtaaacctccaaaatcacaccgctgtcacacatcgtctgcacgcagttgcctgaaaccaatccactgcacctagcccacgggttgaataaaaccgcccgcgccggcctcttcaacgtgcatccacgcagtgtgtcattcccgtcacggactctcgtctcatccggccccttctctcgagcaacacccaccaatctcc